(19) United States
(12) Patent Application Publication
(10) Pub. No.: US 2025/0195223 A1
(43) Pub. Date: Jun. 19, 2025

(54) HEART VALVE REPAIR DEVICES AND DELIVERY DEVICES THEREFOR

(71) Applicant: EDWARDS LIFESCIENCES CORPORATION, Irvine, CA (US)

(72) Inventors: Aviv Galon, Tel Aviv (IL); Noam Nir, Pardes-Hanna (IL); Ido Halabi, Givatayim (IL); Meir Abucasis, Caesarea (IL); Itay Avinathan, Tel-Aviv (IL); Rupesh Gajanan Nawalakhe, Trabuco Canyon, CA (US); Jeffrey Michael Koslosky, Coto De Caza, CA (US)

(21) Appl. No.: 19/066,906

(22) Filed: Feb. 28, 2025

Related U.S. Application Data
(63) Continuation of application No. PCT/US2023/031444, filed on Aug. 29, 2023.
(60) Provisional application No. 63/441,146, filed on Jan. 25, 2023.
(60) Provisional application No. 63/403,671, filed on Sep. 2, 2022.

(51) Int. Cl. A61F 2/24

(52) U.S. Cl. CPC A61F 2/246; A61F 2/2466

(57) ABSTRACT

Devices are configured to be positioned within a native heart valve to impede regurgitation through the native heart valve. The devices can include one or more of an expandable coaptation element and/or an extension or blocking member. The expandable coaptation elements can be adjustable in size, shape, or both to control the area of the native heart valve that is filled by the coaptation element. The expandable coaptation elements, extensions, and/or blocking members can be positioned in the native heart valve, such that regurgitant blood flow is blocked or impeded.

### RELATED APPLICATIONS

The present application is a continuation of Patent Cooperation Treaty Application No. PCT/US2023/031444, filed on Aug. 29, 2023, which claims the benefit of U.S. Provisional Application No. 63/403,671 filed on Sep. 2, 2022, and claims the benefit of U.S. Provisional Application No. 63/441,146 filed on Jan. 25, 2023, which are incorporated herein by reference in their entireties.

### BACKGROUND

The native heart valves (i.e., the aortic, pulmonary, tricuspid, and mitral valves) serve critical functions in assuring the forward flow of an adequate supply of blood through the cardiovascular system. These heart valves may be damaged, and thus rendered less effective, for example, by congenital malformations, inflammatory processes, infectious conditions, disease, etc. Such damage to the valves may result in serious cardiovascular compromise or death. Damaged valves can be surgically repaired or replaced during open heart surgery. However, open heart surgeries are highly invasive, and complications may occur. Transvascular techniques can be used to introduce and deploy/implant devices to treat a heart in a manner that is much less invasive than open heart surgery. As one example, a transvascular technique useable for accessing the native mitral and aortic valves is the trans-septal technique. The trans-septal technique comprises advancing a catheter into the right atrium (e.g., inserting a catheter into the right femoral vein, up the inferior vena cava and into the right atrium). The septum is then punctured, and the catheter passed into the left atrium. A similar transvascular technique can be used to deploy/implant a device within the tricuspid valve that begins similarly to the trans-septal technique but stops short of puncturing the septum and instead turns the delivery catheter toward the tricuspid valve in the right atrium.

A healthy heart has a generally conical shape that tapers to a lower apex. The heart is four-chambered and comprises the left atrium, right atrium, left ventricle, and right ventricle. The left and right sides of the heart are separated by a wall generally referred to as the septum. The native mitral valve of the human heart connects the left atrium to the left ventricle. The mitral valve has a very different anatomy than other native heart valves. The mitral valve includes an annulus portion, which is an annular portion of the native valve tissue surrounding the mitral valve orifice, and a pair of cusps, or leaflets, extending downward from the annulus into the left ventricle. The mitral valve annulus may form a “D”-shaped, oval, or otherwise out-of-round cross-sectional shape having major and minor axes. The anterior leaflet may be larger than the posterior leaflet, forming a generally “C”-shaped boundary between the abutting sides of the leaflets when they are closed together.

When operating properly, the anterior leaflet and the posterior leaflet function together as a one-way valve to allow blood to flow only from the left atrium to the left ventricle. The left atrium receives oxygenated blood from the pulmonary veins. When the muscles of the left atrium contract and the left ventricle dilates (also referred to as “ventricular diastole” or “diastole”), the oxygenated blood that is collected in the left atrium flows into the left ventricle. When the muscles of the left atrium relax and the muscles of the left ventricle contract (also referred to as “ventricular systole” or “systole”), the increased blood pressure in the left ventricle urges the sides of the two leaflets together, thereby closing the one-way mitral valve so that blood cannot flow back to the left atrium and is instead expelled out of the left ventricle through the aortic valve. To prevent the two leaflets from prolapsing under pressure and folding back through the mitral annulus toward the left atrium, a plurality of fibrous cords called chordae tendineae tether the leaflets to papillary muscles in the left ventricle.

Valvular regurgitation involves the valve improperly allowing some blood to flow in the wrong direction through the valve. For example, mitral regurgitation occurs when the native mitral valve fails to close properly and blood flows into the left atrium from the left ventricle during the systolic phase of heart contraction. Mitral regurgitation is one of the most common forms of valvular heart disease. Mitral regurgitation may have many different causes, such as leaflet prolapse, dysfunctional papillary muscles, stretching of the mitral valve annulus resulting from dilation of the left ventricle, more than one of these, etc. Mitral regurgitation at a central portion of the leaflets can be referred to as central jet mitral regurgitation and mitral regurgitation nearer to one commissure (i.e., location where the leaflets meet) of the leaflets can be referred to as eccentric jet mitral regurgitation. Central jet regurgitation occurs when the edges of the leaflets do not meet in the middle and thus the valve does not close, and regurgitation is present. Tricuspid regurgitation may be similar, but on the right side of the heart.

### SUMMARY

This summary is meant to provide some examples and is not intended to be limit the scope of the disclosed subject matter in any way. For example, any feature included in an example of this summary is not required by the claims, unless the claims explicitly recite the feature. Also, the features, components, steps, concepts, etc. described in examples in this summary and elsewhere in this disclosure can be combined in a variety of ways. Various features and steps as described elsewhere in this disclosure can be included in the examples summarized here.

Devices for repairing and/or treating a native valve of a patient are disclosed. The devices can be valve repair devices, implantable devices, valve treatment devices, implants, etc. While the device may be described as an implantable device in some examples herein, similar configurations can be used on other devices, e.g., valve repair devices, treatment devices, etc., that are not necessarily implanted and may be removed after treatment.

In some implementations, there is provided a device (e.g., treatment device, repair device, implantable device, implant, etc.) that is configured to be positioned within a native heart valve to allow the native heart valve to form a more effective seal. In some implementations, the device is part of a system (e.g., a valve repair system, valve treatment system, etc.) including a delivery system having a catheter and a control handle wherein the device is coupled to the delivery system.

In some implementations, a device (e.g., treatment device, repair device, implantable device, implant, etc.) includes an anchor portion. In some implementations, the anchor portion includes one anchor. In some implementations the anchor portion includes two anchors. In some implementations, the anchor portion includes three or more anchors.

In some implementations, each anchor includes a plurality of paddles that are each moveable between an open position and a closed position.

In some implementations, an expandable coaptation element (e.g., spacer, gap filler, plug, etc.) for inhibiting regurgitation between native heart valve leaflets can transition between an unexpanded configuration and an expanded configuration.

In some implementations, an expandable coaptation element (e.g., spacer, gap filler, plug, etc.) for inhibiting regurgitation between native heart valve leaflets includes an expandable mechanism. The expandable mechanism can be the same as or similar to any of the expandable mechanisms described anywhere in this disclosure. The expandable mechanism can comprise one or more of a balloon, expandable container, expandable material, self-expanding material, self-expanding frame, stent, mechanically-expandable frame, pivoting and/or scissoring extensions and/or struts, expanding pully mechanism, Hoberman mechanism, cam, worm screw, rack and pinion, foam, etc.

In some implementations, the expandable coaptation element includes two or more shell components.

In some implementations, the expandable mechanism is configured to move between an expanded configuration and an unexpanded or collapsed configuration.

In some implementations, two or more shell components are attached to the expandable mechanism.

In some implementations, a pair of the two or more shell components nest together when the expandable mechanism is in the collapsed configuration.

In some implementations, the expandable mechanism comprises a plurality of struts.

In some implementations, the expandable mechanism is configured to expand in a single direction. In some implementations, the expandable mechanism is configured to expand in multiple directions. In some implementations the expandable mechanism is configured to expand in two opposed directions.

A device (e.g., a treatment device, a repair device, a valve repair device, an implantable device, an implant, etc.) can include the expandable coaptation element and an anchor portion. A system (e.g., a treatment system, a repair system, a valve repair system, a valve treatment system, etc.) can include one or more catheters and the device.

In some implementations, an expandable coaptation element for inhibiting regurgitation between native heart valve leaflets includes an expandable mechanism. The expandable mechanism can be the same as or similar to any of the expandable mechanisms described anywhere in this disclosure. The expandable mechanism can comprise one or more of a balloon, expandable container, expandable material, self-expanding material, self-expanding frame, stent, mechanically-expandable frame, pivoting and/or scissoring extensions and/or struts, expanding pully mechanism, Hoberman mechanism, cam, worm screw, rack and pinion, foam, etc.

In some implementations, an expandable coaptation element for inhibiting regurgitation between native heart valve leaflets includes an expandable sleeve or an expandable frame. In some implementations, the expandable sleeve or the expandable frame is disposed around an expandable mechanism. The expandable mechanism can be the same as or similar to any of the expandable mechanisms described anywhere in this disclosure. The expandable mechanism can comprise one or more of a balloon, expandable container, expandable material, self-expanding material, self-expanding frame, stent, mechanically-expandable frame, pivoting and/or scissoring extensions and/or struts, expanding pully mechanism, Hoberman mechanism, cam, worm screw, rack and pinion, foam, etc.

In some implementations, the expandable mechanism is configured to move between an expanded configuration and an unexpanded or collapsed configuration.

In some implementations, the sleeve (or frame) includes overlapping end portions. In some implementations, the expandable mechanism comprises a plurality of struts.

In some implementations, the expandable mechanism is configured to expand in a single direction. In some implementations, the expandable mechanism is configured to expand in multiple directions. In some implementations, the expandable mechanism is configured to expand in two opposed directions.

A device (e.g., valve repair device, valve treatment device, implant, etc.) can include the expandable coaptation element and an anchor portion. A system (e.g., valve repair system, valve treatment system, etc.) can include one or more catheters and the device.

In some implementations, an expandable coaptation (e.g., spacer, gap filler, plug, etc.) element for inhibiting regurgitation between native heart valve leaflets includes one or more shape changing components. In some implementations, application of a tensile force to the one or more shape changing components changes the one or more shape changing components from a flat configuration to a curved configuration.

In some implementations, the expandable coaptation element includes a compressible fill material disposed between a pair of the one or more shape changing components. In some implementations, the one or more shape changing components have a kirigami configuration. In some implementations, a pair of the one or more shape changing components are parallel and spaced apart in the flat configuration. In some implementations, the shape changing components curve toward one another in the curved configuration.

A device (e.g., valve repair device, valve treatment device, implant, etc.) can include the expandable coaptation element and an anchor portion. A system (e.g., a valve repair system, valve treatment system, etc.) can include one or more catheters and the device.

In some implementations, an expandable coaptation element for inhibiting regurgitation between native heart valve leaflets includes a lattice of cells.

In some implementations, the lattice of cells includes a first controllable cell and a second controllable cell. In some implementations, the first controllable cell is configured such that a size of the first controllable cell can be increased and decreased. In some implementations, the second controllable cell is configured such that a size of the second controllable cell can be increased and decreased.

In some implementations, the size of the first controllable cell is configured to be controlled independently of the size of the second controllable cell.

A device (e.g., valve repair device, valve treatment device, implant, etc.) can include the expandable coaptation element and an anchor portion. A system (e.g., valve repair system, valve treatment system, etc.) can include one or more catheters and the device.

In some implementations, the system includes a first control member and a second control member. In some implementations, the first control element is configured to move in a direction of a height of the first controllable cell. In some implementations, the second control element is configured to move in a direction of a width of the second controllable cell.

In some implementations, an expandable coaptation element (e.g., spacer, gap filler, plug, etc.) for inhibiting regurgitation between native heart valve leaflets includes a receiver, a shape changing element, and a shaft. In some implementations, the shape changing element has a first end disposed in the receiver and a second end disposed outside the receiver.

In some implementations, the shaft is disposed in the receiver and is connected to the first end of the shape changing element. In some implementations, pushing the shaft in the receiver pushes a portion of the shape changing element out of the receiver to increase a size of the shape changing element.

In some implementations, the shape changing component comprises a plurality of wires. In some implementations, the shape changing element comprises a braided or mesh material. In some implementations, the shape changing element has a teardrop shape in an expanded condition. In some implementations, the shape changing component has a substantially cylindrical configuration in a retracted condition. A device (e.g., valve repair device, valve treatment device, implant, etc.) can include the expandable coaptation element and an anchor portion. A system (e.g., valve repair system, valve treatment system, etc.) can include one or more catheters and the device.

In some implementations, a device (e.g., valve repair device, valve treatment device, implant, etc.) comprises an anchor portion and/or an extension or blocking member. The anchor portion is configured to attach to leaflets of a native heart valve.

In some implementations, the extension or blocking member is attached to the anchor portion. In some implementations, the extension or blocking member is configured to impede regurgitant flow through the native heart valve.

In some implementations, the extension or blocking member is expandable.

In some implementations, a system (e.g., a valve repair system, valve treatment system, etc.) includes a catheter, a device (e.g., valve repair device, valve treatment device, implant, etc.), and an extension or blocking member. The device is coupled to the catheter.

In some implementations, the extension or blocking member is configured to slide over the catheter and attach to the device.

In some implementations, the extension or blocking member is expandable. In some implementations, the device comprises a coaptation element (e.g., spacer, gap filler, plug, etc.) configured to engage leaflets of a native heart valve.

In some implementations, an expandable coaptation element (e.g., spacer, gap filler, plug, etc.) for inhibiting regurgitation between native heart valve leaflets includes an expandable frame member and an expandable mechanism mounted within the expandable frame member. The expandable mechanism can be the same as or similar to any of the expandable mechanisms described anywhere in this disclosure. The expandable mechanism can comprise one or more of a balloon, expandable container, expandable material, self-expanding material, self-expanding frame, stent, mechanically-expandable frame, pivoting and/or scissoring extensions and/or struts, expanding pully mechanism, Hoberman mechanism, cam, worm screw, rack and pinion, foam, etc.

In some implementations, the expandable mechanism can include an expandable member or expansion member connected to the expandable frame and an actuation mechanism mounted inside the expandable/expansion member.

In some implementations, the actuation mechanism is configured to move the expandable/expansion member and the expandable frame member between an expanded configuration and a collapsed configuration.

In some implementations, the actuation mechanism can comprise one or more of an actuation wire, actuation element, pivoting link, joint, cam, rack and pinion, worm screw, lever, pulley, articulating arm, etc.

In some implementations, the expandable/expansion member includes a distal end portion, a proximal end portion opposite the distal end portion, and an intermediate portion between the distal end portion and the proximal end portion.

In some implementations, the intermediate portion expands when the expandable frame member moves from the collapsed configuration to the expanded configuration.

In some implementations, the intermediate portion includes a plurality of longitudinal spaced apart strips configured to bend when the intermediate portion expands. In some implementations, the intermediate portion comprises between 4 and 8 equally spaced apart strips. In some implementations, each of the plurality of strips is attached to the frame member.

In some implementations, the actuation mechanism includes a distal member axially fixed relative to the distal end portion of the expandable/expansion member and a proximal member is axially fixed relative to the proximal end portion of the expandable/expansion member.

In some implementations, the distal member includes a pair of projections received within a pair of openings in the distal end portion of the expandable/expansion member.

In some implementations, the distal member is axially fixed to the distal end portion of the expandable/expansion member by a stop positioned within a passage of the expandable/expansion member.

In some implementations, a portion of the proximal member is axially fixed relative to the distal end portion between a stop positioned within a passage of the expandable/expansion member and an end cap received within the passage at the proximal end portion of the expandable/expansion member.

In some implementations, relative movement of the distal member and the proximal member toward each other causes expansion of the intermediate portion.

In some implementations, the distal member and the proximal member are threadably coupled and relative rotation between the distal member and the proximal member causes relative movement of the distal member and the proximal member toward each other.

In some implementations, the distal end of the proximal member is received within a passage of the distal member and the distal end of the proximal member includes male threads that are threadably coupled to female threads in the passage.

In some implementations, a proximal end of the distal member is received within a passage of the proximal member and the distal member is axially moveable within the passage.

In some implementations, the distal member includes one or more locks configured to lock the axially position of the distal member relative to the proximal member.

In some implementations, each of the one or more locks engage a corresponding slot in the proximal member to lock the axially position of the distal member relative to the proximal member.

In some implementations, the proximal end of the distal member includes a coupling portion configured to be engaged by an actuation element extending through the passage in the proximal member to move the distal member is axially moveable within the passage.

In some implementations, the frame member has a height that remains the same between the collapsed configuration and the expanded configuration.

In some implementations, the frame member comprises a plurality of fixed height posts interconnected by a plurality of struts. In some implementations, the plurality of struts are arranged in a plurality of diamond shape patterns. In some implementations, the expandable/expansion member is connected to one or more fixed height posts of the frame.

In some implementations, a stretchable cover covers at least a portion of the frame member. In some implementations, the cover is connected to the frame member.

In some implementations, the cover includes a plurality of spaced apart weave portions connected by a plurality of stretchable portions.

In some implementations, the frame member includes a plurality of fixed height posts interconnected by a plurality of struts. In some implementations, one or more of the weave portions are connected to a corresponding one or more of the posts.

In some implementations, the frame member has six posts, and the cover has six weave portions, each of which is connected to a corresponding one of the posts.

In some implementations, each of the weave portions is connected to a corresponding stretchable portion by a Leno stitch.

In some implementations, the stretchable cover is treated to decrease the permeability of the cover.

The stretchable cover can be coated with a polymer to decrease the permeability of the cover.

In some implementations, one or more polymer strands made from one or more of TPU, silicone, polyolefin and elastic yarn are woven into the stretchable cover to decrease the permeability of the cover.

In some implementations, the expandable coaptation element is part of a system (e.g., valve repair system, valve treatment system, etc.) that includes a delivery system including a catheter and a control handle and a device (e.g., valve repair device, valve treatment device, implant, etc.) coupled to the delivery system.

In some implementations, the device includes an anchor portion configured to attach to leaflets of a native heart valve. In some implementations, the expandable coaptation element is attached to the anchor portion.

In some implementations, a method of inhibiting regurgitation between native heart valve leaflets includes positioning a device (e.g., valve repair device, valve treatment device, implant, etc.) between the valve leaflets, attaching an anchor portion of the device to the valve leaflets, and expanding a coaptation element attached to the anchor portion.

In some implementations, expanding the coaptation element includes expanding an expandable member or expansion member positioned within a frame member to move the frame member from a collapsed position to an expanded position.

In some implementations, the frame member has a height that remains the same in both the collapsed position and the expanded position.

In some implementations, expanding the expandable/expansion member includes bending a plurality of strips on the expandable/expansion member.

In some implementations, expanding the expandable/expansion member includes axially moving one of a distal member and a proximal member relative to the other of the distal member and a proximal member within a passage of the expandable/expansion member.

In some implementations, axially moving one of a distal member and a proximal member relative to the other of the distal member and a proximal member includes rotating the proximal member relative to the distal member.

In some implementations, axially moving one of a distal member and a proximal member relative to the other of the distal member and a proximal member further includes threading the proximal member into a passage of the distal member.

In some implementations, axially moving one of a distal member and a proximal member relative to the other of the distal member and a proximal member includes axially moving the distal member within a passage of the proximal member.

In some implementations, the method includes locking the position of the distal member within the passage of the proximal member. In some implementations, the method includes inhibiting axial movement of the proximal member while allowing rotation movement.

In some implementations, an expandable coaptation element for inhibiting regurgitation between native heart valve leaflets includes an expandable frame or spacer. In some implementations, the expandable frame or spacer is configured to move between an expanded configuration and a collapsed configuration.

In some implementations, an expandable coaptation element for inhibiting regurgitation between native heart valve leaflets includes an expandable mechanism. In some implementations, the expandable mechanism is configured to move between an expanded configuration and a collapsed configuration. The expandable mechanism can be the same as or similar to any of the expandable mechanisms described anywhere in this disclosure. The expandable mechanism can comprise one or more of a balloon, expandable container, expandable material, self-expanding material, self-expanding frame, stent, mechanically-expandable frame, pivoting and/or scissoring extensions and/or struts, expanding pully mechanism, Hoberman mechanism, cam, worm screw, rack and pinion, foam, etc.

In some implementations, the expandable frame is attached around the expandable mechanism.

In some implementations, the expandable mechanism is configured to expand in only two opposite directions.

In some implementations, the expandable frame has a circular cross-section when the expandable mechanism is in the collapsed configuration and the expandable frame has an oval cross-section when the expandable mechanism is in the expanded configuration.

In some implementations, the expandable mechanism comprises a plurality of struts.

Any of the above method(s) can be performed on a living subject (e.g., human or other animal) or on a simulation (e.g., a cadaver, cadaver heart, imaginary person, anthropomorphic ghost, simulator, such as a computer simulator, e.g., with the body parts, tissue, etc. being simulated). With a simulation, the body parts can optionally be referred to as “simulated” (e.g., simulated heart, simulated tissue, etc.) and can comprise, for example, computerized and/or physical representations.

Any of the above systems, assemblies, devices, apparatuses, components, etc. can be sterilized (e.g., with heat, radiation, ethylene oxide, hydrogen peroxide, etc.) to ensure they are safe for use with patients, and the above methods can comprise (or additional methods comprise or consist of) sterilization of one or more systems, devices, apparatuses, components, etc. herein (e.g., with heat, radiation, ethylene oxide, hydrogen peroxide, etc.).

A further understanding of the nature and advantages of the present invention are set forth in the following description and claims, particularly when considered in conjunction with the accompanying drawings in which like parts bear like reference numerals.

### BRIEF DESCRIPTION OF THE DRAWINGS

To further clarify various aspects of examples in the present disclosure, a more particular description of certain examples and implementations will be made by reference to various aspects of the appended drawings. These drawings depict only example implementations of the present disclosure and are therefore not to be considered limiting of the scope of the disclosure. Moreover, while the figures can be drawn to scale for some examples, the figures are not necessarily drawn to scale for all examples. Examples and other features and advantages of the present disclosure will be described and explained with additional specificity and detail through the use of the accompanying drawings in which:

FIG. 1 illustrates a cutaway view of the human heart in a diastolic phase;

FIG. 2 illustrates a cutaway view of the human heart in a systolic phase;

FIG. 3 illustrates a cutaway view of the human heart in a systolic phase showing valve regurgitation;

FIG. 4 is the cutaway view of FIG. 3 annotated to illustrate a natural shape of mitral valve leaflets in the systolic phase;

FIG. 5 illustrates a healthy mitral valve with the leaflets closed as viewed from an atrial side of the mitral valve;

FIG. 6 illustrates a dysfunctional mitral valve with a visible gap between the leaflets as viewed from an atrial side of the mitral valve;

FIG. 7 illustrates a tricuspid valve viewed from an atrial side of the tricuspid valve;

FIGS. 8-14 show an example device or implant, in various stages of deployment;

FIG. 15 shows an example device that is similar to the device illustrated by FIGS. 8-14, but where the paddles are independently controllable;

FIGS. 16-21 show the example device of FIGS. 8-14 being delivered and deployed within a native valve;

FIG. 22 shows a perspective view of an example device in a closed position;

FIG. 23 shows a perspective view of an example device in a closed position;

FIG. 24 illustrates an example device with paddles in an open position;

FIG. 25A illustrates an example device with paddles in a closed position;

FIG. 25B illustrates a top view of an example device;

FIG. 26 illustrates a perspective view of an example device having paddles of adjustable widths;

FIG. 27 is a cross-section of the example device of FIG. 26 in which the device is bisected;

FIG. 28 is another cross-section of the example device of FIG. 26 in which the device is bisected along a plane perpendicular to the plane illustrated in FIG. 28;

FIG. 29 is a schematic illustration of an example catheter assembly coupled to an example device in which an actuation element is coupled to a paddle actuation control and to a driver head of the device;

FIG. 30 is an illustration of the assembly of FIG. 29 with the example device rotated 90 degrees to show the paddle width adjustment element coupled to an inner end of the connector of the device and coupled to a paddle width control;

FIG. 31 illustrates a perspective view of an example expandable coaptation element of a device;

FIG. 32 illustrates a view of an example device having the expandable coaptation element shown in FIG. 31 implanted within a native valve;

FIG. 33 illustrates a perspective view of an example device with an expandable coaptation element having a shell;

FIGS. 34-35 show example constructions of a component of the shell shown in FIG. 33;

FIG. 36 illustrates a front view of the device of FIG. 33;

FIG. 37 illustrates a top view of the device of FIG. 33;

FIG. 38 illustrates a side view of the device of FIG. 33 in an expanded position;

FIG. 39 illustrates a side view of the device of FIG. 33 in a narrowed position;

FIG. 40 illustrates a perspective view of an example implementation of an expandable sleeve of a coaptation element;

FIG. 41 illustrates a top view of the expandable sleeve of the coaptation element of FIG. 40 in a narrowed position;

FIG. 42 illustrates a perspective view of the expandable sleeve of the coaptation element of FIG. 40 when the expandable coaptation element is in an expanded position;

FIG. 43 illustrates a top view of the expandable sleeve of the coaptation element of FIG. 42 in an expanded position;

FIG. 44 illustrates an expandable coaptation element in a non-tensioned condition;

FIG. 45 illustrates an expandable coaptation element in a tensioned position;

FIG. 46-48 illustrate different types of expansion of expandable coaptation elements from a top view;

FIG. 49 illustrates a top view of an example device having an expandable coaptation element of FIGS. 44 and 45 implanted in a native heart valve with the expandable coaptation element in a non-tensioned condition;

FIG. 50 illustrates a side view of the device and native heart valve of FIG. 49;

FIG. 51 illustrates a top view of an example device having an expandable coaptation element of FIGS. 44 and 45 implanted in a native heart valve with the expandable coaptation element in a tensioned condition;

FIG. 52 illustrates a side view of the device and native heart valve of FIG. 51;

FIG. 53 illustrates a perspective view of an example expandable spacer or coaptation element in an expanded condition;

FIG. 54 illustrates a front view of the expandable spacer or coaptation element of FIG. 53;

FIG. 55 illustrates a perspective cross-section view of the example expandable spacer or coaptation element of FIG. 53 taken along the line 598-598 of FIG. 54;

FIG. 56 illustrates a cross-section view of the example expandable spacer or coaptation element of FIG. 53 taken along the line 598-598 of FIG. 54;

FIG. 57 illustrates a perspective view of an example expandable spacer or coaptation element in an expanded condition;

FIG. 58 illustrates a front view of the expandable spacer or coaptation element of FIG. 57;

FIG. 59 illustrates a perspective cross-section view of the example expandable spacer or coaptation element of FIG. 57 taken along the line 602-602 of FIG. 58;

FIG. 60 illustrates a cross-section view of the example expandable spacer or coaptation element of FIG. 57 taken along the line 602-602 of FIG. 58;

FIG. 61 illustrates a perspective view of an example expandable spacer or coaptation element in an unexpanded condition with a latch tube in an unlatched condition.

FIG. 62 illustrates a front view of the expandable spacer or coaptation element of FIG. 61;

FIG. 63 illustrates a perspective cross-section view of the example expandable spacer or coaptation element of FIG. 61 taken along the line 606-606 of FIG. 62;

FIG. 64 illustrates a cross-section view of the example expandable spacer or coaptation element of FIG. 61 taken along the line 606-606 of FIG. 62;

FIG. 65 illustrates a perspective view of the example expandable spacer or coaptation element of FIG. 61 with the latch tube in a latched condition;

FIG. 66 illustrates a front view of the expandable spacer or coaptation element of FIG. 65;

FIG. 67 illustrates a perspective cross-section view of the example expandable spacer or coaptation element of FIG. 65 taken along the line 610-610 of FIG. 66;

FIG. 68 illustrates a cross-section view of the example expandable spacer or coaptation element of FIG. 65 taken along the line 610-610 of FIG. 66;

FIG. 69 illustrates an enlarged detailed view of the example expandable spacer or coaptation element of FIG. 61 taken in area 607 of FIG. 63;

FIG. 70 illustrates an enlarged detailed view of the example expandable spacer or coaptation element of FIG. 65 taken in area 611 of FIG. 67;

FIG. 71 illustrates an example implementation of a portion of an expandable coaptation element;

FIG. 72 illustrates a cell within a frame of the expandable coaptation element of FIG. 71;

FIG. 73 illustrates the cell within a frame of FIG. 72 in elongated position;

FIG. 74 illustrates the cell of FIG. 71 with an example actuation member;

FIG. 75 illustrates an example implementation of an expandable coaptation element for a device;

FIG. 76 illustrates an example implementation of an expandable coaptation element for a device;

FIG. 77 illustrates an example implementation of an expandable coaptation element for a device;

FIG. 78 illustrates a side view of an example implementation example of a device attached to leaflets of a heart valve;

FIG. 79 illustrates a top view of the device and heart valve of FIG. 78;

FIG. 80 illustrates a side view of an example implementation example of a device in an open configuration and an extension or blocking member for a heart valve;

FIG. 81 illustrates the device and cap of FIG. 80 where the devise is in a closed configuration;

FIG. 82 illustrates a perspective view of an example frame member of an expandable coaptation element;

FIG. 83 illustrates a sectioned perspective view of the frame member of FIG. 82;

FIG. 84 illustrates a top view of the frame member of FIG. 82 with an example expandable/expansion member attached;

FIG. 85 illustrates a side sectional view of the expandable/expansion member and the frame member of FIG. 84;

FIG. 86 illustrates a sectioned perspective view of the expandable/expansion member and the frame member of FIG. 84;

FIG. 87 illustrates a perspective view of the expandable/expansion member of FIG. 84 in an expanded configuration;

FIG. 88 illustrates a perspective view of the expandable/expansion member of FIG. 84 in a collapsed configuration;

FIG. 89 illustrates an exploded perspective view of the expandable/expansion member and an example actuation mechanism for the expandable coaptation element;

FIG. 90 illustrates a sectional view of the expandable/expansion member and the actuation mechanism of FIG. 89 taken along the 90-90 line;

FIG. 91 illustrates a sectional view of the expandable/expansion member and the actuation mechanism of FIG. 89 taken along the 91-91 line;

FIG. 92 illustrates a perspective view of the expandable/expansion member and the actuation mechanism of FIG. 89;

FIG. 93 illustrates a front view of the expandable/expansion member and the actuation mechanism of FIG. 89;

FIG. 94 illustrates a front view of an example expandable/expansion member and an example actuation mechanism for an expandable coaptation element;

FIG. 95 illustrates an exploded view of the actuation mechanism of FIG. 94;

FIG. 96 illustrates a sectional view of the expandable/expansion member and the actuation mechanism of FIG. 94 taken along the 96-96 line;

FIG. 97 illustrates a sectional view of the expandable/expansion member and the actuation mechanism of FIG. 94 taken along the 97-97 line;

FIG. 98 illustrates a perspective view of the sectioned expandable/expansion member and the actuation mechanism of FIG. 97;

FIG. 99 illustrates an example expandable/expansion member and an example actuation mechanism for an expandable coaptation element;

FIG. 100 illustrates an example expandable coaptation element in a collapsed configuration;

FIG. 101 illustrates the expandable coaptation element of FIG. 100 in an expanded configuration;

FIG. 102 illustrates a plan view of a portion of an example cover for an expandable coaptation element with the cover in a first state;

FIG. 103 illustrates a plan view of a portion of a stretchable portion of the cover of FIG. 102 with the cover in a second state;

FIG. 104 illustrates a plan view of a portion of a stretchable portion of the cover of FIG. 102;

FIG. 105 illustrates a plan view of a plain weave of an example cover for an expandable coaptation element;

FIG. 106 illustrates a plan view of a portion of an example cover for an expandable coaptation element, where stretchable portions are in a pre-heated state;

FIG. 107 illustrates a plan view of a portion of an example cover for an expandable coaptation element, where stretchable portions are in a post-heated state;

FIG. 108 illustrates a plan view of an example cover having a stretchable portion that is attached to a paddle frame of a device, where the paddle frame is in an expanded position and the cover is in a stretched position;

FIG. 109 illustrates a plan view of an example cover having a stretchable portion that is attached to a paddle frame of a device, where the paddle frame is in a narrowed position and the cover is in a normal position;

FIG. 110 shows a schematic view an example cover for an expandable coaptation element;

FIG. 111 shows a schematic view of a coating being laminated onto a cover material;

FIG. 112 illustrates a perspective view of the expandable/expansion member in a substantially non-expanded configuration;

FIG. 113 illustrates a perspective view of an example frame of an expandable coaptation element;

FIG. 114 illustrates a perspective view of an example expandable/expansion member and an example actuation mechanism for an expandable coaptation element;

FIG. 115 illustrates a cross-sectional view of the expandable/expansion member and actuation mechanism of FIG. 114;

FIG. 116 illustrates a perspective view of an assembly of the expandable/expansion member and actuation mechanism of FIG. 114 and a rail of a valve repair device;

FIG. 117 illustrates a side view of the assembly of FIG. 116;

FIG. 118 illustrates a view of the assembly of FIG. 116 rotated 90 degrees relative to FIG. 117;

FIG. 119 illustrates a perspective view of a valve repair device with an expandable spacer in an unexpanded configuration;

FIG. 120 illustrates a perspective view of the valve repair device of FIG. 119 with the spacer in an expanded configuration;

FIG. 121 illustrates a plan view from an atrial side of a tricuspid valve of the valve repair device of FIG. 119 installed on leaflets of the tricuspid valve; and

FIG. 122 illustrates a perspective view of a valve repair device having the assembly of FIG. 116.

### DETAILED DESCRIPTION

The following description refers to the accompanying drawings, which illustrate example implementations of the present disclosure. Other implementations having different structures and operation do not depart from the scope of the present disclosure.

Some implementations of the present disclosure are directed to systems, devices, methods, etc. for repairing a defective heart valve. For example, some implementations of devices, valve treatment devices, valve repair devices, implantable devices, implants, and systems (including systems for delivery thereof) are disclosed herein, and any combination of these options can be made unless specifically excluded. In other words, individual components of the disclosed devices and systems can be combined unless mutually exclusive or otherwise physically impossible. Further, the treatment techniques, methods, operations, steps, etc. described or suggested herein can be performed on a living subject (e.g., human, other animal, etc.) or on a non-living simulation, such as a cadaver, cadaver heart, simulator, imaginary person, etc.). When performed on a simulation, the body parts, e.g., heart, tissue, valve, etc., can optionally be referred to as “simulated” (e.g., simulated heart, simulated tissue, simulated valve, etc.) and can comprise, for example, computerized and/or physical representations of body parts, tissue, etc.

As described herein, when one or more components are described as being connected, joined, affixed, coupled, attached, or otherwise interconnected, such interconnection can be direct as between the components or can be indirect such as through the use of one or more intermediary components. Also as described herein, reference to a “member,” “component,” or “portion” shall not be limited to a single structural member, component, or element but can include an assembly of components, members, or elements. Also as described herein, the terms “substantially” and “about” are defined as at least close to (and includes) a given value or state (preferably within 10% of, more preferably within 1% of, and most preferably within 0.1% of). The terms “clasp” and “clasp arm” are often used herein with respect to specific examples, but the terms “gripping member” and/or “gripper arm” can be used in place of and function in the same or similar ways, even if not configured in the same way as a typical clasp.

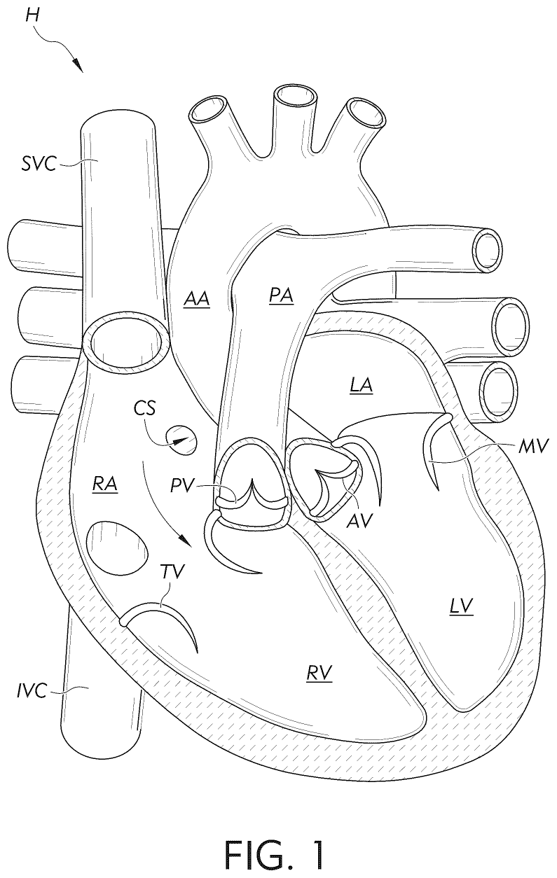

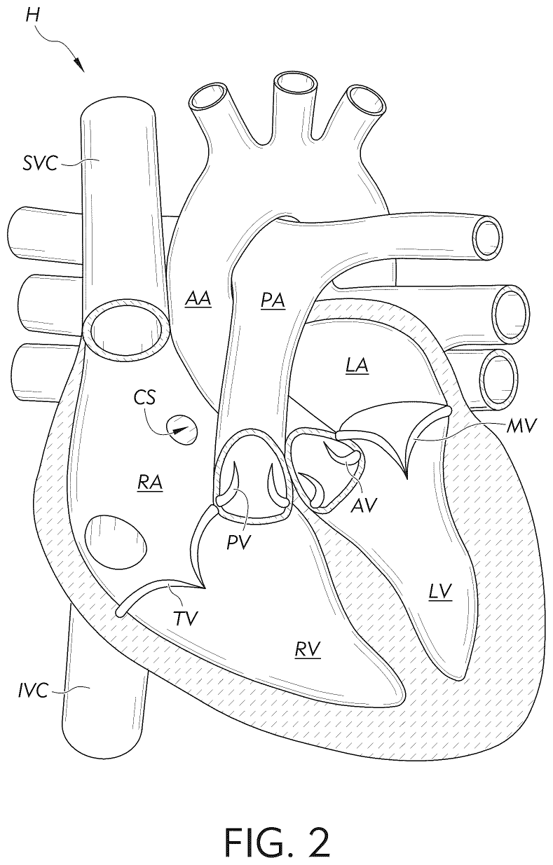

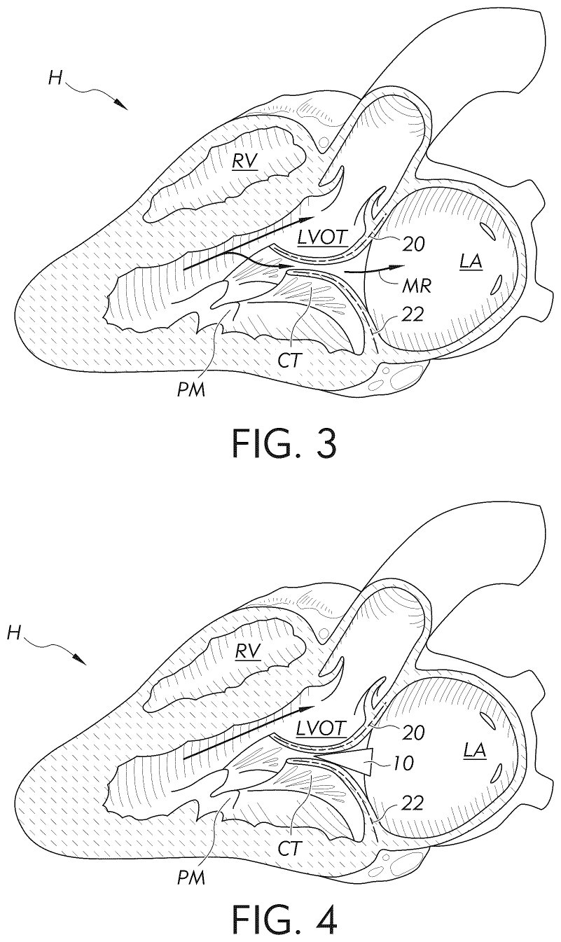

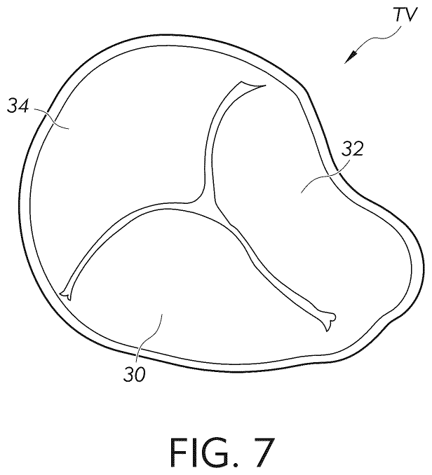

FIGS. 1 and 2 are cutaway views of the human heart H in diastolic and systolic phases, respectively. The right ventricle RV and left ventricle LV are separated from the right atrium RA and left atrium LA, respectively, by the tricuspid valve TV and mitral valve MV; i.e., the atrioventricular valves. Additionally, the aortic valve AV separates the left ventricle LV from the ascending aorta AA, and the pulmonary valve PV separates the right ventricle from the pulmonary artery PA. Each of these valves has flexible leaflets (e.g., leaflets 20, 22 shown in FIGS. 3-6 and leaflets 30, 32, 34 shown in FIG. 7) extending inward across the respective orifices that come together or “coapt” in the flow stream to form the one-way, fluid-occluding surfaces. The native valve repair and/or treatment systems of the present application are frequently described and/or illustrated with respect to the mitral valve MV. Therefore, anatomical structures of the left atrium LA and left ventricle LV will be explained in greater detail. However, the devices described herein can also be used in repairing other native valves, e.g., the devices can be used in repairing the tricuspid valve TV, the aortic valve AV, and the pulmonary valve PV.

The left atrium LA receives oxygenated blood from the lungs. During the diastolic phase, or diastole, seen in FIG. 1, the blood that was previously collected in the left atrium LA (during the systolic phase) moves through the mitral valve MV and into the left ventricle LV by expansion of the left ventricle LV. In the systolic phase, or systole, seen in FIG. 2, the left ventricle LV contracts to force the blood through the aortic valve AV and ascending aorta AA into the body. During systole, the leaflets of the mitral valve MV close to prevent the blood from regurgitating from the left ventricle LV and back into the left atrium LA and blood is collected in the left atrium from the pulmonary vein. In some implementations, the devices described by the present application are used to repair the function of a defective mitral valve MV. That is, the devices are configured to help close the leaflets of the mitral valve to prevent, inhibit or reduce blood from regurgitating from the left ventricle LV and back into the left atrium LA. Many of the devices described in the present application are designed to easily grasp and secure the native leaflets around a coaptation element or spacer that beneficially acts as a filler in the regurgitant orifice to prevent or inhibit back flow or regurgitation during systole, though this is not necessary.

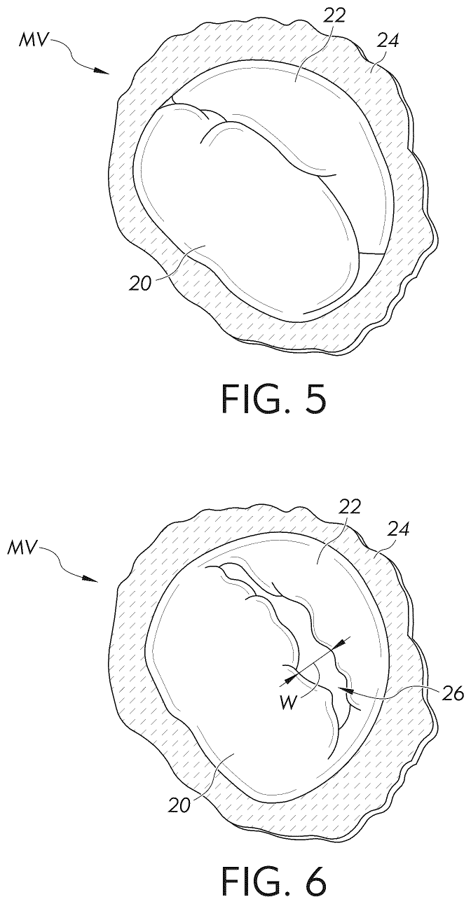

Referring now to FIGS. 1-7, the mitral valve MV includes two leaflets, the anterior leaflet 20 and the posterior leaflet 22. The mitral valve MV also includes an annulus 24 (see FIG. 5), which is a variably dense fibrous ring of tissues that encircles the leaflets 20, 22. Referring to FIGS. 3 and 4, the mitral valve MV is anchored to the wall of the left ventricle LV by chordae tendineae CT. The chordae tendineae CT are cord-like tendons that connect the papillary muscles PM (i.e., the muscles located at the base of the chordae tendineae CT and within the walls of the left ventricle LV) to the leaflets 20, 22 of the mitral valve MV. The papillary muscles PM serve to limit the movements of leaflets 20, 22 of the mitral valve MV and prevent the mitral valve MV from being reverted. The mitral valve MV opens and closes in response to pressure changes in the left atrium LA and the left ventricle LV. The papillary muscles PM do not open or close the mitral valve MV. Rather, the papillary muscles PM support or brace the leaflets 20, 22 against the high pressure needed to circulate blood throughout the body. Together the papillary muscles PM and the chordae tendineae CT are known as the subvalvular apparatus, which functions to keep the mitral valve MV from prolapsing into the left atrium LA when the mitral valve closes. As seen from a Left Ventricular Outflow Tract (LVOT) view shown in FIG. 3, the anatomy of the leaflets 20, 22 is such that the inner sides of the leaflets coapt at the free end portions and the leaflets 20, 22 start receding or spreading apart from each other. The leaflets 20, 22 spread apart in the atrial direction, until each leaflet meets with the mitral annulus.

Various disease processes can impair proper function of one or more of the native valves of the heart H. These disease processes include degenerative processes (e.g., Barlow's Disease, fibroelastic deficiency, etc.), inflammatory processes (e.g., Rheumatic Heart Disease), and infectious processes (e.g., endocarditis, etc.). In addition, damage to the left ventricle LV or the right ventricle RV from prior heart attacks (i.e., myocardial infarction secondary to coronary artery disease) or other heart diseases (e.g., cardiomyopathy, etc.) may distort a native valve's geometry, which may cause the native valve to dysfunction. However, the majority of patients undergoing valve surgery, such as surgery to the mitral valve MV, suffer from a degenerative disease that causes a malfunction in a leaflet (e.g., leaflets 20, 22) of a native valve (e.g., the mitral valve MV), which results in prolapse and regurgitation.

Generally, a native valve may malfunction in different ways: including (1) valve stenosis; and (2) valve regurgitation. Valve stenosis occurs when a native valve does not open completely and thereby causes an obstruction of blood flow. Typically, valve stenosis results from buildup of calcified material on the leaflets of a valve, which causes the leaflets to thicken and impairs the ability of the valve to fully open to permit forward blood flow. Valve regurgitation occurs when the leaflets of the valve do not close completely thereby causing blood to leak back into the prior chamber (e.g., causing blood to leak from the left ventricle to the left atrium).

There are three main mechanisms by which a native valve becomes regurgitant- or incompetent—which include Carpentier's type I, type II, and type III malfunctions. A Carpentier type I malfunction involves the dilation of the annulus such that normally functioning leaflets are distracted from each other and fail to form a tight seal (i.e., the leaflets do not coapt properly). Included in a type I mechanism malfunction are perforations of the leaflets, as are present in endocarditis. A Carpentier's type II malfunction involves prolapse of one or more leaflets of a native valve above a plane of coaptation. A Carpentier's type III malfunction involves restriction of the motion of one or more leaflets of a native valve such that the leaflets are abnormally constrained below the plane of the annulus. Leaflet restriction may be caused by rheumatic disease or dilation of a ventricle.

Referring to FIG. 5, when a healthy mitral valve MV is in a closed position, the anterior leaflet 20 and the posterior leaflet 22 coapt, which prevents blood from leaking from the left ventricle LV to the left atrium LA. Referring to FIGS. 3 and 6, mitral regurgitation MR occurs when the anterior leaflet 20 and/or the posterior leaflet 22 of the mitral valve MV is displaced into the left atrium LA during systole so that the edges of the leaflets 20, 22 are not in contact with each other. This failure to coapt causes a gap 26 between the anterior leaflet 20 and the posterior leaflet 22, which allows blood to flow back into the left atrium LA from the left ventricle LV during systole, as illustrated by the mitral regurgitation MR flow path shown in FIG. 3. Referring to FIG. 6, the gap 26 may have a width W between about 2.5 mm and about 17.5 mm, between about 5 mm and about 15 mm, between about 7.5 mm and about 12.5 mm, or about 10 mm. In some situations, the gap 26 may have a width W greater than 15 mm or even 17.5 mm. As set forth above, there are several different ways that a leaflet (e.g., leaflets 20, 22 of mitral valve MV) may malfunction which may thereby lead to valvular regurgitation.

In any of the above-mentioned situations, a device or implant is desired that is capable of engaging the anterior leaflet 20 and the posterior leaflet 22 to close the gap 26 and prevent or inhibit regurgitation of blood through the mitral valve MV. As can be seen in FIG. 4, an abstract representation of a repair or treatment device 10 (e.g., a valve treatment device, a valve repair device, an implantable device, an implant, etc.) is shown implanted between the leaflets 20, 22 such that regurgitation does not occur during systole (compare FIG. 3 with FIG. 4). In some implementations, the coaptation element (e.g., spacer, coaption element, gap filler, membrane, sheet, plug, wedge, balloon, etc.) of the device 10 has a generally tapered or triangular shape that naturally adapts to the native valve geometry and to its expanding leaflet nature (toward the annulus). In this application, the terms spacer, coaption element, coaptation element, gap filler, plug, etc. are used interchangeably and refer to an element that fills a portion of the space between native valve leaflets and/or that is configured such that the native valve leaflets engage or “coapt” against (e.g., such that the native leaflets coapt against the coaption element, coaptation element, spacer, etc. instead of only against one another).

Although stenosis or regurgitation may affect any valve, stenosis is predominantly found to affect either the aortic valve AV or the pulmonary valve PV, and regurgitation is predominantly found to affect either the mitral valve MV or the tricuspid valve TV. Both valve stenosis and valve regurgitation increase the workload of the heart H and may lead to very serious conditions if left un-treated; such as endocarditis, congestive heart failure, permanent heart damage, cardiac arrest, and ultimately death. Because the left side of the heart (i.e., the left atrium LA, the left ventricle LV, the mitral valve MV, and the aortic valve AV) are primarily responsible for circulating the flow of blood throughout the body. Accordingly, because of the substantially higher pressures on the left side heart dysfunction of the mitral valve MV or the aortic valve AV is particularly problematic and often life threatening.

Malfunctioning native heart valves can either be repaired or replaced. Repair typically involves the preservation and correction of the patient's native valve. Replacement typically involves replacing the patient's native valve with a biological or mechanical substitute. Typically, the aortic valve AV and pulmonary valve PV are more prone to stenosis. Because stenotic damage sustained by the leaflets is irreversible, treatments for a stenotic aortic valve or stenotic pulmonary valve can be removal and replacement of the valve with a surgically implanted heart valve, or displacement of the valve with a transcatheter heart valve. The mitral valve MV and the tricuspid valve TV are more prone to deformation of leaflets and/or surrounding tissue, which, as described above, may prevent the mitral valve MV or tricuspid valve TV from closing properly and allows for regurgitation or back flow of blood from the ventricle into the atrium (e.g., a deformed mitral valve MV may allow for regurgitation or back flow from the left ventricle LV to the left atrium LA as shown in FIG. 3). The regurgitation or back flow of blood from the ventricle to the atrium results in valvular insufficiency. Deformations in the structure or shape of the mitral valve MV or the tricuspid valve TV are often repairable. In addition, regurgitation may occur due to the chordae tendineae CT becoming dysfunctional (e.g., the chordae tendineae CT may stretch or rupture), which allows the anterior leaflet 20 and the posterior leaflet 22 to be reverted such that blood is regurgitated into the left atrium LA. The problems occurring due to dysfunctional chordae tendineae CT can be repaired by repairing the chordae tendineae CT or the structure of the mitral valve MV (e.g., by securing the leaflets 20, 22 at the affected portion of the mitral valve).

The devices and procedures disclosed herein often make reference to repairing the structure of a mitral valve. However, it should be understood that the devices and concepts provided herein can be used to repair any native valve, as well as any component of a native valve. Such devices can be used between the leaflets 20, 22 of the mitral valve MV to prevent or inhibit regurgitation of blood from the left ventricle into the left atrium. With respect to the tricuspid valve TV (FIG. 7), any of the devices and concepts herein can be used between any two of the anterior leaflet 30, septal leaflet 32, and posterior leaflet 34 to prevent or inhibit regurgitation of blood from the right ventricle into the right atrium. In addition, any of the devices and concepts provided herein can be used on all three of the leaflets 30, 32, 34 together to prevent or inhibit regurgitation of blood from the right ventricle to the right atrium. That is, the treatment device, repair devices, implants, etc. provided herein can be centrally located between the three leaflets 30, 32, 34.

An example device (e.g., valve repair device, valve treatment device, implantable device, implant, etc.) can optionally have a coaptation element (e.g., spacer, coaption element, gap filler, membrane, sheet, plug, wedge, balloon, etc.) and at least one anchor (e.g., one, two, three, or more). In some implementations, a device (e.g., a valve repair device, a valve treatment device, an implantable device, an implant, etc.) can have any combination or sub-combination of the features disclosed herein without a coaptation element. When included, the coaptation element (e.g., spacer, coaption element, gap filler, membrane, sheet, plug, wedge, balloon, etc.) is configured to be positioned within the native heart valve orifice to help fill the space between the leaflets and form a more effective seal, thereby reducing or preventing or inhibiting regurgitation described above. The coaptation element can have a structure that is impervious to blood (or that resists blood flow therethrough) and that allows the native leaflets to close around the coaptation element during ventricular systole to block blood from flowing from the left or right ventricle back into the left or right atrium, respectively. The device can be configured to seal against two or three native valve leaflets; that is, the device can be used in the native mitral (bicuspid) and tricuspid valves. The coaptation element is sometimes referred to herein as a spacer because the coaptation element can fill a space between improperly functioning native leaflets (e.g., mitral leaflets 20, 22 or tricuspid leaflets 30, 32, 34) that do not close completely.

The optional coaptation element (e.g., spacer, coaptation element, gap filler, membrane, sheet, plug, wedge, balloon, etc.) can have various shapes. In some implementations, the coaptation element can have an elongated cylindrical shape having a round cross-sectional shape. In some implementations, the coaptation element can have an oval cross-sectional shape, an ovoid cross-sectional shape, a crescent cross-sectional shape, a rectangular cross-sectional shape, or various other non-cylindrical shapes. In some implementations, the coaptation element can have an atrial portion positioned in or adjacent to the atrium, a ventricular or lower portion positioned in or adjacent to the ventricle, and a side surface that extends between the native leaflets. In some implementations configured for use in the tricuspid valve, the atrial or upper portion is positioned in or adjacent to the right atrium, and the ventricular or lower portion is positioned in or adjacent to the right ventricle, and the side surfaces extend between the native tricuspid leaflets.

In some implementations, the anchor can be configured to secure the device to one or both of the native leaflets such that the coaptation element is positioned between the two native leaflets. In some implementations configured for use in the tricuspid valve, the anchor is configured to secure the device to one, two, or three of the tricuspid leaflets such that the coaptation element is positioned between the three native leaflets. In some implementations, the anchor can attach to the coaptation element at a location adjacent the ventricular portion of the coaptation element. In some implementations, the anchor can attach to an actuation element (e.g., an actuation shaft, actuation tube, actuation wire, etc.) to which the coaptation element is also attached. In some implementations, the anchor and the coaptation element can be positioned independently with respect to each other by separately moving each of the anchor and the coaptation element along the longitudinal axis of the actuation element (e.g., actuation shaft, actuation rod, actuation tube, actuation wire, etc.). In some implementations, the anchor and the coaptation element can be positioned simultaneously by moving the anchor and the coaptation element together along the longitudinal axis of the actuation element (e.g., shaft, actuation wire, etc.). The anchor can be configured to be positioned behind a native leaflet when deployed such that the leaflet is grasped by the anchor.

The device can be configured to be deployed and/or implanted via a delivery system or other means for delivery. The delivery system can comprise one or more of a guide/delivery sheath, a delivery catheter, a steerable catheter, an implant catheter, tube, combinations of these, etc. The coaptation element and the anchor can be compressible to a radially compressed state and can be self-expandable to a radially expanded state when compressive pressure is released. The device can be configured for the anchor to be expanded radially away from the still compressed coaptation element initially in order to create a gap between the coaptation element and the anchor. A native leaflet can then be positioned in the gap. The coaptation element can be expanded radially, closing the gap between the coaptation element and the anchor and capturing the leaflet between the coaptation element and the anchor. In some implementations, the anchor and coaptation element are optionally configured to self-expand. The implantation and/or deployment methods for some implementations can be different and are more fully discussed below with respect to each implementation. Additional information regarding these and other delivery methods that can be used with the concepts herein can be found in U.S. Pat. No. 8,449,599 and U.S. Patent Application Publication Nos. 2014/0222136, 2014/0067052, 2016/0331523, PCT patent application publication Nos. WO2020/076898, WO2023/278663, WO2023/004098, WO2023/091520, WO2023/107296, WO2023/086340, WO2023/003755, and WO2022/231889 each of which is incorporated herein by reference in its entirety for all purposes. These method(s) can be performed on a living animal or on a simulation, such as on a cadaver, cadaver heart, simulator (e.g., with the body parts, heart, tissue, etc. being simulated), etc. mutatis mutandis.

The disclosed devices or implants can be configured such that the anchor is connected to a leaflet, taking advantage of the tension from native chordae tendineae to resist high systolic pressure urging the device toward the left atrium. During diastole, the devices can rely on the compressive and retention forces exerted on the leaflet that is grasped by the anchor.

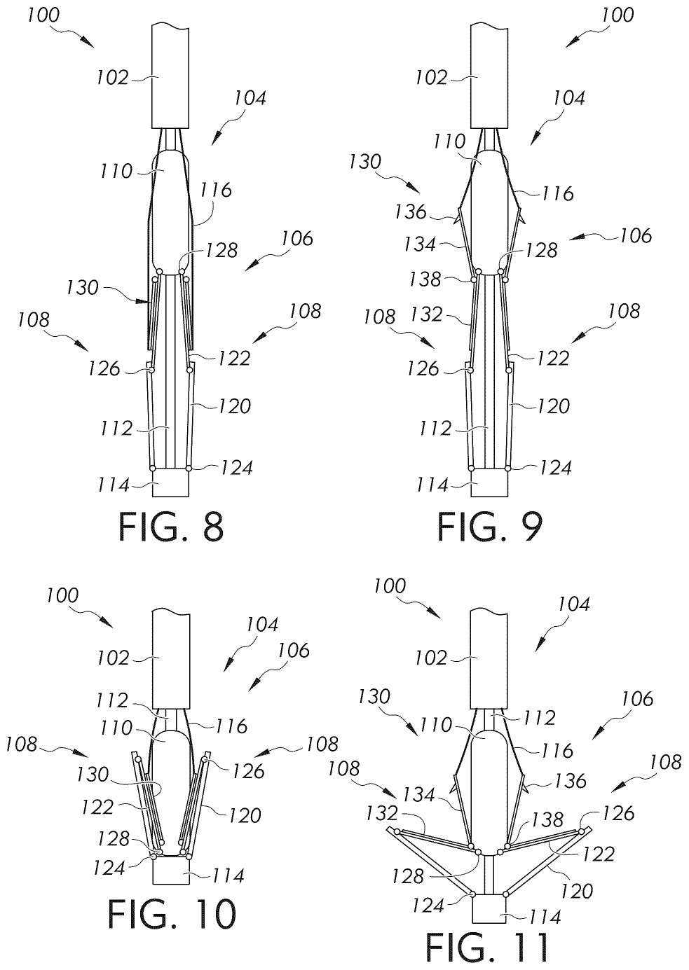

Referring now to FIGS. 8-15, a schematically illustrated device 100 (e.g., a prosthetic device, a valve repair device, valve treatment device, implantable device, implant, etc.) is shown in various stages of deployment. The device 100 and other similar devices and/or implants are described in more detail in PCT patent application publication Nos. WO2018/195215, WO2020/076898, WO2019/139904, WO2023278663, WO2023/004098, WO2023/091520, WO2023/107296, WO2023/086340, WO2023/003755, and WO2022/231889, which are incorporated herein by reference in their entirety for all purposes. The devices herein can include any other features for another device or implant discussed in the present application or the applications cited above, and the devices herein can be positioned to engage valve tissue (e.g., leaflets 20, 22, 30, 32, 34) as part of any suitable treatment and/or repair system (e.g., any valve repair system and/or valve treatment system disclosed in the present application or the applications cited above).

The device 100 is deployed from a delivery system 102. The delivery system 102 can comprise one or more of a catheter, a sheath, a guide catheter/sheath, a delivery catheter/sheath, a steerable catheter, an implant catheter, a tube, a channel, a pathway, combinations of these, etc. The device 100 includes a coaptation portion 104 and an anchor portion 106.

In some implementations, the coaptation portion 104 of the device 100 includes a coaptation element 110 that is adapted to be deployed and/or implanted between leaflets of a native valve (e.g., a native mitral valve, native tricuspid valve, etc.) and is slidably attached to an actuation element 112 (e.g., actuation wire, shaft, tube, hypotube, line, suture, braid, etc.). The anchor portion 106 includes one or more anchors 108 that are actuatable between open and closed conditions and can take a wide variety of forms, such as, for example, paddles, gripping elements, or the like. Actuation of the actuation element 112 opens and closes the anchor portion 106 of the device 100 to grasp the native valve leaflets during deployment and/or implantation. The actuation element 112 (as well as other actuation elements disclosed herein) can take a wide variety of different forms (e.g., as a wire, rod, shaft, tube, screw, suture, line, strip, combination of these, etc.), be made of a variety of different materials, and have a variety of configurations. As one example, the actuation element can be threaded such that rotation of the actuation element moves the anchor portion 106 relative to the coaptation portion 104. Or, the actuation element can be unthreaded, such that pushing or pulling the actuation element 112 moves the anchor portion 106 relative to the coaptation portion 104.

The anchor portion 106 and/or anchors of the device 100 include outer paddles 120 and inner paddles 122 that are, in some implementations, connected between a cap 114 and a coaptation element 110 by portions 124, 126, 128. The portions 124, 126, 128 can be jointed and/or flexible to move between all of the positions described below. The interconnection of the outer paddles 120, the inner paddles 122, the coaptation element 110, and the cap 114 by the portions 124, 126, and 128 can constrain the device to the positions and movements illustrated herein.

In some implementations, the delivery system 102 includes a steerable catheter, implant catheter, and the actuation element 112 (e.g., actuation wire, shaft, tube, hypotube, line, suture, braid, etc.). These can be configured to extend through a guide catheter/sheath (e.g., a transseptal sheath, etc.). In some implementations, the actuation element 112 extends through a delivery catheter and the coaptation element 110 to the distal end (e.g., a cap 114 or other attachment portion at the distal connection of the anchor portion 106). Extending and retracting the actuation element 112 increases and decreases the spacing between the coaptation element 110 and the distal end of the device (e.g., the cap 114 or other attachment portion), respectively. In some implementations, a collar or other attachment element (e.g., clamp, clip, lock, sutures, friction fit, buckle, snap fit, lasso, etc.) removably attaches the coaptation element 110 to the delivery system 102, either directly or indirectly, so that the actuation element 112 slides through the collar or other attachment element and, in some implementations, through a coaptation element 110 during actuation to open and close the paddles 120, 122 of the anchor portion 106 and/or anchors 108.

In some implementations, the anchor portion 106 and/or anchors 108 can include attachment portions or gripping members (e.g., gripping arms, clasp arms, etc.). The illustrated gripping members can comprise clasps 130 that include a base or fixed arm 132, a moveable arm 134, optional friction-enhancing elements, other securing structures 136 (e.g., barbs, protrusions, ridges, grooves, textured surfaces, adhesive, etc.), and a joint portion 138. The fixed arms 132 are attached to the inner paddles 122. In some implementations, the fixed arms 132 are attached to the inner paddles 122 with the joint portion 138 disposed proximate the coaptation element 110. The joint portion 138 provides a spring force between the fixed and moveable arms 132, 134 of the clasp 130. The joint portion 138 can be any suitable joint, such as a flexible joint, a spring joint, a pivot joint, or the like. In some implementations, the joint portion 138 is a flexible piece of material integrally formed with the fixed and moveable arms 132, 134. The fixed arms 132 are attached to the inner paddles 122 and remain stationary or substantially stationary relative to the inner paddles 122 when the moveable arms 134 are opened to open the clasps 130 and expose the optional barbs or other friction-enhancing elements 136.

In some implementations, the clasps 130 are opened by applying tension to actuation lines 116 attached to the moveable arms 134, thereby causing the moveable arms 134 to articulate, flex, or pivot on the joint portions 138. The actuation lines 116 extend through the delivery system 102 (e.g., through a steerable catheter, an implant catheter, etc.). Other actuation mechanisms are also possible.

The actuation line 116 can take a wide variety of forms, such as, for example, a line, a suture, a wire, a rod, a catheter, or the like. The clasps 130 can be spring loaded so that in the closed position the clasps 130 continue to provide a pinching force on the grasped native leaflet. Optional barbs or other friction-enhancing elements 136 of the clasps 130 can grab, pinch, and/or pierce the native leaflets to further secure the native leaflets.

During deployment and/or implantation, the paddles 120, 122 can be opened and closed, for example, to grasp the native leaflets (e.g., native mitral valve leaflets, etc.) between the paddles 120, 122 and/or between the paddles 120, 122 and a coaptation element 110 (e.g., a spacer, plug, membrane, etc.).

The clasps 130 can be used to grasp and/or further secure the native leaflets by engaging the leaflets with optional barbs or other friction-enhancing elements 136 and pinching the leaflets between the moveable and fixed arms 134, 132. The optional barbs or other friction-enhancing elements 136 (e.g., protrusions, ridges, grooves, textured surfaces, adhesive, etc.) of the clasps 130 increase friction with the leaflets or can partially or completely puncture the leaflets.

In some implementations, the actuation lines 116 can be actuated separately (or both separately and simultaneously) so that each clasp 130 can be opened and closed separately. Separate operation allows one leaflet to be grasped at a time, or for the repositioning of a clasp 130 on a leaflet that was insufficiently grasped, without altering a successful grasp on the other leaflet. The clasps 130 can be opened and closed relative to the position of the inner paddle 122 (as long as the inner paddle is in an open or at least partially open position), thereby allowing leaflets to be grasped in a variety of positions as the particular situation requires.

Referring now to FIG. 8, the device 100 is shown in an elongated or fully open condition for deployment from a delivery catheter of the delivery system 102. The device 100 is disposed at the end of the catheter of the delivery system 102 in the fully open position. In the elongated condition the cap 114 is spaced apart from the coaptation element 110 such that the paddles 120, 122 are fully extended. In some implementations, an angle formed between the interior of the outer and inner paddles 120, 122 is approximately 180 degrees. The clasps 130 can be kept in a closed condition during deployment through the delivery system. The actuation lines 116 can extend and attach to the moveable arms 134.

Referring now to FIG. 9, the device 100 is shown in an elongated condition, similar to FIG. 8, but with the clasps 130 in a fully open position, ranging from about 140 degrees to about 200 degrees, from about 170 degrees to about 190 degrees, or about 180 degrees between fixed and moveable arms 132, 134 of the clasps 130.

Referring now to FIG. 10, the device 100 is shown in a shortened or fully closed condition. To move the device 100 from the elongated condition to the shortened condition, the actuation element 112 is retracted to pull the cap 114 towards the coaptation element 110. The connection portion(s) 126 (e.g., joint(s), flexible connection(s), etc.) between the outer paddle 120 and inner paddle 122 are constrained in movement such that compression forces acting on the outer paddle 120 from the cap 114 being retracted towards the coaptation element 110 cause the paddles or gripping elements to move radially outward. During movement from the open position to the closed position, the outer paddles 120 maintain an acute angle with the actuation element 112. The outer paddles 120 can optionally be biased toward a closed position. The inner paddles 122 during the same motion move through a considerably larger angle as they are oriented away from the coaptation element 110 in the open condition and collapse along the sides of the coaptation element 110 in the closed condition.

Referring now to FIGS. 11-13, the device 100 is shown in a partially open, grasp-ready condition. To transition from the fully closed to the partially open condition, the actuation element (e.g., actuation wire, shaft, tube, hypotube, line, suture, braid, etc.) is extended to push the cap 114 away from the coaptation element 110, thereby pulling on the outer paddles 120, which in turn pull on the inner paddles 122, causing the anchors or anchor portion 106 to partially unfold. The actuation lines 116 are also retracted to open the clasps 130 so that the leaflets can be grasped. In some implementations, the pair of inner and outer paddles 122, 120 are moved in unison, rather than independently, by a single actuation element 112. Also, the positions of the clasps 130 are dependent on the positions of the paddles 122, 120. For example, referring to FIG. 10 closing the paddles 122, 120 also closes the clasps. In some implementations, the paddles 120, 122 can be independently controllable. In the example illustrated by FIG. 15, the device 100 can have two actuation elements 111, 113 and two independent caps 115, 117 (or other attachment portions), such that one independent actuation element (e.g., actuation wire, shaft, tube, hypotube, line, suture, braid, etc.) and cap (or other attachment portion) are used to control one paddle, and the other independent actuation element and cap (or other attachment portion) are used to control the other paddle.

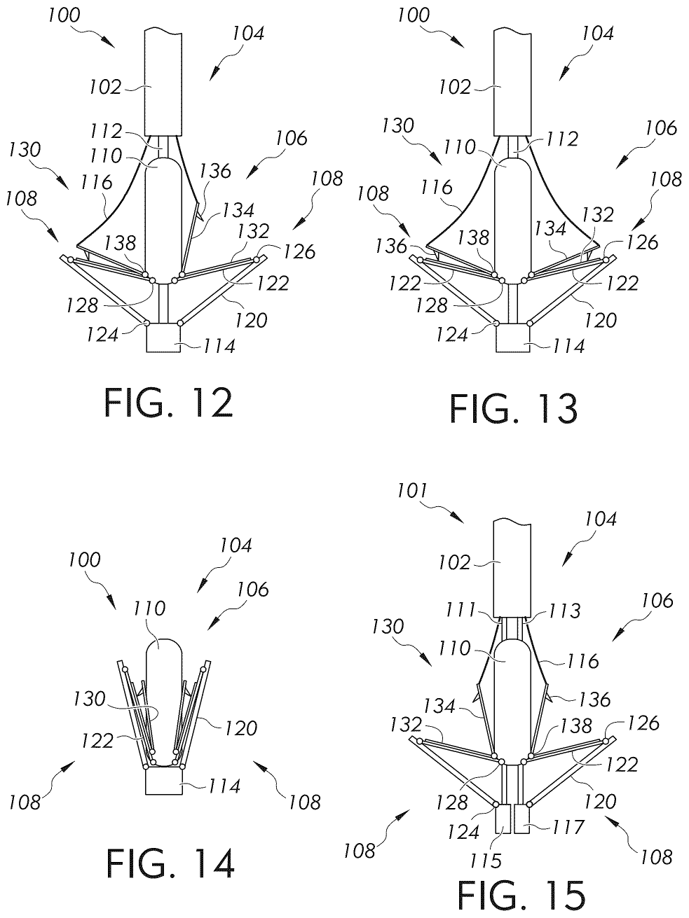

Referring now to FIG. 12, one of the actuation lines 116 is extended to allow one of the clasps 130 to close. Referring now to FIG. 13, the other actuation line 116 is extended to allow the other clasp 130 to close. Either or both of the actuation lines 116 can be repeatedly actuated to repeatedly open and close the clasps 130.

Referring now to FIG. 14, the device 100 is shown in a fully closed and deployed condition. The delivery system 102 and actuation element 112 are retracted and the paddles 120, 122 and clasps 130 remain in a fully closed position. Once deployed, the device 100 can be maintained in the fully closed position with a mechanical latch or can be biased to remain closed through the use of spring materials, such as steel, other metals, plastics, composites, etc. or shape-memory alloys such as Nitinol. For example, the connection portions 124, 126, 128, the joint portions 138, and/or the inner and outer paddles 122, and/or an additional biasing component (not shown) can be formed of metals such as steel or shape-memory alloy, such as Nitinol—produced in a wire, sheet, tubing, or laser sintered powder—and are biased to hold the outer paddles 120 closed around the coaptation element 110 and the clasps 130 pinched around native leaflets. Similarly, the fixed and moveable arms 132, 134 of the clasps 130 are biased to pinch the leaflets. In some implementations, the attachment or connection portions 124, 126, 128, joint portions 138, and/or the inner and outer paddles 122, and/or an additional biasing component (not shown) can be formed of any other suitably elastic material, such as a metal or polymer material, to maintain the device 100 in the closed condition after deployment and/or implantation.

FIG. 15 illustrates an example where the paddles 120, 122 are independently controllable. The device 101 illustrated by FIG. 15 is similar to the device illustrated by FIG. 11, except the device 100 of FIG. 15 includes an actuation element that is configured as two independent actuation elements 111, 113 that are coupled to two independent caps 115, 117. To transition a first inner paddle 122 and a first outer paddle 120 from the fully closed to the partially open condition, the actuation element 111 is extended to push the cap 115 away from the coaptation element 110, thereby pulling on the outer paddle 120, which in turn pulls on the inner paddle 122, causing the first anchor 108 to partially unfold. To transition a second inner paddle 122 and a second outer paddle 120 from the fully closed to the partially open condition, the actuation element 113 is extended to push the cap 115 away from the spacer or coaptation element 110, thereby pulling on the outer paddle 120, which in turn pulls on the inner paddle 122, causing the second anchor 108 to partially unfold. The independent paddle control illustrated by FIG. 15 can be implemented on any of the devices disclosed by the present application. For comparison, in the example illustrated by FIG. 11, the pair of inner and outer paddles 122, 120 are moved in unison, rather than independently, by a single actuation element 112.

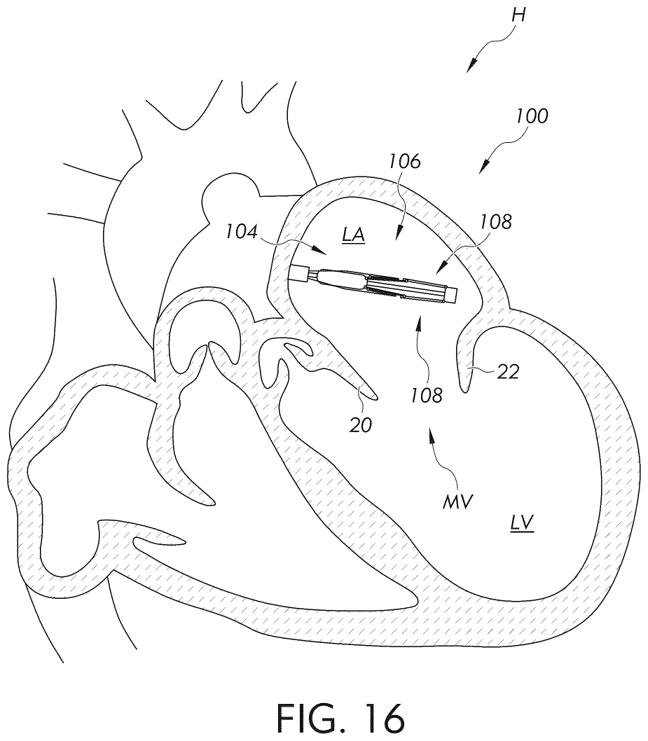

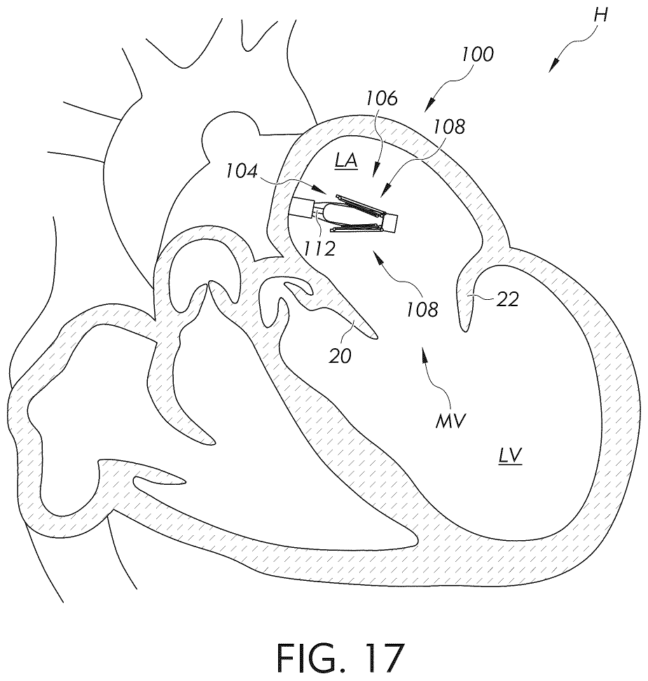

Referring now to FIGS. 16-21, the device 100 of FIGS. 8-14 is shown being delivered and deployed within the native mitral valve MV of the heart H. Referring to FIG. 16, a delivery sheath/catheter is inserted into the left atrium LA through the septum and the implant/device 100 is deployed from the delivery catheter/sheath in the fully open condition as illustrated in FIG. 16. The actuation element 112 is then retracted to move the implant/device into the fully closed condition shown in FIG. 17.

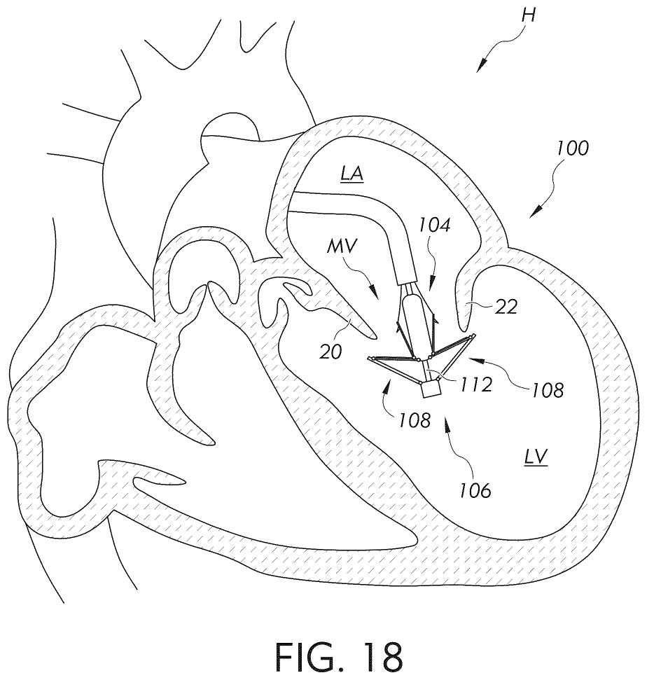

As can be seen in FIG. 18, the implant/device is moved into position within the mitral valve MV into the ventricle LV and partially opened so that the leaflets 20, 22 can be grasped. For example, a steerable catheter can be advanced and steered or flexed to position the steerable catheter as illustrated by FIG. 18. The device or implant catheter connected to the implant/device can be advanced from inside the steerable catheter to position the implant as illustrated by FIG. 18.

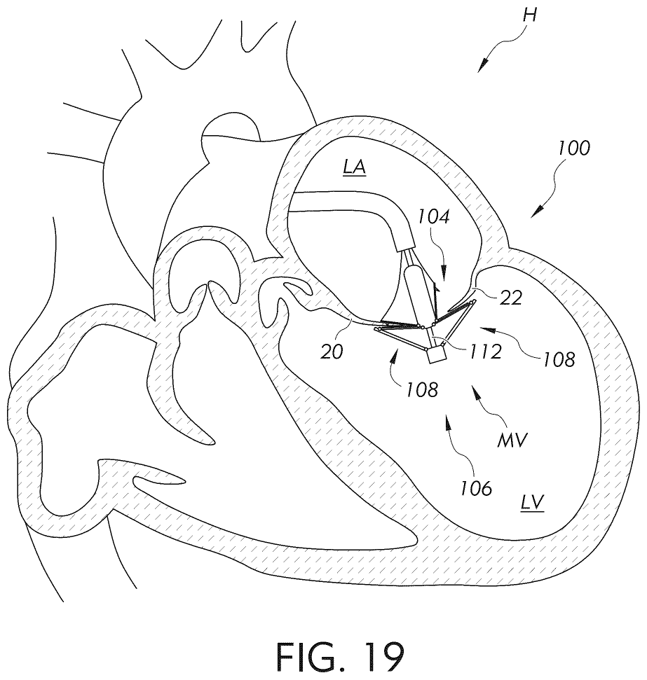

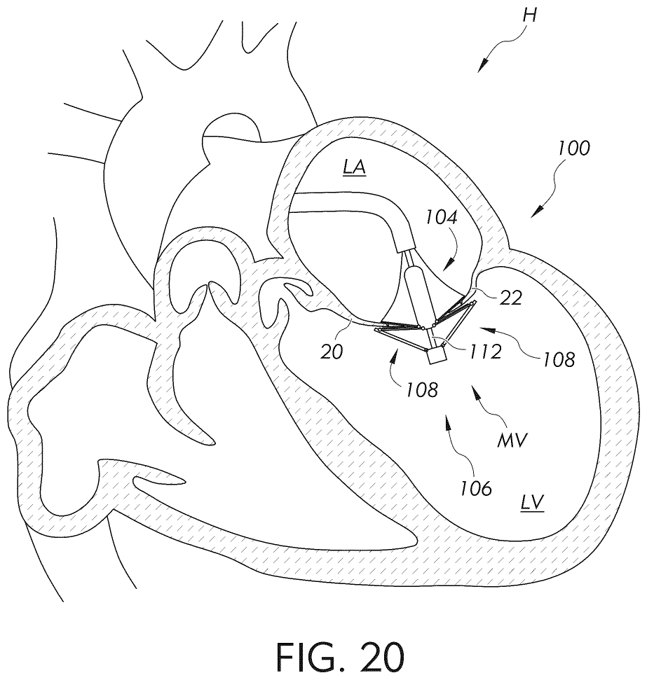

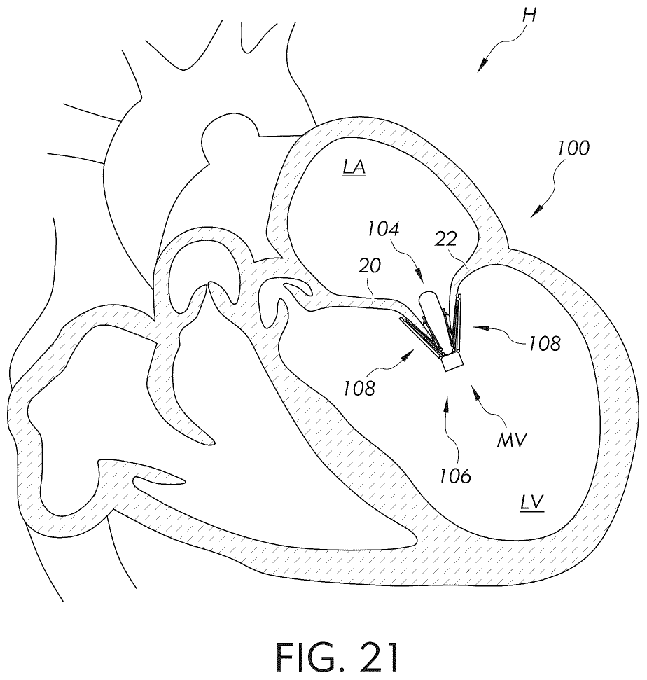

Referring now to FIG. 19, the device catheter can be retracted into the steerable catheter to position the mitral valve leaflets 20, 22 in the clasps 130. An actuation line 116 is extended to close one of the clasps 130, capturing a leaflet 20. FIG. 20 shows the other actuation line 116 being then extended to close the other clasp 130, capturing the remaining leaflet 22. Lastly, as can be seen in FIG. 21, the delivery system 102 (e.g., steerable catheter, implant catheter, etc.), actuation element 112 and actuation lines 116 are then retracted and the device 100 is fully closed and deployed in the native mitral valve MV.

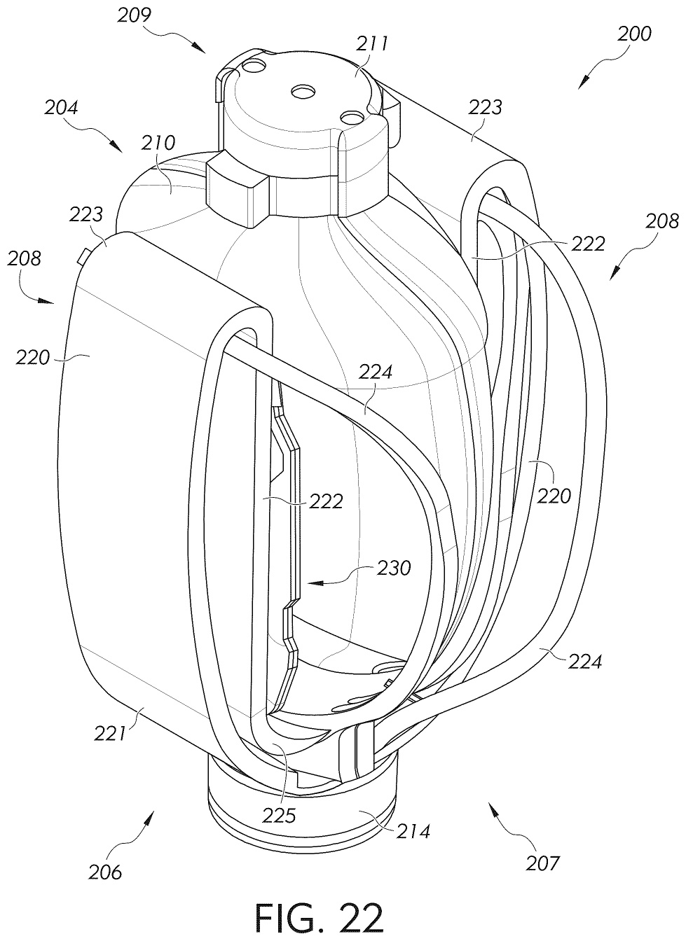

Any of the features disclosed by the present application can be used in a wide variety of different treatment devices and/or repair devices. FIGS. 22-24 illustrate examples of valve treatment and/or repair devices that can be modified to include any of the features disclosed by the present application. Any combination or sub-combination of the features disclosed by the present application can be combined with, substituted for, and/or added to any combination or sub-combination of the features of the devices illustrated by FIGS. 8-24.

Referring now to FIG. 22, an example of a device 200 (e.g., treatment device, repair device, implantable device, implant, etc.) is shown. The device 200 can be configured as an implantable device or implant or other valve treatment device (e.g., one that does not necessarily remain implanted). The device 200 is one of the many different configurations that the device 100 that is schematically illustrated in FIGS. 8-14 can take. The device 200 can include any other features for a device or implant discussed in the present application, and the device 200 can be positioned to engage valve tissue 20, 22 as part of any suitable treatment and/or repair system (e.g., any valve repair system and/or treatment system disclosed in the present application). The device/implant 200 can be a prosthetic spacer device, valve repair device, treatment device, or another type of implant that attaches to leaflets of a native valve.

In some implementations, the device 200 includes a coaptation portion 204, a proximal or attachment portion 209, an anchor portion 206, and a distal portion 207. In some implementations, the coaptation portion 204 of the device optionally includes a coaptation element 210 (e.g., a spacer, coaption element, plug, membrane, sheet, gap filler, plug, wedge, balloon, etc.) for deployment and/or implantation between leaflets of a native valve. In some implementations, the anchor portion 206 includes a plurality of anchors 208. The anchors can be configured in a variety of ways. In some implementations, each anchor 208 includes outer paddles 220, inner paddles 222, paddle extension members or paddle frames 224, and clasps 230. In some implementations, the attachment portion 209 includes a first or proximal collar 211 (or other attachment element) for engaging with a capture mechanism of a delivery system. A delivery system for the device 200 can be the same as or similar to delivery system 102 described above and can comprise one or more of a catheter, a sheath, a guide catheter/sheath, a delivery catheter/sheath, a steerable catheter, an implant catheter, a tube, a channel, a pathway, combinations of these, etc. The capture mechanism can be configured in a variety of ways and, in some implementations, can comprise one or more of a clamp, clip, pin, suture, line, lasso, noose, snare, buckle, lock, latch, etc.

In some implementations, the coaptation element 210 and paddles 220, 222 are formed from a flexible material that can be a metal fabric, such as a mesh, woven, braided, or formed in any other suitable way or a laser cut or otherwise cut flexible material. The material can be cloth, shape-memory alloy wire—such as Nitinol—to provide shape-setting capability, or any other flexible material suitable for deployment and/or implantation in the human body.

An actuation element (e.g., actuation wire, shaft, tube, hypotube, line, suture, braid, etc.) can extend from a delivery system (not shown) to engage and enable actuation of the device or implant 200. In some implementations, the actuation element extends through the proximal collar 211, and spacer or coaptation element 210 to engage a cap 214 of the distal portion 207. The actuation element can be configured to removably engage the cap 214 with a threaded connection, or the like, so that the actuation element can be disengaged and removed from the device 200 after implantation.

The coaptation element 210 extends from the proximal collar 211 (or other attachment element) to the inner paddles 222. In some implementations, the coaptation element 210 has a generally elongated and round shape, though other shapes and configurations are possible. In some implementations, the coaptation element 210 has an elliptical shape or cross-section when viewed from above and has a tapered shape or cross-section when seen from a front view and a round shape or cross-section when seen from a side view. A blend of these three geometries can result in the three-dimensional shape of the illustrated coaptation element 210 that achieves the benefits described herein. The round shape of the coaptation element 210 can also be seen, when viewed from above, to substantially follow or be close to the shape of the paddle frames 224.

The size and/or shape of the coaptation element 210 can be selected to minimize the number of implants that a single patient will require (preferably one), while at the same time maintaining low transvalvular gradients. In some implementations, the anterior-posterior distance at the top of the coaptation element is about 5 mm, and the medial-lateral distance of the coaptation element at its widest is about 10 mm. In some implementations, the overall geometry of the device 200 can be based on these two dimensions and the overall shape strategy described above. It should be readily apparent that the use of other anterior-posterior distance anterior-posterior distance and medial-lateral distance as starting points for the device will result in a device having different dimensions. Further, using other dimensions and the shape strategy described above will also result in a device having different dimensions.

In some implementations, the outer paddles 220 are jointably attached to the cap 214 of the distal portion 207 by connection portions 221 and to the inner paddles 222 by connection portions 223. The inner paddles 222 are jointably attached to the coaptation element by connection portions 225. In this manner, the anchors 208 are configured similar to legs in that the inner paddles 222 are like upper portions of the legs, the outer paddles 220 are like lower portions of the legs, and the connection portions 223 are like knee portions of the legs.

In some implementations, the inner paddles 222 are stiff, relatively stiff, rigid, have rigid portions and/or are stiffened by a stiffening member or a fixed portion of the clasps 230. The inner paddle 222, the outer paddle 220, and the coaptation element can all be interconnected as described herein.

In some implementations, the paddle frames 224 are attached to the cap 214 at the distal portion 207 and extend to the connection portions 223 between the inner and outer paddles 222, 220. In some implementations, the paddle frames 224 are formed of a material that is more rigid and stiff than the material forming the paddles 222, 220 so that the paddle frames 224 provide support for the paddles 222, 220.

The paddle frames 224 can provide additional pinching force between the inner paddles 222 and the coaptation element 210 and assist in wrapping the leaflets around the sides of the coaptation element 210. That is, the paddle frames 224 can be configured with a round three-dimensional shape extending from the cap 214 to the connection portions 223 of the anchors 208. The connections between the paddle frames 224, the outer and inner paddles 220, 222, the cap 214, and the coaptation element 210 can constrain each of these parts to the movements and positions described herein. In particular the connection portion 223 is constrained by its connection between the outer and inner paddles 220, 222 and by its connection to the paddle frame 224. Similarly, the paddle frame 224 is constrained by its attachment to the connection portion 223 (and thus the inner and outer paddles 222, 220) and to the cap 214.

The wide configuration of the paddle frames 224 provides increased surface area compared to the inner paddles 222 alone. The increased surface area can distribute the clamping force of the paddles 220 and paddle frames 224 against the native leaflets over a relatively larger surface of the native leaflets in order to further protect the native leaflet tissue.

Additional features of the device 200, modified versions of the device, delivery systems for the device, and methods for using the device and delivery system are disclosed by Patent Cooperation Treaty International Application No. PCT/US2018/028189 (International Publication No. WO 2018/195215) and the other applications incorporated herein. Any combination or sub-combination of the features disclosed by the present application can be combined with any combination or sub-combination of the features disclosed by Patent Cooperation Treaty International Application No. PCT/US2018/028189 (International Publication No. WO 2018/195215) and/or the other applications incorporated herein. Patent Cooperation Treaty International Application No. PCT/US2018/028189 (International Publication No. WO 2018/195215) is incorporated herein by reference in its entirety.

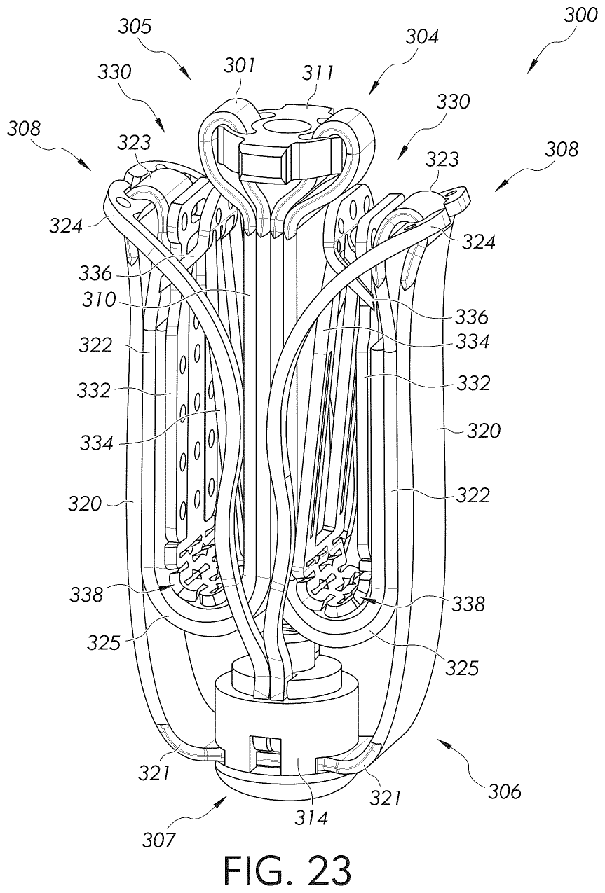

Referring now to FIG. 23, an example of a device 300 (e.g., a valve repair device, a valve treatment device, an implantable device, an implant, etc.) is shown. The device 300 is one of the many different configurations that the device 100 that is schematically illustrated in FIGS. 8-14 can take. The device 300 can include any other features for a device or implant discussed in the present application, and the device 300 can be positioned to engage valve tissue 20, 22 as part of any suitable treatment and/or repair system (e.g., any valve repair system and/or treatment system disclosed in the present application).

The device or implant 300 includes a proximal or attachment portion 305, an anchor portion 306, and a distal portion 307. In some implementations, the device/implant 300 includes a coaptation portion 304, and the coaptation portion 304 can optionally include a coaptation element 310 (e.g., spacer, plug, membrane, sheet, etc.) for deployment and/or implantation between the leaflets 20, 22 of the native valve. In some implementations, the anchor portion 306 includes a plurality of anchors 308. In some implementations, each anchor 308 can include one or more paddles, e.g., outer paddles 320, inner paddles 322, paddle extension members or paddle frames 324. The anchors can also include and/or be coupled to clasps 330. In some implementations, the attachment portion 305 includes a first or proximal collar 311 (or other attachment element) for engaging with a capture mechanism of a delivery system.

The anchors 308 can be attached to the other portions of the device and/or to each other in a variety of different ways (e.g., directly, indirectly, welding, sutures, adhesive, links, latches, integrally formed, a combination of some or all of these, etc.). In some implementations, the anchors 308 are attached to a coaptation element 310 by connection portions 325 and to a cap 314 by connection portions 321.

The anchors 308 can comprise first portions or outer paddles 320 and second portions or inner paddles 322 separated by connection portions 323. The connection portions 323 can be attached to paddle frames 324 that are hingeably attached to a cap 314 or other attachment portion. In this manner, the anchors 308 are configured similar to legs in that the inner paddles 322 are like upper portions of the legs, the outer paddles 320 are like lower portions of the legs, and the connection portions 323 are like knee portions of the legs.

In implementations with a coaptation element 310, the coaptation element 310 and the anchors 308 can be coupled together in various ways. As shown in the illustrated example, the coaptation element 310 and the anchors 308 can be coupled together by integrally forming the coaptation element 310 and the anchors 308 as a single, unitary component. This can be accomplished, for example, by forming the coaptation element 310 and the anchors 308 from a continuous strip 301 of a braided or woven material, such as braided or woven nitinol wire. In the illustrated example, the coaptation element 310, the outer paddle portions 320, the inner paddle portions 322, and the connection portions 321, 323, 325 are formed from a continuous strip 301.

Like the anchors 208 of the device 200 described above, the anchors 308 can be configured to move between various configurations by axially moving the distal end of the device (e.g., cap 314, etc.) relative to the proximal end of the device (e.g., proximal collar 311 or other attachment element, etc.). This movement can be along a longitudinal axis extending between the distal end (e.g., cap 314, etc.) and the proximal end (e.g., collar 311 or other attachment element, etc.) of the device.

In some implementations, in the straight configuration, the paddle portions 320, 322 are aligned or straight in the direction of the longitudinal axis of the device. In some implementations, the connection portions 323 of the anchors 308 are adjacent the longitudinal axis of the spacer or coaptation element 310. From the straight configuration, the anchors 308 can be moved to a fully folded configuration (e.g., FIG. 23), e.g., by moving the proximal end and distal end toward each other and/or toward a midpoint or center of the device.

In some implementations, the clasps comprise a moveable arm coupled to an anchor. In some implementations, the clasps 330 include a base or fixed arm 332, a moveable arm 334, optional barbs/friction-enhancing elements 336, and a joint portion 338. The fixed arms 332 are attached to the inner paddles 322, with the joint portion 338 disposed proximate the coaptation element 310. The joint portion 338 is spring-loaded so that the fixed and moveable arms 332, 334 are biased toward each other when the clasp 330 is in a closed condition.

The fixed arms 332 are attached to the inner paddles 322 through holes or slots with sutures. The fixed arms 332 can be attached to the inner paddles 322 with any suitable means, such as screws or other fasteners, crimped sleeves, mechanical latches or snaps, welding, adhesive, or the like. The fixed arms 332 remain substantially stationary relative to the inner paddles 322 when the moveable arms 334 are opened to open the clasps 330 and expose the optional barbs 336. The clasps 330 are opened by applying tension to actuation lines attached to the moveable arms 334, thereby causing the moveable arms 334 to articulate, pivot, and/or flex on the joint portions 338.

In short, the device 300 is similar in configuration and operation to the device 200 described above, except that the coaptation element 310, outer paddles 320, inner paddles 322, and connection portions 321, 323, 325 are formed from the single strip of material 301. In some implementations, the strip of material 301 is attached to the proximal collar 311, cap 314, and paddle frames 324 by being woven or inserted through openings in the proximal collar 311, cap 314, and paddle frames 324 that are configured to receive the continuous strip of material 301. The continuous strip 301 can be a single layer of material or can include two or more layers. In some implementations, portions of the device 300 have a single layer of the strip of material 301 and other portions are formed from multiple overlapping or overlying layers of the strip of material 301.

For example, FIG. 23 shows a coaptation element 310 and inner paddles 322 formed from multiple overlapping layers of the strip of material 301. The single continuous strip of material 301 can start and end in various locations of the device 300. The ends of the strip of material 301 can be in the same location or different locations of the device 300. For example, in the illustrated example of FIG. 23, the strip of material 301 begins and ends in the location of the inner paddles 322.

As with the device 200 described above, the size of the coaptation element 310 can be selected to minimize the number of implants that a single patient will require (preferably one), while at the same time maintaining low transvalvular gradients. In particular, forming many components of the device 300 from the strip of material 301 allows the device 300 to be made smaller than the device 200. For example, in some implementations, the anterior-posterior distance at the top of the coaptation element 310 is less than 2 mm, and the medial-lateral distance of the device 300 (i.e., the width of the paddle frames 324 which are wider than the coaptation element 310) at its widest is about 5 mm.

Additional features of the device 300, modified versions of the device, delivery systems for the device, and methods for using the device and delivery system are disclosed by Patent Cooperation Treaty International Application No. PCT/US2019/055320 (International Publication No. WO 2020/076898) and/or any other applications incorporated herein. Any combination or sub-combination of the features disclosed by the present application can be combined with any combination or sub-combination of the features disclosed by Patent Cooperation Treaty International Application No. PCT/US2019/055320 (International Publication No. WO 2020/076898) and/or any other applications incorporated herein. Patent Cooperation Treaty International Application No. PCT/US2019/055320 (International Publication No. WO 2020/076898) is incorporated herein by reference in its entirety.

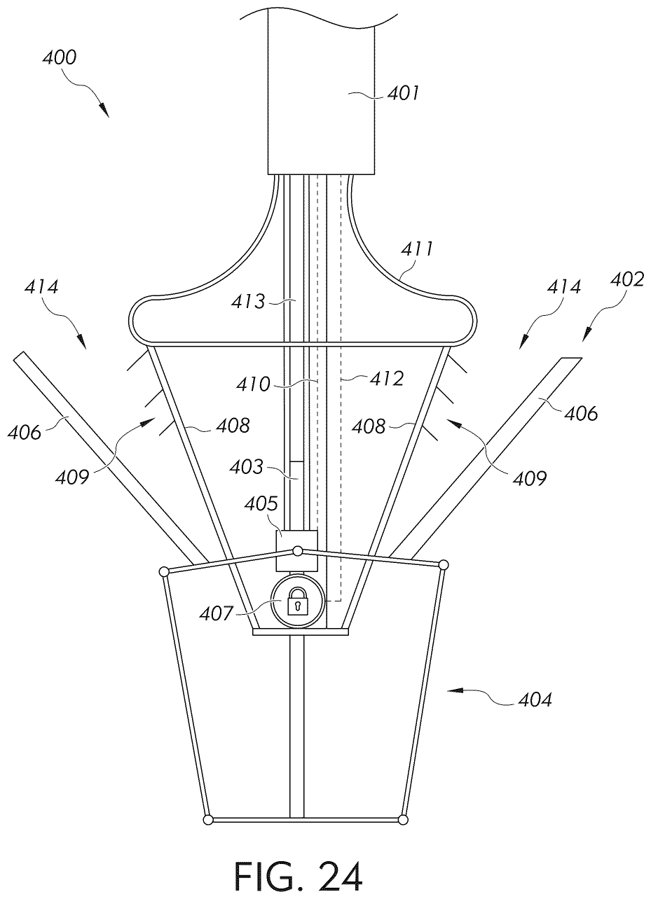

FIG. 24 illustrates an example of one of the many treatment and/or repair systems 400 for treating and/or repairing a native valve of a patient that the concepts of the present application can be applied to. The treatment and/or repair system 400 includes a delivery device 401 and a treatment and/or repair device 402.

In some implementations, the treatment device or repair device 402 includes a base assembly 404, a pair of paddles 406, and a pair of gripping members 408 (e.g., clasps, clasp arms, grippers, gripping arms, latches, etc.). In one example, the paddles 406 can be integrally formed with the base assembly. For example, the paddles 406 can be formed as extensions of links of the base assembly. In the illustrated example, the base assembly 404 of the device 402 has a shaft 403, a coupler 405 configured to move along the shaft, and a lock 407 configured to lock the coupler in a stationary position on the shaft. The coupler 405 is mechanically connected to the paddles 406, such that movement of the coupler 405 along the shaft 403 causes the paddles to move between an open position and a closed position. In this way, the coupler 405 serves as a means for mechanically coupling the paddles 406 to the shaft 403 and, when moving along the shaft 403, for causing the paddles 406 to move between their open and closed positions.

In some implementations, the gripping members 408 are pivotally connected to the base assembly 404 (e.g., the gripping members 408 can be pivotally connected to the shaft 403, or any other suitable member of the base assembly), such that the gripping members can be moved to adjust the width of the opening 414 between the paddles 406 and the gripping members 408. The gripping member 408 can include an optional barbed portion 409 for attaching the gripping members to valve tissue when the device 402 is attached to the valve tissue. When the paddles 406 are in the closed position, the paddles engage the gripping members 408, such that, when valve tissue is attached to the barbed portion 409 of the gripping members, the paddles secure the device 402 to the valve tissue. In some implementations, the gripping members 408 are configured to engage the paddles 406 such that the barbed portion 409 engages the valve tissue member and the paddles 406 to secure the device 402 to the valve tissue member. For example, in certain situations, it can be advantageous to have the paddles 406 maintain an open position and have the gripping members 408 move outward toward the paddles 406 to engage valve tissue and the paddles 406.

While the example shown in FIG. 24 illustrates a pair of paddles 406 and a pair of gripping members 408, it should be understood that the device 402 can include any suitable number of paddles and gripping members.

In some implementations, the system 400 includes a placement shaft 413 that is removably attached to the shaft 403 of the base assembly 404 of the device 402. In some implementations, after the device 402 is secured to valve tissue, the placement shaft 413 can be removed from the shaft 403 to remove the device 402 from the remainder of the treatment and/or repair system 400, such that the device 402 can remain attached to the valve tissue, and the delivery device 401 can be removed from a patient's body.

The treatment and/or repair system 400 can also include a paddle control mechanism 410, a gripper control mechanism 411, and a lock control mechanism 412. The paddle control mechanism 410 is mechanically attached to the coupler 405 to move the coupler along the shaft, which causes the paddles 406 to move between the open and closed positions. The paddle control mechanism 410 can take any suitable form, and can comprise, for example, a shaft, wire, tube, hypotube, rod, suture, line, etc. For example, the paddle control mechanism can comprise a hollow shaft, a catheter tube or a sleeve that fits over the placement shaft 413 and the shaft 403 and is connected to the coupler 405.

The gripper control mechanism 411 is configured to move the gripping members 408 such that the width of the opening 414 between the gripping members and the paddles 406 can be altered. The gripper control mechanism 411 can take any suitable form, such as, for example, a line, a suture or wire, a rod, a catheter, a tube, a hypotube, etc.

The lock control mechanism 412 is configured to lock and unlock the lock. The lock 407 locks the coupler 405 in a stationary position with respect to the shaft 403 and can take a wide variety of different forms and the type of lock control mechanism 412 can be dictated by the type of lock used. In examples in which the lock 407 includes a pivotable plate, the lock control mechanism 412 is configured to engage the pivotable plate to move the plate between the tilted and substantially non-tilted positions. The lock control mechanism 412 can be, for example, a rod, a suture, a wire, or any other member that is capable of moving a pivotable plate of the lock 407 between a tilted and substantially non-tilted position.

The device 402 is movable from an open position to a closed position. The base assembly 404 includes links that are moved by the coupler 405. The coupler 405 is movably attached to the shaft 403. In order to move the device from the open position to the closed position, the coupler 405 is moved along the shaft 403, which moves the links.

The gripper control mechanism 411 is moves the gripping members 408 to provide a wider or a narrower gap at the opening 414 between the gripping members and the paddles 406. In the illustrated example, the gripper control mechanism 411 includes a line, such as a suture, a wire, etc. that is connected to an opening in an end of the gripping members 408. When the line(s) is pulled, the gripping members 408 move inward, which causes the opening 414 between the gripping members and the paddles 406 to become wider.

In order to move the device 402 from the open position to the closed position, the lock 407 is moved to an unlocked condition by the lock control mechanism 412. Once the lock 407 is in the unlocked condition, the coupler 405 can be moved along the shaft 403 by the paddle control mechanism 410.

After the paddles 406 are moved to the closed position, the lock 407 is moved to the locked condition by the lock control mechanism 412 to maintain the device 402 in the closed position. After the device 402 is maintained in the locked condition by the lock 407, the device 402 is removed from the delivery device 401 by disconnecting the shaft 403 from the placement shaft 413. In addition, the device 402 is disengaged from the paddle control mechanism 410, the gripper control mechanism 411, and the lock control mechanism 412.

Additional features of the device 402, modified versions of the device, delivery systems for the device, and methods for using the device and delivery system are disclosed by Patent Cooperation Treaty International Application No. PCT/US2019/012707 (International Publication No. WO 2019139904) and/or any other applications incorporated herein. Any combination or sub-combination of the features disclosed by the present application can be combined with any combination or sub-combination of the features disclosed by Patent Cooperation Treaty International Application No. PCT/US2019/012707 (International Publication No. WO 2019139904) and/or any other applications incorporated herein. Patent Cooperation Treaty International Application No. PCT/US2019/012707 (International Publication No. WO 2019139904) is incorporated herein by reference in its entirety.

Clasps or leaflet gripping devices disclosed herein can take a wide variety of different forms. Examples of clasps are disclosed by Patent Cooperation Treaty International Application No. PCT/US2018/028171 (International Publication No. WO 2018195201). Any combination or sub-combination of the features disclosed by the present application can be combined with any combination or sub-combination of the features disclosed by Patent Cooperation Treaty International Application No. PCT/US2018/028171 (International Publication No. WO 2018195201). Patent Cooperation Treaty International Application No. PCT/US2018/028171 (International Publication No. WO 2018195201) is incorporated herein by reference in its entirety.

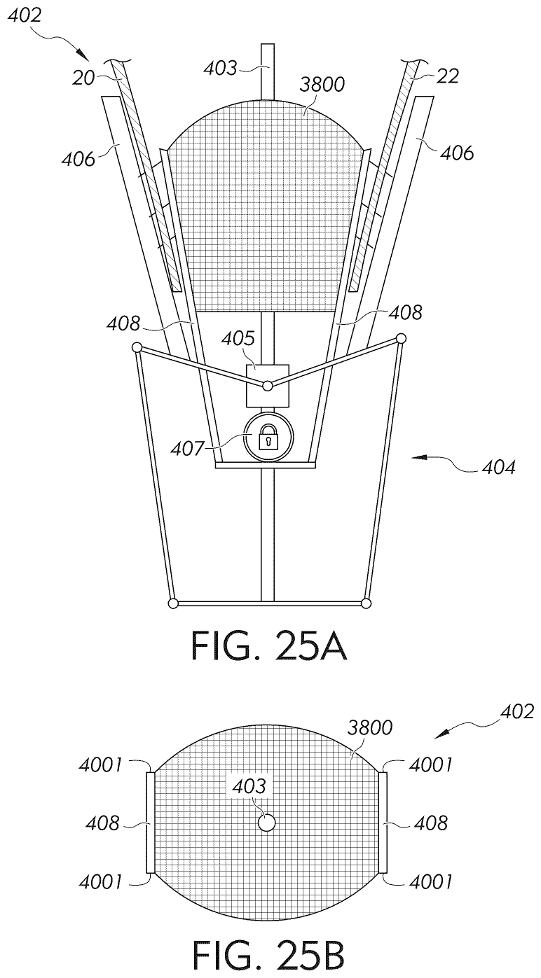

Referring to FIGS. 25A-25B, an example implementation of a treatment and/or repair device 402 has a coaptation element 3800. The device 402 can have the same configuration as the device illustrated by FIG. 24 with the addition of the coaptation element. The coaptation element 3800 can take a wide variety of different forms. The coaptation element 3800 can be compressible and/or expandable. For example, the coaptation element can be compressed to fit inside one or more catheters of a delivery system, can expand when moved out of the one or more catheters, and/or can be compressed by the paddles 406 to adjust the size of the coaptation element. In the example illustrated by FIGS. 25A and 25B, the size of the coaptation element 3800 can be reduced by squeezing the coaptation element with the paddles 406 and can be increased by moving the paddles 406 away from one another. The coaptation element 3800 can extend past outer edges 4001 of the gripping members or clasps 408 as illustrated for providing additional surface area for closing the gap of a mitral valve.

The coaptation element 3800 can be coupled to the device 402 in a variety of different ways. For example, the coaptation element 3800 can be fixed to the shaft 403, can be slidably disposed around the shaft, can be connected to the coupler 405, can be connected to the lock 407, and/or can be connected to a central portion of the clasps or gripping members 408. In some implementations, the coupler 405 can take the form of the coaptation element 3800. That is, a single element can be used as the coupler 405 that causes the paddles 406 to move between the open and closed positions and the coaptation element 3800 that closes the gap between the leaflets 20, 22 when the device 402 is attached to the leaflets.

The coaptation element 3800 can be disposed around one or more of the shafts or other control elements of the system 400. For example, the coaptation element 3800 can be disposed around the shaft 403, the shaft 413, the paddle control mechanism 410, and/or the lock control mechanism 412.

The device 402 can include any other features for a device, treatment device, repair device, implant, etc. discussed in the present application, and the device 402 can be positioned to engage valve tissue as part of any suitable treatment and/or repair system (e.g., any valve repair system and/or treatment system disclosed in the present application). Additional features of the device 402, modified versions of the device, delivery systems for the device, and methods for using the device and delivery system are disclosed by Patent Cooperation Treaty International Application No. PCT/US2019/012707 (International Publication No. WO 2019139904). Any combination or sub-combination of the features disclosed by the present application can be combined with any combination or sub-combination of the features disclosed by Patent Cooperation Treaty International Application No. PCT/US2019/012707 (International Publication No. WO 2019139904).

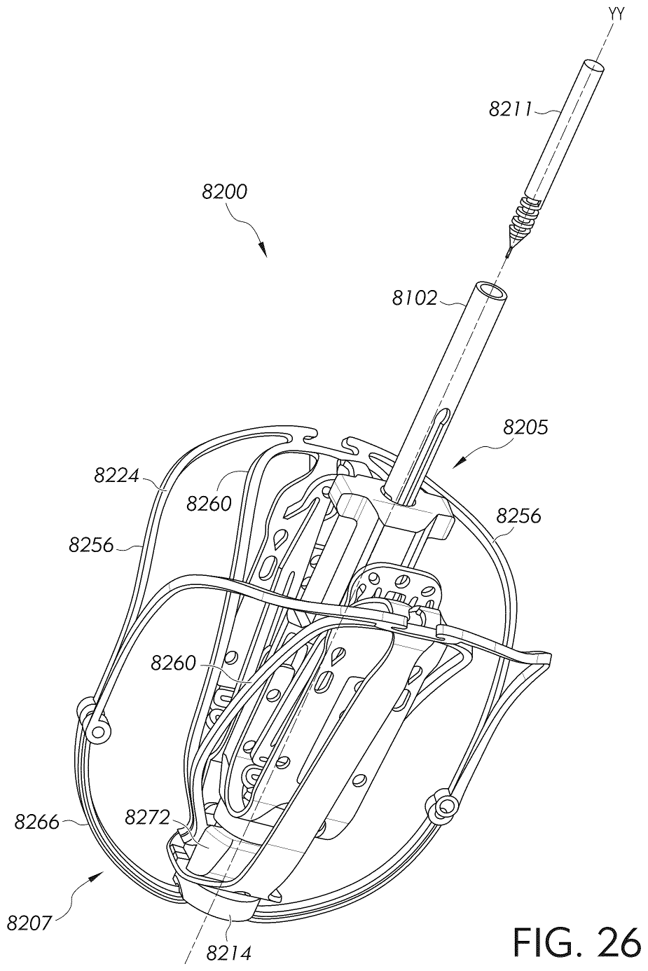

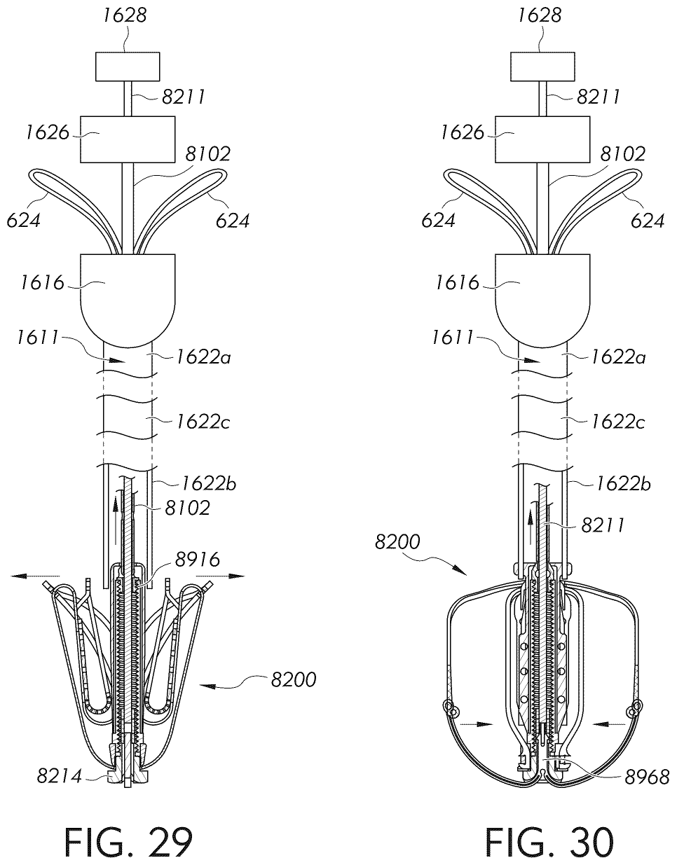

FIGS. 26-30 illustrate an example of one of the many systems for treating and/or repairing a native valve of a patient that the concepts of the present application can be applied to. Referring to FIGS. 29 and 30, the system includes a catheter assembly 1611 (e.g., a device catheter assembly, an implant catheter assembly, treatment catheter assembly, etc.) and a treatment and/or repair device 8200. Referring to FIGS. 26-28, the device 8200 includes a proximal or attachment portion 8205, paddle frames 8224, and a distal portion 8207. The attachment portion 8205, the distal portion 8207, and the paddle frames 8224 can be configured in a variety of ways.

In the example illustrated in FIG. 26, the paddle frames 8224 can be symmetric along longitudinal axis YY. However, in some implementations, the paddle frames 8224 are not symmetric about the axis YY. Moreover, referring to FIG. 26, the paddle frames 8224 include outer frame portions 8256 and inner frame portions 8260.

In some implementations, the connector 8266 (e.g., shaped metal component, shaped plastic component, tether, wire, strut, line, cord, suture, etc.) attaches to the outer frame portions 8256 at outer ends of the connector 8266 and to a coupler 8972 at an inner end 8968 of the connector 8266 (see FIG. 28). Between the connector 8266 and the attachment portion 8205, the outer frame portions 8256 form a curved shape. For example, in the illustrated example, the shape of the outer frame portions 8256 resembles an apple shape in which the outer frame portions 8256 are wider toward the attachment portion 8205 and narrower toward the distal portion 8207. In some implementations, however, the outer frame portions 8256 can be otherwise shaped.

The inner frame portions 8260 extend from the attachment portion 8205 toward the distal portion 8207. The inner frame portions 8260 then extend inward to form retaining portions 8272 that are attached to the actuation cap 8214. The retaining portions 8272 and the actuation cap 8214 can be configured to attach in any suitable manner.

In some implementations, the inner frame portions 8260 are rigid frame portions, while the outer frame portions 8256 are flexible frame portions. The proximal end of the outer frame portions 8256 connect to the proximal end of the inner frame portions 8260, as illustrated in FIG. 26.

The width adjustment element 8211 (e.g., width adjustment wire, width adjustment shaft, width adjustment tube, width adjustment line, width adjustment cord, width adjustment suture, width adjustment screw or bolt, etc.) is configured to move the outer frame portions 8256 from the expanded position to the narrowed position by pulling the inner end 8968 (FIG. 28) and portions of the connector 8266 into the actuation cap 8214. The actuation element 8102 is configured to move the inner frame portions 8260 to open and close the paddles in accordance with some implementations disclosed herein.

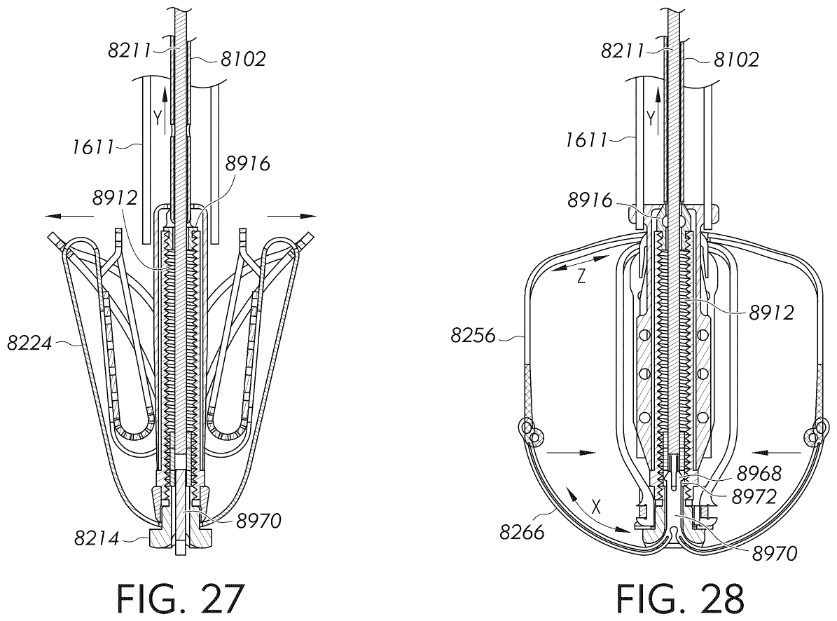

As shown in FIGS. 27 and 28, the connector 8266 has an inner end 8968 that engages with the width adjustment element 8211 such that a user can move the inner end 8968 inside the receiver 8912 (e.g., an internally threaded element, a column, a conduit, a hollow member, a notched receiving portion, a tube, a shaft, a sleeve, a post, a housing, a cylinder, tracks, etc.) to move the outer frame portions 8256 between a narrowed position and an expanded position. In the illustrated example, the inner end 8968 includes a post 8970 that attaches to the outer frame portions 8256 and a coupler 8972 that extends from the post 8970. The coupler 8972 is configured to attach and detach from both the width adjustment element 8211 and the receiver 8912. The coupler 8972 can take a wide variety of different forms. For example, the coupler 8972 can include one or more of a threaded connection, features that mate with threads, detent connections, such as outwardly biased arms, walls or other portions. When the coupler 8972 is attached to the width adjustment element 8211, the coupler is released from the receiver 8912. When the coupler 8972 is detached from the width adjustment element 8211, the coupler is secured to the receiver. The inner end 8968 of the connector can, however, be configured in a variety of ways. Any configuration that can suitably attach the outer frame portions 8256 to the coupler to allow the width adjustment element 8211 to move the outer frame portions 8256 between the narrowed position and the expanded position can be used. The coupler can be configured in a variety of ways as well and can be a separate component or be integral with another portion of the device, e.g., of the connector or inner end of the connector.

The width adjustment element 8211 allows a user to expand or contract the outer frame portions 8256 of the device 8200. In the example illustrated in FIGS. 27 and 28, the width adjustment element 8211 includes an externally threaded end that is threaded into the coupler 8972. The width adjustment element 8211 moves the coupler in the receiver 8912 to adjust the width of the outer frame portions 8256. When the width adjustment element 8211 is unscrewed from the coupler 8972, the coupler engages the inner surface of the receiver 8912 to set the width of the outer frame portions 8256.

In some implementations, the receiver 8912 can be integrally formed with a distal cap 8214. Moving the cap 8214 relative to a body of the attachment portion 8205 opens and closes the paddles. In the illustrated example, the receiver 8912 slides inside the body of the attachment portion. When the coupler 8972 is detached from the width adjustment element 8211, the width of the outer frame portions 8256 is fixed while the actuation element 8102 moves the receiver 8912 and cap 8214 relative to a body of the attachment portion 8205. Movement of the cap can open and close the device in the same manner as some of the examples disclosed above.

In the illustrated example, a driver head 8916 is disposed at a proximal end of the actuation element 8102. The driver head 8916 releasably couples the actuation element 8102 to the receiver 8912. In the illustrated example, the width adjustment element 8211 extends through the actuation element 8102. The actuation element is axially advanced in the direction opposite to direction Y to move the distal cap 8214. Movement of the distal cap 8214 relative to the attachment portion 8205 is effective to open and close the paddles, as indicated by the arrows in FIG. 27. That is, movement of the distal cap 8214 in the direction Y closes the device and movement of the distal cap in the direction opposite to direction Y opens the device.

Also illustrated in FIGS. 27 and 28, the width adjustment element 8211 extends through the actuation element 8102, the driver head 8916, and the receiver 8912 to engage the coupler 8972 attached to the inner end 8968. The movement of the outer frame portions 8256 to the narrowed position can allow the device or implant 8200 to maneuver more easily into position for deployment and/or implantation in the heart by reducing the contact and/or friction between the native structures of the heart—e.g., chordae—and the device 8200. The movement of the outer frame portions 8256 to the expanded position provides the anchor portion of the device 8200 with a larger surface area to engage and capture leaflet(s) of a native heart valve.

Referring to FIGS. 29 and 30, an example of a catheter assembly 1611 (e.g., a device catheter assembly, an implant catheter assembly, treatment catheter assembly, etc.) in which clasp actuation lines 624 extend through a handle 1616, the actuation element 8102 is coupled to a paddle actuation control 1626, and the width adjustment element 8211 is coupled to a paddle width control 1628. A proximal end portion 1622a of the shaft or catheter of the catheter assembly 1611 can be coupled to the handle 1616, and a distal end portion 1622b of the shaft or catheter can be coupled to the device 8200. The actuation element 8102 can extend distally from the paddle actuation control 1626, through the handle 1616, through the delivery shaft or catheter of the catheter assembly 1611, and through the proximal end of the device 8200, where it couples with the driver head 8916. The actuation element 8102 can be axially movable relative to the outer shaft of the catheter assembly 1611 and the handle 1616 to open and close the device.

The width adjustment element 8211 can extend distally from the paddle width control 1628, through the paddle actuation control 1626 and through the actuation element 8102 (and, consequently, through the handle 1616, the outer shaft of the implant catheter assembly 1611, and through the device 8200), where it couples with the movable coupler 8972. The width adjustment element 8211 can be axially movable relative to the actuation element 8102, the outer shaft of the implant catheter assembly 1611, and the handle 1616. The clasp actuation lines 624 can extend through and be axially movable relative to the handle 1616 and the outer shaft of the implant catheter assembly 1611. The clasp actuation lines 624 can also be axially movable relative to the actuation element 8102.

Referring to FIGS. 29 and 30, the width adjustment element 8211 can be releasably coupled to the coupler 8972 of the device 8200. Advancing and retracting the width adjustment element 8211 with the paddle width control 1628 widens and narrows the paddles. Advancing and retracting the actuation element 8102 with the paddle actuation control 1626 opens and closes the paddles of the device.

In the examples of FIGS. 29 and 30, the catheter or shaft of the catheter assembly 1611 is an elongate shaft extending axially between the proximal end portion 1622a, which is coupled to the handle 1616, and the distal end portion 1622b, which is coupled to the device 8200. The outer shaft of the catheter assembly 1611 can also include an intermediate portion 1622c disposed between the proximal and distal end portions 1622a, 1622b.

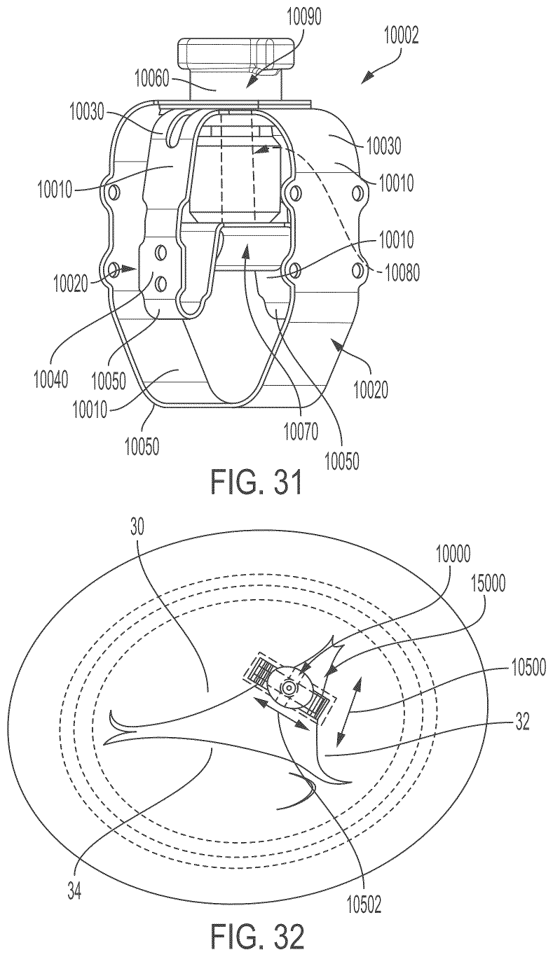

In some implementations, as shown in FIG. 31, an example expandable mechanism 10002 can be formed from one or more struts 10010. In some implementations, the struts 10010 have a plurality of rigid portions 10020 that are connected together at flexible proximal connection regions 10030 and flexible distal connection regions 10050. In some implementations, the flexible proximal connection regions 10030 and flexible distal connection regions 10050 enable the rigid portions 10020 to move relative to one another.

In some implementations, the connection regions 10030, 10050 can be solid pieces of the same material that makes up the rigid portions 10020. In some implementations, the flexible connection regions 10030, 10050 can be connected via interconnecting couplers or other means for connection.

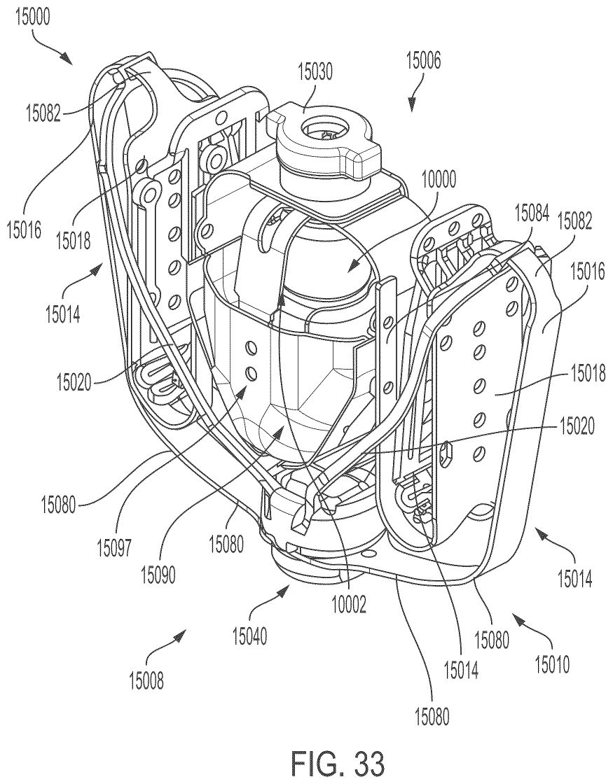

The expandable mechanism 10002 can be used in a variety of devices, implants, valve repair devices, e.g., device 15000 illustrated in FIGS. 33-39 and/or other devices disclosed herein.

The rigid portions 10020 can include an attachment region 10040 for attaching another component of the expandable coaptation element 10000. For example, shell components 15090 and/or a sleeve 17000, which are described below can be attached to the attachment region 10040. The shell components 15090 and/or a sleeve 17000 can provide the expandable coaptation element 10000 with a substantially continuous and/or smooth outer surface.

Some or all of the struts 10010 are movable between a collapsed condition and an expanded condition. The struts can be moved between the collapsed condition and the expanded condition in a variety of different ways. In the example illustrated by FIG. 31, one pair of the struts 10010 are moved between the collapsed condition and an expanded condition via a screw 10080 and a nut 10090. Adjusting the nut 10090 extends or draws the screw 10080, which will expand and/or contract the struts 10010.

In some implementations, the screw 10080 extends through a shaft 10060 and extends to an actuation member 10070. In the example illustrated by FIG. 31, advancing the screw through the nut moves the actuation member 10070 distally. In this example, the distal movement of the actuation member 10070 forces the attachment region 10040 outward to increase the width of the expandable mechanism 10002. Conversely, retracting the screw through the nut moves the actuation member 10070 proximally. The proximal movement of the actuation member 10070 moves the attachment regions 10040 inward to decrease the width of the expandable mechanism 10002.

In the example illustrated by FIG. 31, The expandable mechanism 10002 expands in one direction, since only one pair of struts 10010 are coupled to the actuation member 10070. However, in some implementations, both pairs or different arrangements of struts can be coupled to one or more actuation members such that the expandable mechanism can expand in more than one direction.

In some implementations, adjusting the nut 10090 and screw 10080 relative to one another can expand and contract two pairs of struts 10010 of the expandable mechanism 10002 in the same direction (i.e., both expand or both contract). In some implementations, adjusting the nut 10090 relative to the screw 10080 can expand and contract the two pairs of struts 10010 of the expandable mechanism 10002 in opposite directions (i.e., one pair expands while the other contracts). The widths of both pairs of struts 10010 can be adjusted by a single adjustment mechanism (e.g., actuation member 10070, screw 10080, and nut 10090) or two independent adjustment mechanisms can be used to independently adjust each of the pairs of struts. Control can be achieved individually, simultaneously, sequentially, or otherwise.

The rigid portions 10020 and the flexible portions 10050 of the struts 10010 can include an optional cover over the struts 10010. The struts 10010 can include shaped portions and/or can include flexible or pliable portions to allow the shape of the expandable mechanism 10002 to conform to the shape of the native leaflets 20, 22 when the leaflets 20, 22 close against the expandable coaptation element 10000. This can improve engagement between the expandable coaptation element 10000 and the native leaflets 20, 22. While four struts 10010 are shown, any number of struts 10010 can be combined to provide expanding and contracting coaptation surfaces. Different struts can include rigid portions 10020 having different shapes and lengths to provide differing rates of expansion and contraction and different maximum expansion positions to form a wide variety of differently shaped expandable coaptation elements 10000.

FIG. 32 shows an expandable coaptation element 10000 of a device 15000 (e.g., a treatment device, a repair device, an implantable device, an implant, etc.) implemented between leaflets of a native valve, such as the illustrated tricuspid valve or a native mitral valve. Once the device is deployed as described above, the size of the expandable coaptation element 10000 can be adjusted in the direction 10500 and/or the direction 10502. The size of the expandable coaptation element 10000 can be adjusted based on regurgitation through the native valve during ventricular systole and/or flow through the valve during ventricular diastole. For example, the size of the expandable coaptation element 10000 can be adjusted to provide the largest reduction of regurgitation that allows the native valve to provide an acceptable flow from the atrium to the ventricle. In some implementations, the direction 10500 is a gap width of a septal-lateral annulus and the direction 10502 is along a gap length of an anterior-posterior annulus.

FIGS. 33-39 illustrate an example of a device 15000 (e.g., valve repair device, valve treatment device, implantable device, implant, etc.) having an expandable coaptation element 10000. The expandable coaptation element 10000 can be used in a variety of different devices, including but not limited to, any of the treatment and/or repair devices disclosed in the present application. For example, any expandable coaptation element disclosed herein can be used in the device that is schematically illustrated in FIGS. 8-14.

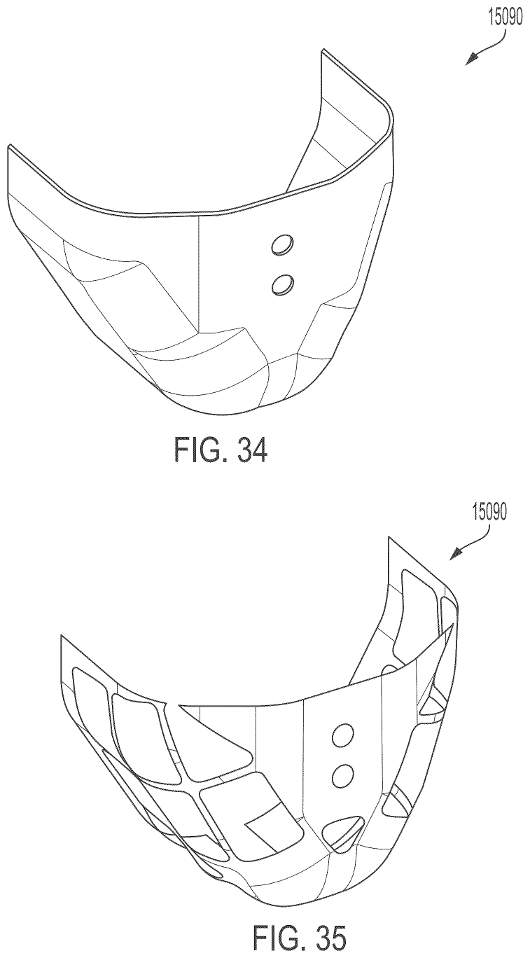

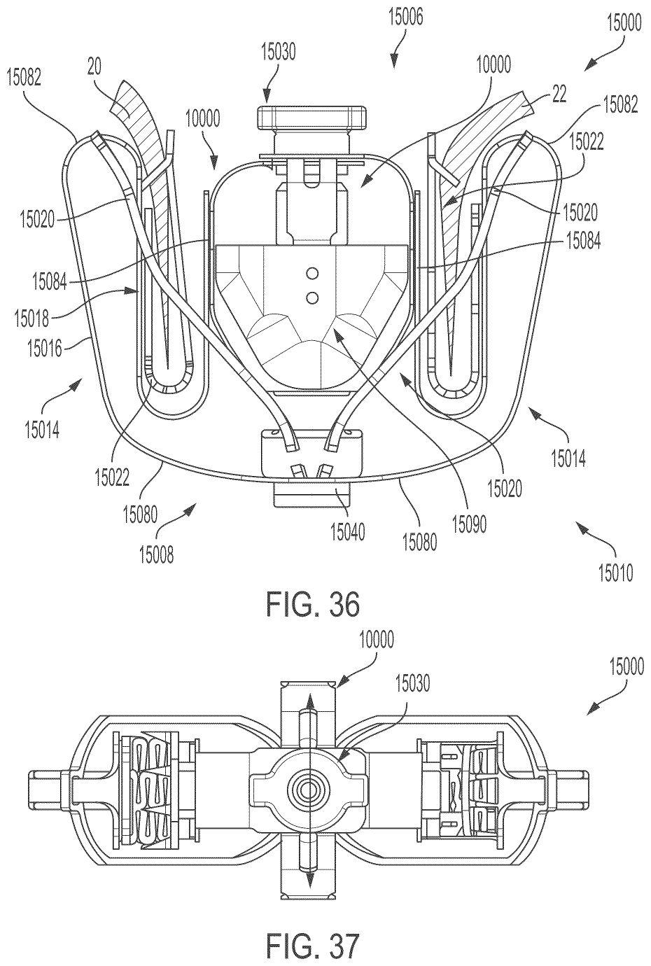

The device 15000 (See FIG. 33) is one of the many different configurations that the device that is schematically illustrated in FIGS. 8-14 with an expandable coaptation element 10000 can take. The device 15000 can include any other features for a device discussed in the present application, and the expandable coaptation element 10000 can be positioned to engage leaflets 30, 32, 34 (see FIGS. 7 and 34) or leaflets 20, 22 (see FIGS. 6 and 36) as part of any suitable device (e.g., any treatment and/or repair device disclosed in the present application).

The device 15000 can be deployed from a delivery sheath, a device catheter, and/or an implant catheter. The device 15000 can include an expandable mechanism 10002 (the expandable mechanism can be the same as or similar to any of the expandable mechanisms described anywhere in this disclosure) and an anchor portion having one or more anchors (which can be the same as or similar to any anchor portion and/or anchors described anywhere in this disclosure). The expandable mechanism can comprise one or more of a balloon, expandable container, expandable material, self-expanding material, self-expanding frame, stent, mechanically-expandable frame, pivoting and/or scissoring extensions and/or struts, expanding pully mechanism, Hoberman mechanism, cam, worm screw, rack and pinion, foam, etc.

The device 15000 can be a prosthetic spacer device, valve repair device, valve treatment device, implant, or another type of device that attaches to leaflets of a native valve.

Referring now to FIG. 33, a device 15000 (e.g., a valve repair device, an implantable device, implant, valve treatment device, etc.) with an expandable coaptation element 10000 is shown. The implantable device 15000 is one of many different configurations that the device 100 that is schematically illustrated in FIGS. 8-14 with an expandable coaptation element added can take.

In some implementations, the device 15000 includes an expandable coaptation element 10000, a proximal or attachment portion 15006, an anchor portion 15008, and a distal portion 15010. In some implementations, the expandable coaptation element 10000 is configured for adjustable implantation between leaflets of a native valve. In some implementations, the anchor portion 15008 includes a plurality of anchors 15014. The anchors can be configured in a variety of ways. In some implementations, each of the anchors 15014 includes outer paddles 15016, inner paddles 15018, paddle extension members or paddle frames 15020, and clasps 15022. In some implementations, the attachment portion 15006 includes a first or proximal collar 15030 (or other attachment element) for engaging with a capture mechanism of a delivery system. A delivery system for the device or implant 15000 can be the same as or similar to the delivery system 102 described above and can comprise one or more of a catheter, a sheath, a guide catheter/sheath, a delivery catheter/sheath, a steerable catheter, an implant catheter, a tube, a channel, a pathway, combinations of these, etc.

In some implementations, portions or components of the expandable coaptation element 10000 and/or the outer and inner paddles 15016, 15018 are formed from a flexible material that can be a metal fabric, such as mesh, woven, braided, or formed in any other suitable way or a laser cut or otherwise cut flexible material. The material can be cloth, shape-memory alloy wire—such as Nitinol—to provide shape-setting capability, or any other flexible material suitable for implantation in the human body.

An actuation element (e.g., actuation shaft, actuation rod, actuation tube, actuation wire, actuation line, etc.) can extend from a delivery system (not shown) to engage and enable actuation of the device 15000 (see actuation element 112 in FIGS. 8-14). In some implementations, the actuation element extends through the proximal collar 15030 and expandable coaptation element 10000 to engage a cap 15040 of the distal portion 15010. The actuation element can be configured to removably engage the cap 15040 with a threaded connection, or the like, so that the actuation element can be disengaged and removed from the implant 15000 after implantation.

The expandable coaptation element 10000 extends from the proximal collar 15030 (or other attachment) to the cap 15040. In some implementations, the expandable coaptation element 10000 has a generally elongated and round shape, though other shapes and configurations are possible. In some implementations, the expandable coaptation element 10000 has an elliptical shape or cross-section when viewed from above and has a tapered shape or cross-section when seen from a front view and a rounded shape or cross-section when seen from a side view. A blend of these three geometries can result in the three-dimensional shape of the illustrated expandable coaptation element 10000.

The size and/or shape of the expandable coaptation element 10000 can be selected and/or be adjusted to minimize the number of implants that a single patient will require (preferably one), while at the same time maintaining low transvalvular gradients. In some implementations, the anterior-posterior distance at the top of the spacer or coaptation element is about 5 mm, and the medial-lateral distance of the spacer or coaptation element at its widest is about 10 mm. In some implementations, the overall geometry of the device 15000 can be based on these two dimensions and the overall shape strategy described above. It should be readily apparent that the use of other anterior-posterior distance anterior-posterior distance and medial-lateral distance as starting points for the device will result in a device having different dimensions. Further, using other dimensions and the shape strategy described above will also result in a device having different dimensions.

In some implementations, the outer paddles 15016 are jointably attached to the cap 15040 of the distal portion 15010 by connection portions 15080 and to the inner paddles 15018 by transition portions 15082. The inner paddles 15018 are jointably attached to the spacer or coaptation element by extensions 15084. In this manner, the anchors 15014 are configured similar to legs in that the inner paddles 15018 are like upper portions of the legs, the outer paddles 15016 are like lower portions of the legs, and the transition portions 15082 are like knee portions of the legs. As described above, the expandable coaptation element 10000 includes a nut 10090 and screw 10080 (see FIG. 31). Adjusting the nut 10090 adjusts the screw 10080, causing the expandable coaptation element 10000 to expand or contract.

In some implementations, the paddle frames 15020 are attached to the cap 15040 at the distal portion 15010 and extend to the transition portions 15082 between the inner and outer paddles 15018, 15016. In some implementations, the paddle frames 15020 are formed of a material that is more rigid and stiff than the material forming the paddles 15018, 15016 so that the paddle frames 15020 provide support for the paddles 15018, 15016.

The paddle frames 15020 can provide additional pinching force between the inner paddles 15018 and the expandable coaptation element 10000 and assist in wrapping the leaflets around the sides of the expandable coaptation element 10000. That is, the paddle frames 15020 can be configured with a round three-dimensional shape extending from the cap 15040 to the transition portions 15082 of the anchors 15014. The connections between the paddle frames 15020, the outer and inner paddles 15016, 15018, the cap 15040, and the expandable coaptation element 10000 can constrain each of these parts to the movements and positions of the treatment and/or repair devices described herein.

The wider configuration of the paddle frames 15020 can provide increased surface area compared to the inner paddles 15018 alone. The increased surface area can distribute the clamping force of the outer paddles 15016 and paddle frames 15020 against the native leaflets over a relatively larger surface of the native leaflets in order to further protect the native leaflet tissue.

In some implementations, the expandable coaptation element 10000 can include an expandable shell 15090. The shell 15090 can be configured as a solid surface, a lattice (e.g., a honeycomb or other pattern that leaves solid portions and cut out portions), a braided or woven material, or otherwise configured. The shell 15090 can be comprised of a single piece, two pieces, or a plurality of pieces. The shell 15090 can be composed of a rigid, semi-rigid, soft, or any other material suitable for implantation in the human body. One or more fasteners 15097 can be used to attach the shell 15090 to the expandable mechanism 10002.

In some implementations, a portion of the expandable mechanism 10002 that is covered by the shell can be selected such that the shell 15090 contacts the native valve leaflets and the expandable mechanism 10002 does not contact the native valve leaflets. For example, the expandable mechanism 10002 can be encapsulated by the shell 15090 or can be partially encapsulated by the shell 15090. In the illustrated example, the shell 15090 comprises two pieces that are opposite one another and move relative to one another. For example, one of the shell pieces can telescope into the other shell piece.

Referring now to FIGS. 34-35, examples of halves of the shell 15090 are shown. The shell 15090 can comprise two or more pieces and/or a flexible material, allowing the shell to conform to the overall size of the expandable mechanism 10002. Such a shell 15090 can provide a smooth contour for native valve leaflets to engage, either directly or through a cover.

A cover can be provided in a variety of different ways. For example, the components of the shell 15090 can be laminated with a polymer or covered with a fabric in order to improve the interaction with the leaflets and/or to seal the expandable mechanism 10002 from liquid. FIG. 34 illustrates a shell 15090 with a substantially solid surface. FIG. 35 illustrates a shell 15090 with cutouts that form a semisolid surface. In some implementations, the shell 15090 can be replaced by another structure, such as a balloon, balloon material, or other flexible material.

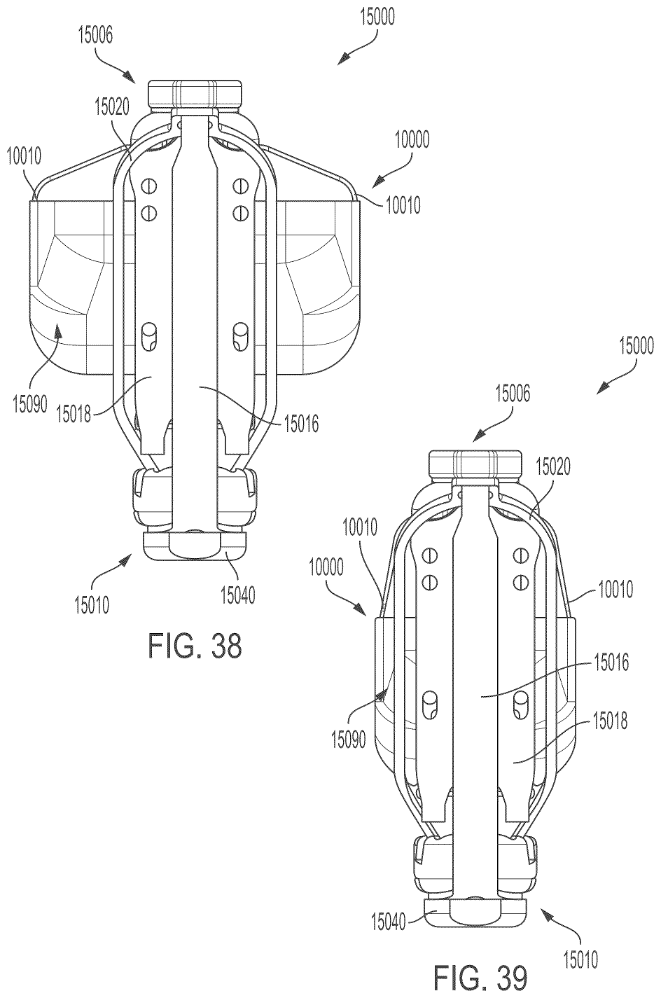

Referring now to FIG. 36, a front perspective view of the device 15000 described in FIG. 33 attached to native valve leaflets 20, 22 is shown. The expandable coaptation element 10000 is positioned between the leaflets 20, 22. A top plan view of the of the device 15000 is shown in FIG. 37 and side views of the device 15000 with the expandable coaptation element 10000 in an expanded configuration and a contracted configuration are shown in FIGS. 38 and 39 respectively. Adjusting the expandable mechanism 10002, for example by rotating the screw 10080, expands or contracts the shell 15090 to adjust flow through the native valve (reduce regurgitation through the native valve by increasing the size of the shell or increasing the flow through the native valve by reducing the size of the shell).

Referring to FIG. 38, the struts 10010 of the expandable mechanism 10002 are spread apart to move the halves of the shell 15090. As such, the shell 15090 has a wide configuration and takes up a larger space. Referring to FIG. 39, the struts 10010 of the expandable mechanism 10002 are close together to move or telescope the halves of the shell 15090 together. As such, the shell 15090 has a narrow configuration and takes up a smaller space. FIGS. 38 and 39 show a shell 15090 having two halves that are moved toward and away from one another by two struts 10010. However, in some implementations, the expandable coaptation element 10000 can have one, two, three, four or any number of struts that can be moved toward and away from a central axis of the expandable coaptation element 10000 and one, two, three, four, or any number of shell components that can be moved toward and away from the central axis of the expandable coaptation element 10000. The number of shell components can be the same or different than the number of moving struts.

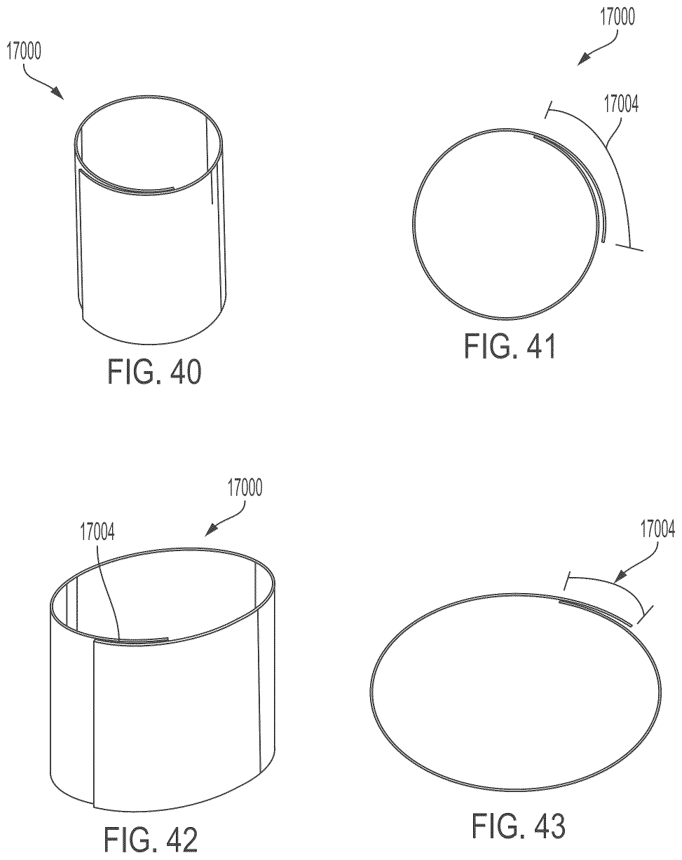

The expandable coaptation element 10000 can take a wide variety of different forms. For example, FIGS. 40-41 show an expandable sleeve 17000 that can be used instead of the shell 15090. The sleeve 17000 can be used with a wide variety of expandable mechanisms, such as the expandable mechanism 10002 described above or any other expandable mechanism described in this disclosure. The expandable mechanism can comprise one or more of a balloon, expandable container, expandable material, self-expanding material, self-expanding frame, stent, mechanically-expandable frame, pivoting and/or scissoring extensions and/or struts, expanding pully mechanism, Hoberman mechanism, cam, worm screw, rack and pinion, foam, etc.

FIGS. 40-41 show the expandable sleeve 17000 in various forms. The expandable sleeve 17000 can have a variety of different shapes. For example, the expandable sleeve can be cylindrical, conical, frustoconical, egg shaped, pyramid, a portion of a pyramid, diamond, combinations of these shapes, etc. Referring to FIGS. 40 and 41, in some implementations, the expandable sleeve 17000 has a circular shape with an overlap 17004. In one example, FIGS. 40 and 41 illustrate the sleeve 17000 in a contracted configuration.

FIGS. 42 and 43 illustrate the sleeve 17000 in an expanded configuration. For example, the expandable mechanism 10002 can be expanded to move the sleeve 17000 from the configuration shown in FIGS. 40 and 41 to the configuration shown in FIGS. 42 and 43. In FIGS. 42 and 43, the sleeve 17000 has the shape of an elliptical cylinder. FIGS. 42 and 43 illustrate only one of the many configurations that the expandable sleeve 17000 can take when expanded. The sleeve 17000 can be configured to expand to a wide variety of different shapes. For example, the sleeve can be configured to expand to a larger circular cylinder shape, a non-cylindrical shape, such as a shape that tapers from one end to the other end. As is illustrated by FIG. 42 the overlap 17004 decreases as the sleeve expands.

The sleeve 17000 can be made from a wide variety of different materials. For example, the expandable sleeve 17000 can be formed from a flexible material that can be a metal fabric, such as mesh, woven, braided, or formed in any other suitable way or a laser cut or otherwise cut flexible material. The material can be cloth, shape-memory alloy wire—such as Nitinol—to provide shape-setting capability, or any other flexible material suitable for implantation in the human body.

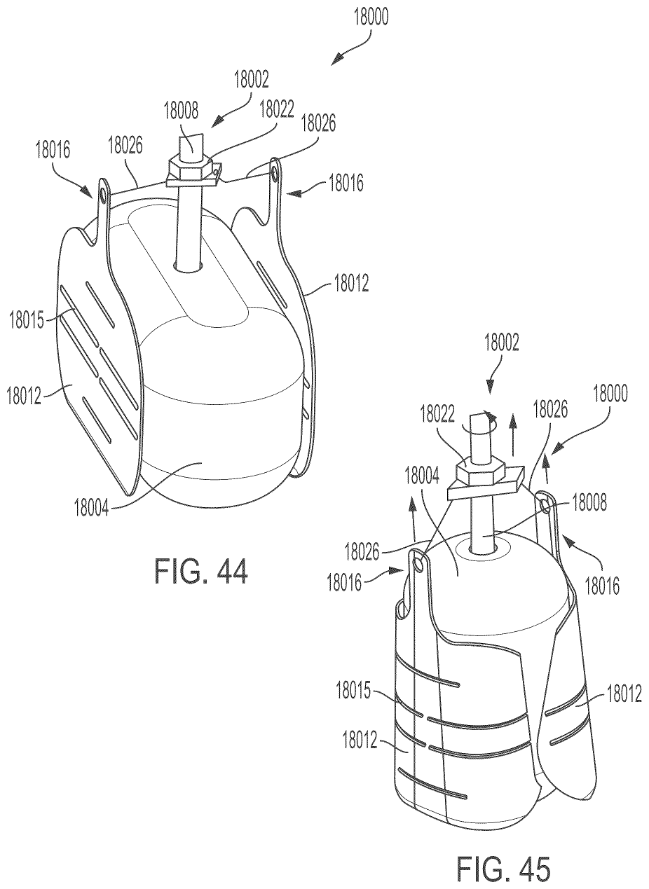

FIGS. 44-45 illustrate an example of an expandable coaptation element 18000. In the example illustrated by FIGS. 44 and 45, the expandable coaptation element 18000 can comprise one or more shape changing components 18012, an adjustment mechanism, and an optional fill material or core 18004. The shape changing component 18012 is configured to change size and/or shape when a force, such as a tensile force, is applied to the shape changing component. For example, the shape changing component 18012 can change from the flat or generally flat configuration shown in FIG. 44 to the curved configuration shown in FIG. 45 by applying a tensile force to the shape changing component 18012 with the adjustment mechanism 18002. In the example illustrated by FIGS. 44 and 45, the optional fill material or core 18004 is positioned between to shape changing components 18012. The optional fill material or core 18004 is compressed by the shape changing components 18012 as the shape changing components move from the configuration illustrated by FIG. 44 to the configuration illustrated by FIG. 45. In some implementations, the optional fill or core material 18004 keeps the shape changing components 18012 spaced apart to facilitate movement from the configuration illustrated FIG. 44 to the configuration illustrated by FIG. 45.

The shape changing components 18012 can take a wide variety of different forms having a variety of different expanded and contracted shapes. In the expanded condition illustrated by FIG. 44, the shape changing components 18012 can be generally parallel plate-like structures, as illustrated. In some implementations, the shape changing components 18012 can be cylindrical, conical, frustoconical, egg shaped, pyramid, a portion of a pyramid, diamond, combinations of these shapes, etc. in the expanded configuration. In the contracted condition illustrated by FIG. 45, the shape changing components 18012 can be generally rounded to compress the optional fill material or core 18004 to a generally cylindrical configuration, as illustrated. In some implementations, the shape changing components 18012 can be conical, frustoconical, egg shaped, pyramid, a portion of a pyramid, diamond, combinations of these shapes, etc. in the contracted configuration.

In some implementations, the shape changing components 18012 are formed from flat material and have cutouts 18015. The flat material and the cutouts 18015 are configured such that applying a force, such as a tensile force to the shape changing components 18012 causes the shape changing components two flex from a substantially flat configuration to a three-dimensional configuration. For example, kirigami techniques can be applied to the flat material to make the shape changing component 18012. The shape changing components 18012 can be made from a wide variety of different materials. For example, the shape changing components 18012 can be formed from any metal or polymeric material suitable for implantation in the human.

The shape changing components 18012 can be transformed from the expanded configuration to the compressed configuration in a wide variety of different ways. In the illustrated example, the shape changing components include a tab 18016. Applying tension to the tabs 18016 causes the shape changing components 18012 to transition from the configuration illustrated by FIG. 44 to the configuration illustrated by FIG. 45. However, the tabs 18016 are optional and other arrangements for applying force to the shape changing components 18012 can be used. As mentioned above, the shape changing components 18012 can be formed using kirigami, such that applying tension to the tabs 18016 causes the shape changing components to transform from a flat configuration to a curved, three-dimensional configuration.

A variety of different mechanisms can be used to move the shape changing components 18012 from the expanded configuration to the contracted configuration. In some implementations, a screw or bolt 18008 and a nut 18022 or other fastening arrangement can be used to move the shape changing components 18012 from the expanded configuration to the contracted configuration. Rotating the nut 18022 relative to the screw or bolt 18008 can apply tension to the shape changing components 18012 to move the shape changing components from the expanded configuration (FIG. 44) to the contracted configuration (FIG. 45). For example, a line 18026 can be connected to the tab 18016 and be coupled to the nut 18022. Rotating the nut 18022 pulls the line 18026 to move the shape changing components 18012 from the expanded configuration to the contracted configuration. In some implementations, tension on the tabs 18016 causes the shape changing components 18012 to transition from the configuration illustrated by FIG. 44 to the configuration illustrated by FIG. 45.

The optional fill material or core 18004 can take a variety of different forms. The optional fill material or core 18004 can be composed of a soft, semi-soft, semi-hard. The optional fill material or core 18004 can be hollow or solid. The optional fill material or core 18004 can be sponge-like and porous, can be composed of a material such as mesh, woven, braided, or formed in any other suitable way or a laser cut or otherwise cut flexible material. The material can be cloth, shape-memory alloy, foam, sponge-such as Poron or any other flexible material suitable for implantation in the human body. Applying force to the shape changing components 18012 causes the shape changing components to change shape and, therefore, reshape the optional fill material or core 18004.

In the illustrated example, when the shape changing components 18012 are in the non-tensioned configuration of FIG. 44, the optional fill material or core 18004 can have an oblong shape. When the shape changing components 18012 are in the tensioned configuration of FIG. 45, the optional fill material or core 18004 can have a cylindrical or generally cylindrical configuration. However, the optional fill material or core 18004 can have a variety of different shapes in the expanded and compressed configurations. For example, the optional fill material can be cylindrical, conical, frustoconical, egg shaped, pyramid, a portion of a pyramid, diamond, wedge shaped, combinations of these shapes, etc. in the expanded can compressed configurations.

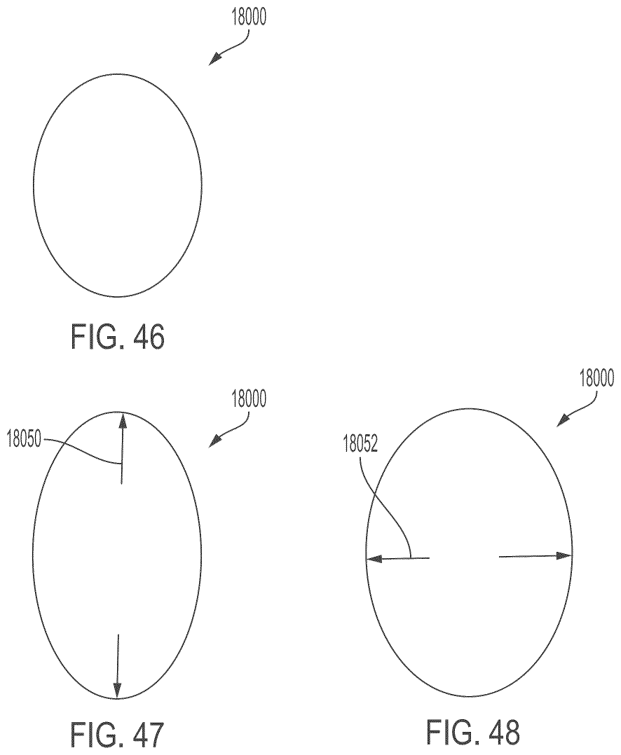

The expandable coaptation elements 18000, as well as the other expandable coaptation elements disclosed herein (10000, 17000, etc.), can be configured to expand and contract in a variety of different ways. FIGS. 46-48 are schematic top plan views of the expandable coaptation element. FIG. 46 illustrates the expandable coaptation element 18000 in a normal or starting position. The expandable coaptation element 18000 can expand and/or contract from the configuration illustrated by FIG. 46 in a variety of different ways. For example, in some implementations, the expandable coaptation element 18000 can:
Expand or contract only in the direction 18050 illustrated by FIG. 47;
Expand or contract only in the direction 18052 illustrated by FIG. 48;
Simultaneously expand and contract in the direction 18050 illustrated by FIG. 47 and the direction 18052 illustrated by FIG. 48;
Sequentially expand or contract such that the expandable coaptation element 18000 expands or contracts first in the direction 18050 illustrated by FIG. 47 and then expands or contracts in the direction 18052 illustrated by FIG. 48;
Sequentially expand or contract such that the expandable coaptation element 18000 expands or contracts first in the direction 18052 illustrated by FIG. 48 and then expands or contracts in the direction 18050 illustrated by FIG. 47;
Independently expand or contract in the direction 18050 illustrated by FIG. 47 and in the second direction 18052 illustrated by FIG. 48;
Partially expand or contract in the direction 18050 illustrated by FIG. 47 and then expand or contract in both the direction 18050 illustrated by FIG. 47 and the direction 18052 illustrated by FIG. 48; and/or
Partially expand or contract in the direction 18052 illustrated by FIG. 48 and then expand or contract in both the direction 18050 illustrated by FIG. 47 and the direction 18052 illustrated by FIG. 48.

As such, shape changing components 18012 and/or the expandable material 18004 can expand sequentially, simultaneously, or otherwise when the shape changing component 18012 is manipulated between the tensioned and non-tensioned configuration.

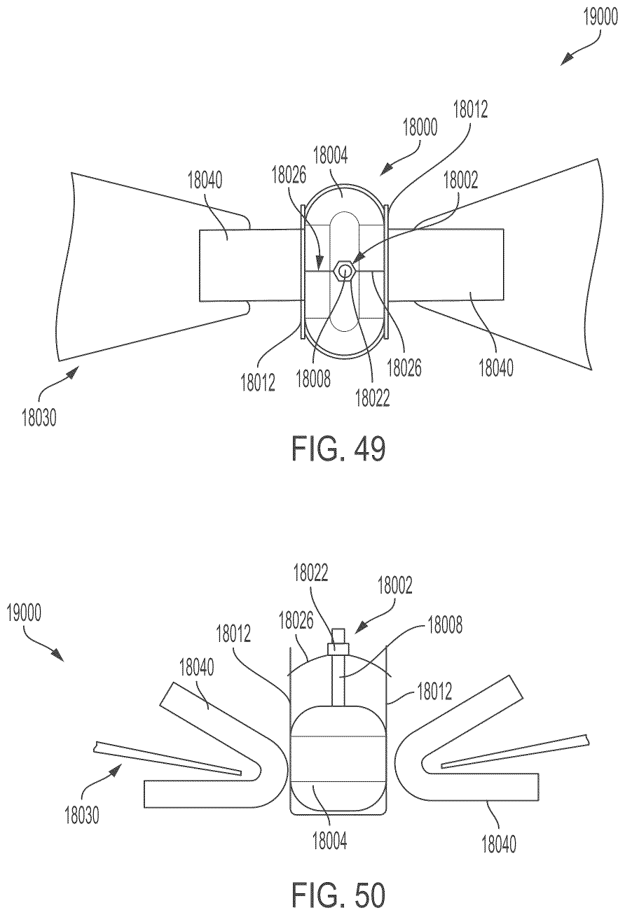

FIGS. 49-52 Illustrate an example of a device 19000 (e.g., valve repair device, valve treatment device, implantable device, implant, etc.) that includes the expandable coaptation element 18000 illustrated by FIGS. 44 and 45. The expandable coaptation element 18000 can be used in a variety of different devices, including but not limited to, any of the treatment and/or repair devices disclosed in the present application. For example, the expandable coaptation element 18000 can be used in the device that is schematically illustrated in FIGS. 8-14. The implantable device 19000 is one of the many different configurations that the device that is schematically illustrated in FIGS. 8-14 with an expandable coaptation element 18000 can take.

The device 19000 can include any other features for a device (e.g., a treatment and/or repair device, etc.) discussed in the present application, and the expandable coaptation element 18000 can be positioned to engage leaflets 30, 32, 34 (see FIGS. 7 and 34) or leaflets 20, 22 (see FIGS. 6 and 36) as part of any suitable device 19000 (e.g., any treatment and/or repair device disclosed in the present application). The device 19000 can be deployed from a delivery sheath, a device catheter, and/or an implant catheter. The device 19000 can include an adjustment mechanism 18002 and an anchor portion having two or more anchors, such as the anchor portion and anchors described herein. In the example illustrated by FIGS. 49-52, the anchors of the device 19000 comprises clasps 18040.

In the example illustrated by FIGS. 49-52, the clasps 18040 attach the adjustable coaptation element 18000 to the leaflets of a native valve 18030 (e.g., native mitral valve or native tricuspid valve). The clasps 18040 position the adjustable coaptation element between the leaflets of the native valve 18030. In some implementations, the leaflet clasp or plurality of leaflet clasps 18040 attach to the top or atrial side and bottom or ventricular side of a leaflet of a native valve 18030.

The adjustable coaptation element is 18000 adjusted to change the shape and/or size of the area of the native valve that is blocked by the adjustable coaptation element 18000. FIGS. 49 and 50 illustrate the device 19000 attached to the native valve 18030 with the adjustable coaptation element 18000 in an expanded configuration. Relative rotation of the screw or bolt 18008 and the nut 18022 can apply tension to the shape changing components 18012. For example, a line 18026 can be connected to the tab 18016 and be coupled to the nut 18022. Rotating bolt 18008 relative to the nut 18022 pulls the line 18026 to move the shape changing components 18012 from the expanded configuration of FIG. 44 to the contracted configuration of FIG. 45.

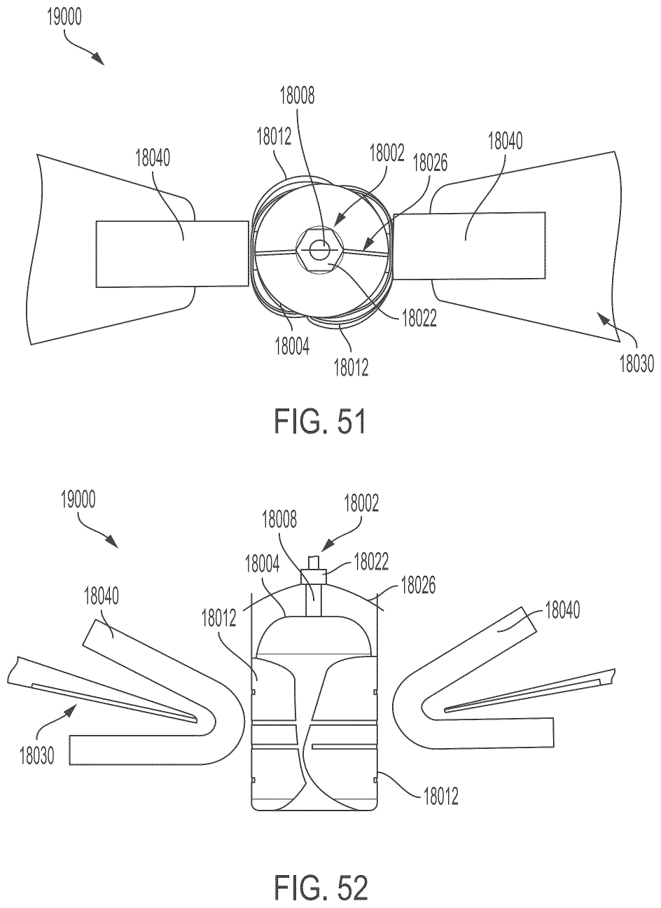

FIG. 49 shows a top plan view of the adjustable coaptation element 18000 in an expanded configuration placed between leaflets of the native valve 18030. The screw or bolt 18008 can extend into or through the spacing material 18004. FIG. 50 shows side perspective view of the expanded adjustable coaptation element 18000 between leaflets of the native valve 18030. FIG. 51 shows a top plan view of the adjustable coaptation element 18000 in a compressed configuration placed between leaflets of the native valve 18030. FIG. 52 shows side perspective view of the compressed adjustable coaptation element 18000 between leaflets of the native valve 18030.

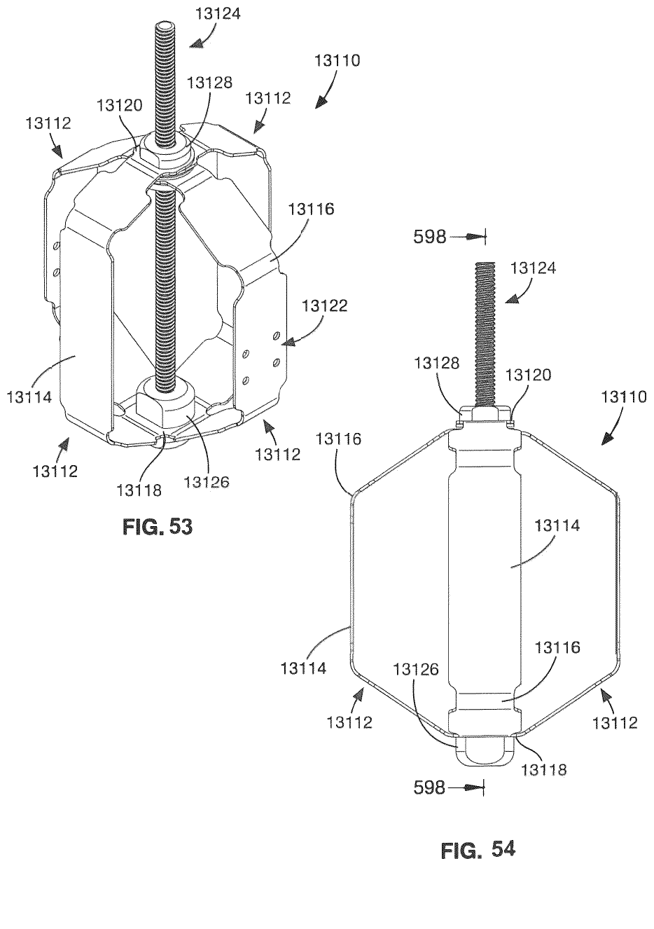

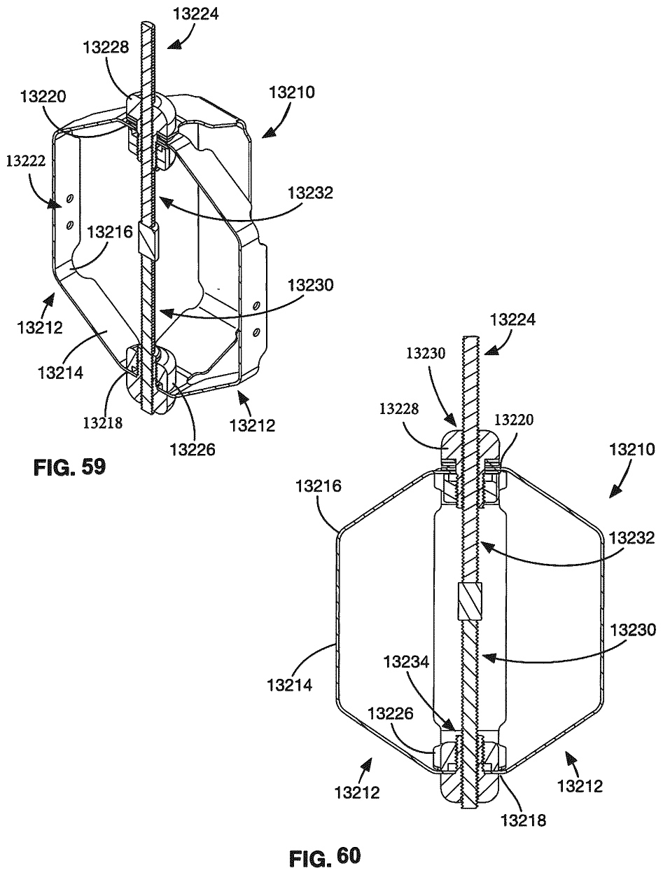

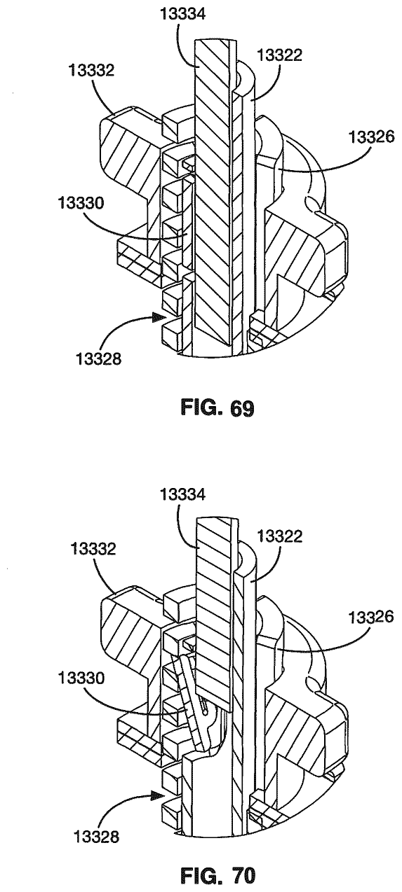

A variety of different mechanisms can be used to move the expandable or adjustable coaptation elements between expanded and contracted configurations. FIGS. 53-70 illustrate examples of expandable mechanisms that can be used with any of the devices herein. In the example illustrated by FIGS. 53-56, the expandable mechanism expands and contracts in two directions. A device (e.g., a treatment device, a repair device, an implantable device, etc.) that includes the expandable mechanism can include any other features for a device discussed in the present application, and the device can be positioned to engage valve tissue as part of any suitable treatment and/or repair system (e.g., any treatment and/or repair system disclosed in the present application). The device can be deployed from a delivery sheath or means for delivery by a pusher, such as a rod or tube as described above.

The device can include a coaptation portion and an anchor portion having two or more anchors, such as the anchor portions and anchors described herein. The coaptation portion includes an expandable mechanism 13110 that can be actuated between a contracted condition and an expanded condition. An optional outer surface, such as the shell 15090, sleeve 17000, shape changing component 18012, etc. can be included to cover a portion or all of the expandable mechanism 13110.

The expandable mechanism 13110 is formed from one or more struts 13112. The struts 13112 have a plurality of rigid portions 13114 that are connected together by hinge portions 13116 that enable the rigid portions 13114 to bend relative to each other. A fixed end portion 13118 is connected to each of the struts 13112 by the hinge portions 13116. The struts 13112 extend from the fixed end portion 13118 to a moveable end portion 13120 connected to each of the struts 13112 by one of the hinge portions 13116. One or more of the rigid portions 13114 can include an attachment region 13122 for attaching another component of the device.

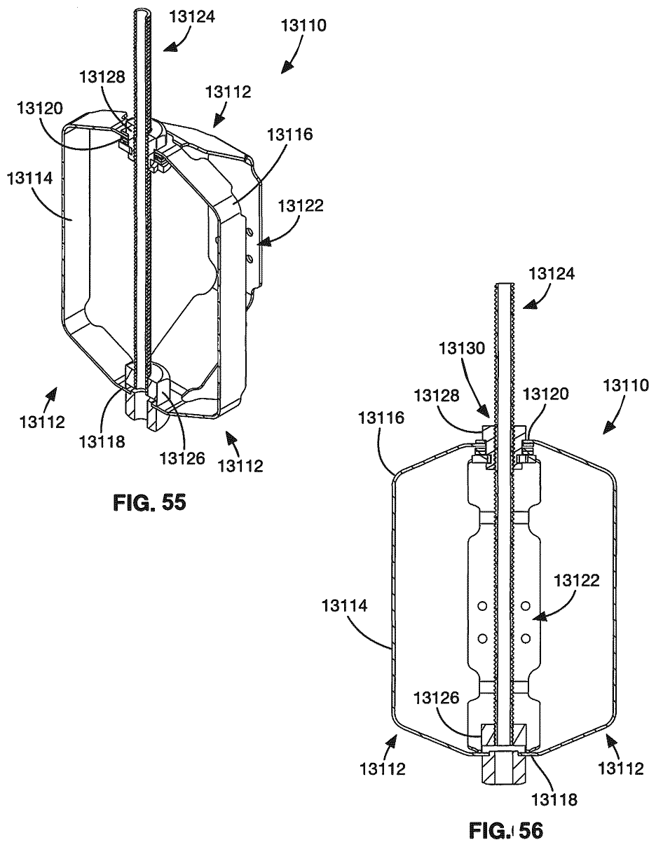

The struts 13112 are moved between a collapsed condition and an expanded condition by the actuation of a threaded actuation shaft 13124 that extends through the expandable mechanism 13110 to a fixed actuation member 13126. The fixed end portion 13118 is attached to the fixed actuation member 13126 and the moveable end portion 13120 are attached to the moveable actuation member 13128. The fixed actuation member 13126 extends proximally from the fixed actuation member 13126 and through a moveable actuation member 13128. The threaded actuation shaft 13124 can be hollow and the fixed actuation member 13126 and the moveable actuation member 13128, as shown in FIGS. 55-56, so that other actuation members and other components of the device can extend through the expandable mechanism 13110 and can be moved or actuated independently of the actuation of the expandable mechanism 13110.

The moveable actuation member 13128 includes a threaded opening 13130 that engages the threads of the threaded actuation shaft 13124 so that rotation of the threaded actuation shaft 13124 relative to the moveable actuation member 13128—or vice versa—moves the moveable actuation member 13128 toward and away from the fixed actuation member 13126. That is, rotating the moveable actuation member 13128 via an actuation shaft or other mechanism (not shown) causes the moveable actuation member 13128 to move proximally away from the fixed actuation member 13126 and distally toward the fixed actuation member 13126. As the moveable actuation member 13128 is moved along the threaded actuation shaft 13124, the struts 13112 expand and contract to change the overall width of the expandable mechanism 13110. The expandable mechanism 13110 has a minimum width when the struts 13112 are stretched to an almost nearly or straight condition and/or when the fixed end portion 13118 and the moveable end portion 13120 are moved close to each other so that the rigid portions 13114 are folded against each other. In this way, the expandable mechanism 13110 can be caused to expand and contract laterally to accommodate different size and shape gaps 26 (see FIG. 6) left between the leaflets 20, 22 during diastole when the native heart valve closes around the device.

The rigid portions 13114 of the struts 13112 and also an optional cover covering the struts 13112 can include shaped portions or can include flexible or pliable portions to allow the surface of the device to conform to the shape of the native leaflets 20, 22 when the leaflets 20, 22 close against the device to improve engagement between the struts 13112 and the native leaflets 20, 22. While four struts 13112 are shown, any number of struts 13112 can be combined to provide expanding and contracting coaptation surfaces. Different struts can include rigid portions 13114 having different shapes and lengths to provide differing rates of expansion and contraction and different maximum expansion positions to form a wide variety of differently shaped expandable mechanisms 13110.

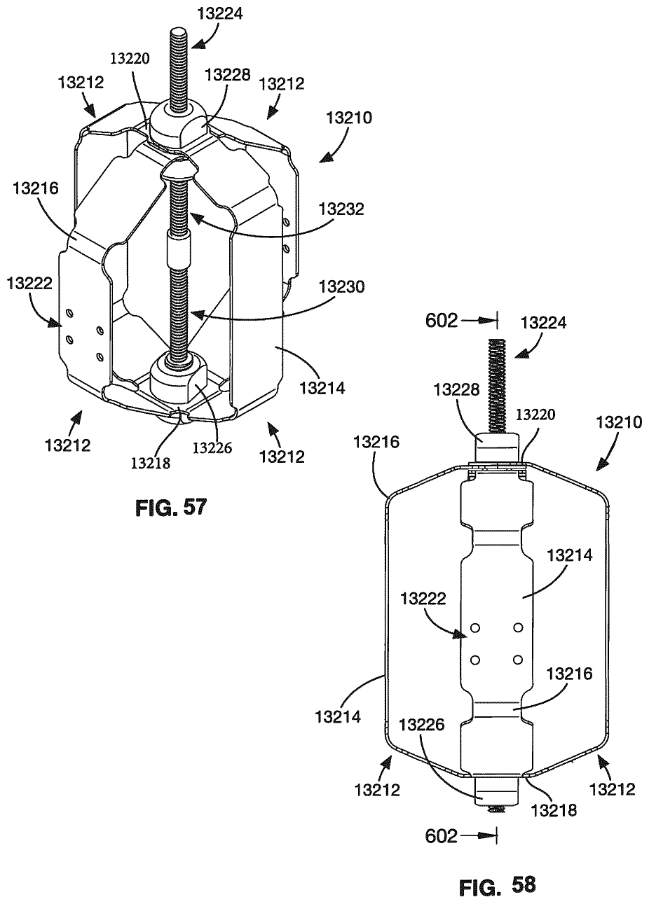

Referring now to FIGS. 57-60, an example of an expandable mechanism 13210 for use in a device (e.g., a treatment device, a repair device, an implantable device, an implant, etc.) is shown. The device can include any other features for a device (e.g., treatment device, repair device, etc.) discussed in the present application, and the device can be positioned to engage valve tissue as part of any suitable treatment and/or repair system (e.g., any treatment system and/or repair system disclosed in the present application).

The device can be deployed from a delivery sheath or means for delivery by a pusher, such as a rod or tube as described above. The device can include a coaptation portion and an anchor portion having two or more anchors, such as the anchor portion and anchors described herein. In some implementations, the coaptation portion includes an expandable mechanism 13210 that can be actuated between a contracted condition and an expanded condition. In some implementations, an optional outer surface, such as the shell 15090, sleeve 17000, shape changing component 18012, etc. can be included to cover a portion or all of the expandable mechanism 13210.

In some implementations, the expandable mechanism 13210 is formed from one or more struts 13212. In some implementations, the struts 13212 have a plurality of rigid portions 13214 that are connected together by hinge portions 13216 that enable the rigid portions 13214 to pivot relative to each other. In some implementations, a first end portion 13218 is connected to each of the struts 13212 by the hinge portions 13216. In some implementations, the struts 13212 extend from the first end portion 13218 to a second end portion 13220 connected to each strut 13212 by one of the hinge portions 13216. One or more of the rigid portions 13214 can include an attachment region 13222 for attaching another component of the device.

In some implementations, the struts 13212 are moved between a collapsed condition and an expanded condition by the actuation of a threaded actuation shaft 13224 that extends through a first actuation member 13226 and a second actuation member 13228 of the expandable mechanism 13210. In some implementations, the first end portion 13218 of the struts 13212 is attached to the first actuation member and the second end portions 13220 are attached to the second actuation member 13228.

In some implementations, the threaded actuation shaft 13224, the first actuation member 13226, and the second actuation member 13228 can be hollow so that other actuation members and other components of the device can extend through the expandable mechanism 13210 and can be moved or actuated independently of the actuation of the expandable mechanism 13210.

In some implementations, the threaded actuation shaft 13224 includes a first threaded portion 13230 extending from a distal end to a second threaded portion 13232 and the second threaded portion 13232 extends from the first threaded portion 13230 to a proximal end. In some implementations, the threads of the first threaded portion 13230 and the second threaded portion 13232 are oriented in opposite directions, i.e., if the threads of the first threaded portion 13230 are right-handed threads, then the threads of the second threaded portion 13232 are left-handed threads, and vice versa.

In some implementations, the first actuation member 13226 engages the first threaded portion 13230 and includes a threaded opening 13234 that has threads that match the orientation of the threads of the first threaded portion 13230. In some implementations, the second actuation member 13228 engages the second threaded portion 13232 and includes a threaded opening 13234 that has threads that match the orientation of the threads of the second threaded portion 13232. Consequently, rotating the threaded actuation shaft 13224 in one direction moves the first actuation member 13226 and the second actuation member 13228 in opposite directions. Because the first actuation member 13226 and the second actuation member 13228 move in opposite directions at the same rate when the threaded actuation shaft 13224 is rotated, a midplane formed between the first actuation member 13226 and the second actuation member 13228 does not move as the expandable mechanism 13210 is expanded and contracted.

In some implementations, during operation of the expandable mechanism 13210, for example, rotating the threaded actuation shaft 13224 can cause the first actuation member 13226 to move in a proximal direction and the second actuation member 13228 to move in a distal direction, thereby decreasing the distance between the first actuation member 13226 and the second actuation member 13228. In some implementations, rotating the threaded actuation shaft 13224 in the opposite direction would cause the first actuation member 13226 to move in a distal direction and the second actuation member 13228 to move in a proximal direction, thereby increasing the distance between the first actuation member 13226 and the second actuation member 13228. In this way, rotation of the threaded actuation shaft 13224 is used to expand and contract the struts 13212 of the expandable mechanism 13210. In this way, rotation of the threaded actuation shaft 13224 causes the outer perimeter or size of the expandable mechanism 13210 to increase and decrease to accommodate different size and shape gaps 26 (see FIG. 6) left between the leaflets 20, 22 during diastole when the native heart valve closes around the device.

In some implementations, the rigid portions 13214 of the struts 13212 and/or a shell, sleeve, etc. covering the struts 13212 can include shaped portions or can include flexible or pliable portions to allow the surface of the device to conform to the shape of the native leaflets 20, 22 when the leaflets 20, 22 close against the device to improve engagement between the struts 13212 and the native leaflets 20, 22. While four struts 13212 are shown, any number of struts 13212 can be combined to provided expanding and contracting coaptation surfaces. Different struts can include rigid portions 13214 having different shapes and lengths to provide differing rates of expansion and contraction and different maximum expansion positions to form a wide variety of differently shaped expandable mechanisms 13210.

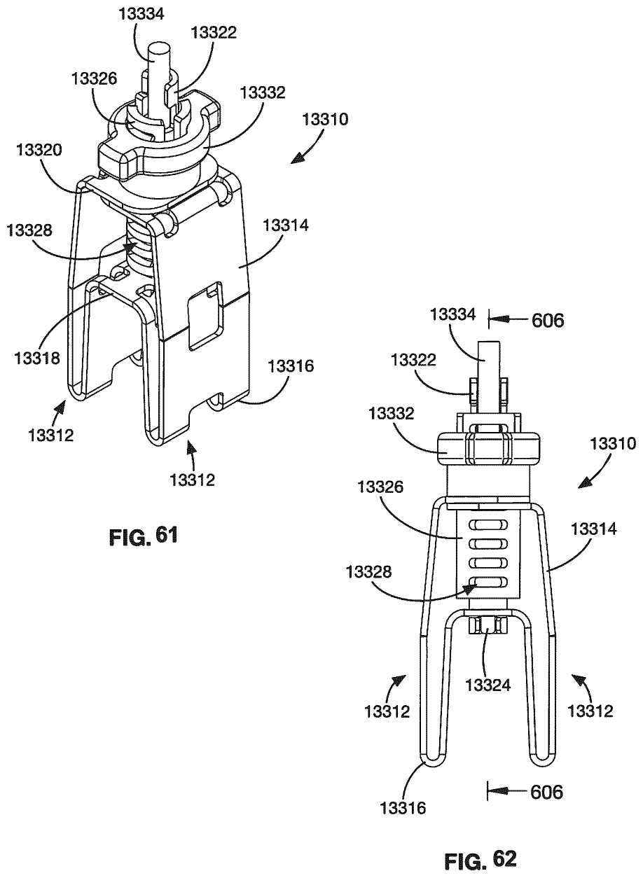

Referring now to FIGS. 61-70, an expandable mechanism 13310 for use in a device (e.g., a treatment device, a repair device, an implantable device, an implant, etc.) is shown. The device can include any other features for a device (e.g., treatment device, repair device, etc.) discussed in the present application, and the device can be positioned to engage valve tissue as part of any suitable treatment and/or repair system (e.g., any treatment and/or repair system disclosed in the present application). The device can be deployed from a delivery sheath or means for delivery by a pusher, such as a rod or tube as described above.

In some implementations, the device can include a coaptation portion and an anchor portion having two or more anchors, such as the anchor portion and anchors described herein. In some implementations, the coaptation portion 13304 includes an expandable mechanism 13310 that can be actuated between a contracted condition and an expanded condition. An optional outer surface, such as the shell 15090, sleeve 17000, shape changing component 18012, etc. can be included to cover a portion or all of the expandable mechanism 13310.

The expandable mechanism 13310 is formed from one or more struts 13312. The struts 13312 have a plurality of rigid portions 13314 that are connected together by hinge portions 13316 that enable the rigid portions 13314 to pivot relative to each other. A first end portion 13318 is connected to each of the struts 13312 by the hinge portions 13316. The struts 13312 extend from the first end portion 13318 to a second end portion 13320 connected to each strut 13312 by one of the hinge portions 13316. One or more of the rigid portions 13314 can include an attachment region for attaching another component of the expandable mechanism 13310 or of the repair device.

The expandable mechanisms disclosed herein (and useable with any of the devices herein) can be moved to various expanded and contracted positions and held in place by a variety of different mechanisms. As is disclosed above, threaded members can be used to move the expandable mechanisms to various expanded and contracted positions and hold the expandable mechanisms in place. Additionally or alternatively, the expandable mechanisms herein can comprise one or more of a balloon, expandable container, expandable material, self-expanding material, self-expanding frame, stent, mechanically-expandable frame, pivoting and/or scissoring extensions and/or struts, expanding pully mechanism, Hoberman mechanism, cam, worm screw, rack and pinion, foam, etc.

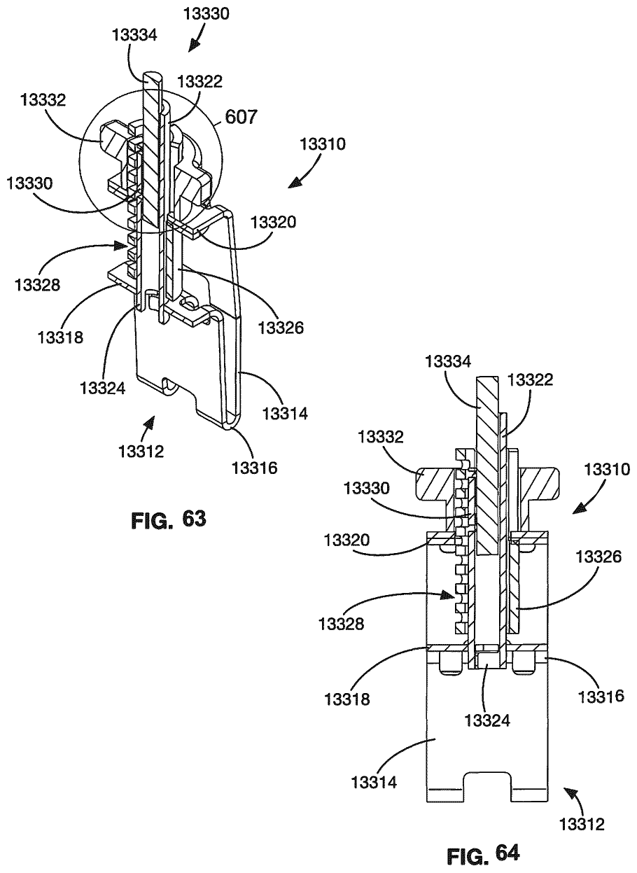

A wide variety of other mechanisms can additionally or alternatively be used. For example, FIGS. 63-64 and 67-70 illustrate an example of a mechanism for moving the expandable mechanisms to various expanded and contracted positions and holding the expandable mechanisms in place. In this example, the struts 13312 are moved between a collapsed condition and an expanded condition by the extension and retraction of an actuation tube 13322. The actuation tube 13322 extends to a distal end 13324 that is attached to the first end portion 13318 and moves within a latch tube 13326 that includes a plurality of openings 13328 for engaging with a latch member 13330 of the actuation tube 13322. A retention or securing member 13334 secures the connection between the actuation tube 13322 and latch tube 13326 by engaging the latch member 13330; the relationship between the actuation tube 13322, the latch tube 13326, the latch member 13330, and the securing member 13334 is shown in FIGS. 63-64 and 67-70 and is described below.

In some implementations, the latch tube 13326 is attached to a proximal collar or head 13332 of the device that can be coupled to a delivery mechanism for delivering the device within the native valve. In some implementations, the proximal collar or head 13332 is also attached to the second end portions 13320 of the struts 13312. Consequently, extension and retraction of the actuation tube 13322 changes the distance between the first end portion 13318 and the second end portion 13320 of the struts 13312 to cause the struts 13312 to move between the contracted and expanded conditions. The proximal collar or head 13332 can be engaged and actuated in a wide variety of ways described herein to facilitate the deployment and/or implantation of the devices described herein, such as, for example, by opening and closing paddles of the device.

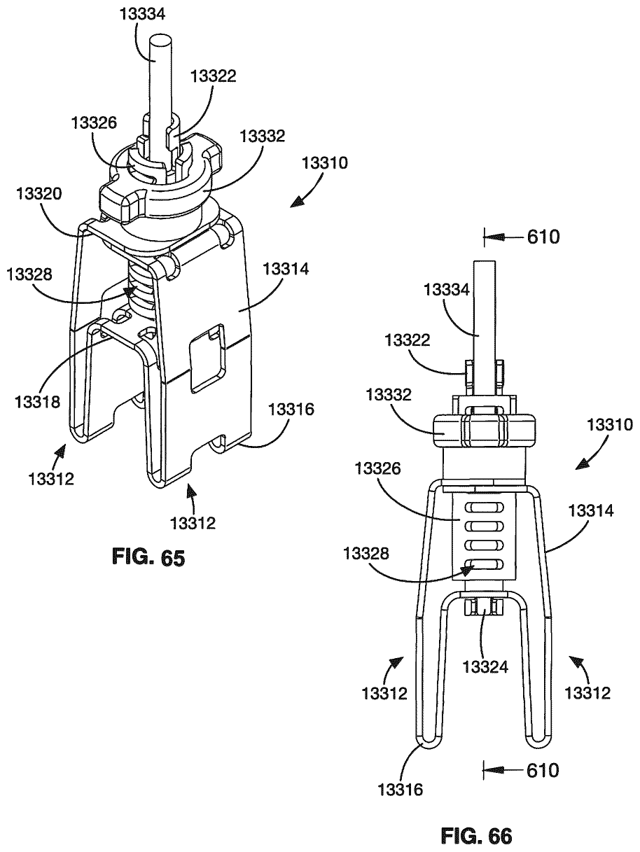

In some implementations, the latch member 13330 of the actuation tube 13322 can be moved between a latched and an unlatched condition. The actuation tube 13322 can include any number of latch members 13330 suitable for engaging corresponding openings 13328 in the latch tube 13326. Referring now to FIGS. 61-64 and 69, the latch member 13330 of the actuation tube 13322 is shown in an unlatched condition. When the latch member 13330 is in the unlatched condition, the actuation tube 13322 can be moved freely along the length of the latch tube 13326 to extend and retract the first end portion 13318, thereby expanding and contracting the expandable mechanism 13310. In the latched condition, shown in FIGS. 65-68 and 70, the latch member 13330 in the latched condition engages the openings 13328 of the latch tube 13326 to secure the position of the actuation tube 13322 relative to the latch tube 13326.

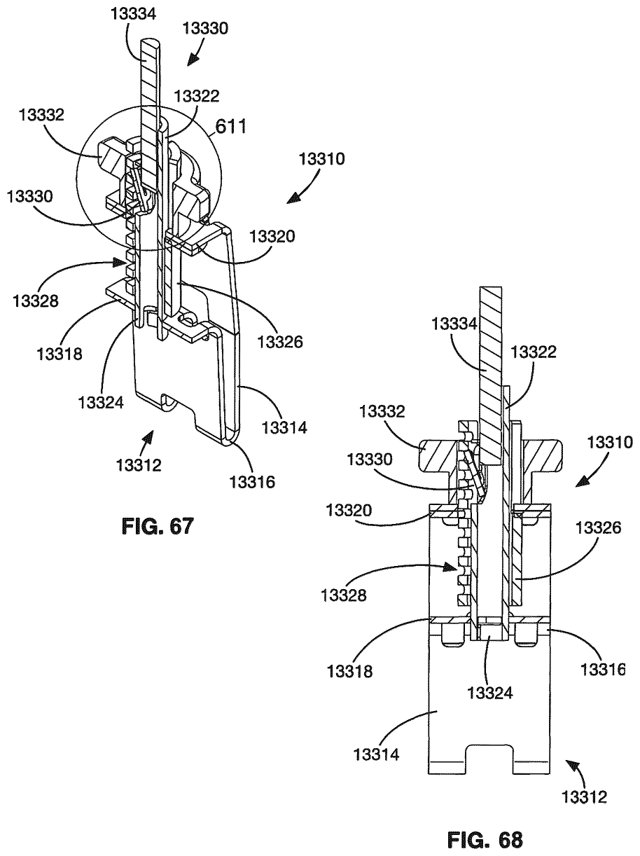

In some implementations, the latch member 13330 can optionally be integrally formed in the side of the actuation tube 13322 by laser-cutting a portion of the actuation tube 13322. The latch member 13330 is bent and shape-set in the latched condition so that in a free state the latch member 13330 is angled relative to a central axis of the actuation tube 13322. For example, as shown in FIGS. 67-68, the proximal or upper end of the latch member 13330 is biased radially outward and the opposite lower or distal end of the latch member 13330 is biased radially inward.

In some implementations, the proximal end of the latch member 13330 can be biased radially inward and the opposite distal end of the latch member 13330 can be biased radially outward. In some implementations, the expandable mechanism 13310 is biased to expand or contract and the latch member 13330 is angled outward to oppose the bias of the expandable mechanism 13310. In an example where the expandable mechanism 13310 is biased to cause the actuation tube 13322 to move in the proximal direction, the proximal end of the latch member 13330 can be biased radially outward so that the latch member 13330 engages the openings 13328 of the latch tube 13326 such that the proximal movement of the actuation tube 13322 is resisted by the latch member 13330.

In some implementations, the latch member 13330 is retained in the unlatched condition by the retaining or securing member 13334 that prohibits the latch member 13330 from pivoting into the latched condition. During deployment and/or implantation of the device the width of the expandable mechanism 13310 is adjusted by extending and retracting the actuation tube 13322. When the desired width of the expandable mechanism has been reached, the retention securing member 13334 is retracted from the actuation tube 13322 to allow the latch member 13330 to pivot into the latched condition to engage the openings 13328 of the latch tube 13326. In some implementations, the extension and retraction of the actuation tube 13322 causes size of the expandable mechanism 13310 to increase and decrease to accommodate different size and shape gaps 26 (see FIG. 6) left between the leaflets 20, 22 during diastole when the native heart valve closes around the coaptation element. Locking the actuation tube 13322 in place relative to the latch tube 13326 via the latch member 13330 maintains the width of the expandable mechanism 13310 against the force applied by the leaflets 20, 22.

In some implementations, the retention or securing member 13334 can optionally have a tapered distal end to facilitate re-engagement of the latch member 13330 to cause the latch member 13330 to pivot from the latched condition to the unlatched condition, thereby enabling further adjustment of the width of the expandable mechanism 13310.

In some implementations, the rigid portions 13314 of the struts 13312 and/or a shell, sleeve, etc. covering the struts 13312 can include shaped portions or can include flexible or pliable portions to allow the surface of the expandable coaptation element to conform to the shape of the native leaflets 20, 22 when the leaflets 20, 22 close against the implanted device to improve engagement between the struts 13312 and the native leaflets 20, 22. While four struts 13312 are shown, any number of struts 13312 can be combined to provided expanding and contracting coaptation surfaces. Different struts can include rigid portions 13314 having different shapes and lengths to provide differing rates of expansion and contraction and different maximum expansion positions to form a wide variety of differently shaped expandable coaptation mechanisms 13310.

Expandable coaptation elements can take a variety of different forms. In some implementations, the expandable coaptation element is made from a lattice of struts and/or can have a stent-like configuration. The lattice of struts can be formed in a variety of different ways. For example, the expandable coaptation element can be cut (e.g., laser cut) from sheet material, molded, made by additive manufacturing techniques (e.g., 3D printing), etc.

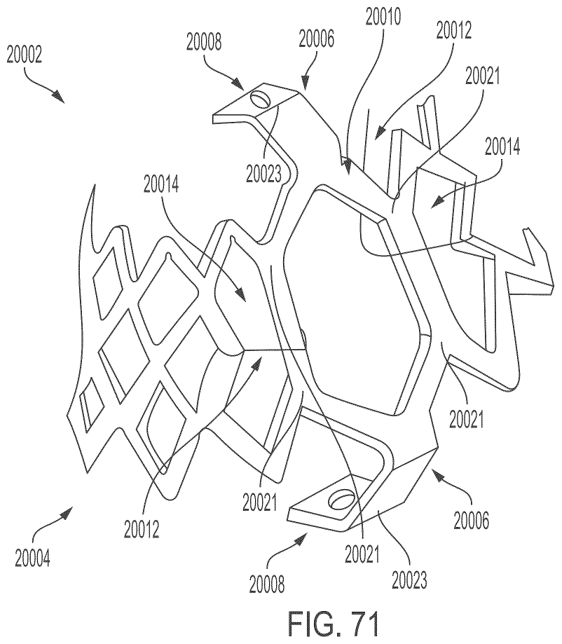

Referring now to FIGS. 71-74, example portions of an expandable coaptation element 20002 for use in a device (e.g., a treatment device, a repair device, an implantable device, an implant, etc.) is shown. The expandable coaptation element 20002 can be used in a variety of different devices, including but not limited to, any of the treatment and/or repair devices disclosed in the present application. For example, the expandable coaptation element 20002 can be used in the device that is schematically illustrated in FIGS. 8-14. A device having the expandable coaptation element 20002 can include any other features for a device (e.g., treatment device, repair device, etc.) discussed in the present application and can be positioned to engage valve tissues as part of any suitable treatment and/or repair system (e.g., any treatment and/or repair system disclosed in the present application). The expandable coaptation element 20002 can be deployed from a delivery sheath or means for delivery by a pusher, such as a rod or tube.

Referring to FIGS. 71-73, the coaptation element 20002 can comprise a cellular frame 20004 that can allow for control of both width and length of the coaptation element to adapt to differently sized leaflet gaps. Width and length are both relative terms, defined by the plane of the native valve. In one example, width is the dimension on an axis between the leaflets, while length is transverse to the width (e.g., between commissures). The expandable coaptation element 20002 can be composed of a shape-memory alloy-such as Nitinol—to provide shape-setting capability, or any other flexible material suitable for implantation in the human body.

The expandable coaptation element 20002 can be cellular, tubular, elliptical/bowl shaped, or any other shape conducive to implantation between leaflets of a native valve. In some implementations, the expandable coaptation element 20002 comprises a stent-like cellular frame with triangular, diamond, and/or hexagonal honeycomb cells. In some implementations, the structure can be tubular wherein the length is greater than the width with the length-ends having an arc shape. The coaptation element 20002 can have a plurality of ends (on the length of the coaptation element) and/or a plurality of sides (on the width of the coaptation element).

In some implementations, the coaptation element 20002 can have one or a plurality of tabs or extensions 20006 on the side or plurality of sides (e.g., at each point on the frame 20004 where the minor axis of the cross-section intersects the frame). The coaptation element 20002 can be expanded and/or contracted vertically and/or horizontally. Expansion and/or contraction can be achieved through actuation portions or actuation members 20008 that extend from the tabs or extensions 20006. The actuation portions or actuation members 20008 can be coupled to the frame 20004, such as being integrally formed with the frame as illustrated. In some implementations, the actuation portions or actuation members 20008 can be diametrically disposed around a central axis of the expandable coaptation element 20002. The side or plurality of sides of the expandable coaptation element 20002 referenced in FIGS. 71-74 can expand sequentially, simultaneously, or otherwise when being manipulated and/or actuated.

The expandable coaptation element 20002 can expand and/or contract in a variety of different ways. For example, the expandable coaptation element 20002 can:
Expand or contract only in length;
Expand or contract only in width;
Simultaneously expand and contract in length and width;
Sequentially expand or contract such that the expandable coaptation element 20002 expands or contracts first in length and then expands or contracts in width;
Sequentially expand or contract such that the expandable coaptation element 20002 expands or contracts first in width and then expands or contracts in length;
Independently expand or contract in length and width;
Partially expand or contract in length and then expand or contract in both length and width; and/or
Partially expand or contract in width and then expand or contract in both length and width.

As such, shape changing elements 20002 can expand sequentially, simultaneously, or otherwise.

In some implementations, one or more secondary controls 20012 can be used to change the size of one or more cells independently of the actuation portions or members 20008. The secondary control 20012 can take a wide variety of different forms. For example, the secondary control can comprise a line, such as a suture, a wire, etc., an adjustable width fastener, a spring, or any other component capable of being controlled to change the size of one or more of the cells 20014. The one or more secondary controls 20012 can be attached to the individual cells 20014 of the frame 20004. The individual cells 20014 can be specialized, such as by having a different shape (e.g., hexagonal, heptagonal, rectangular, triangular, etc.), different strut thickness, different strut width, etc. than other cells of the frame 20004. In some implementations, the secondary control 20012 can be used to adjust the width of the frame 20004 by pulling on the specialized cell to narrow the specialized cell 20014 and, as a result, the frame 20004 as a whole.

The narrowing and expanding of the individual cells 20014 with the secondary control 20012 changes the shape of the expandable coaptation element 20002 differently than the movement of the actuation portions or members 20008. By adjusting the actuation portions or members 20008 and the secondary control 20012, the expandable coaptation element can be adjusted to a variety of different shapes and sizes.

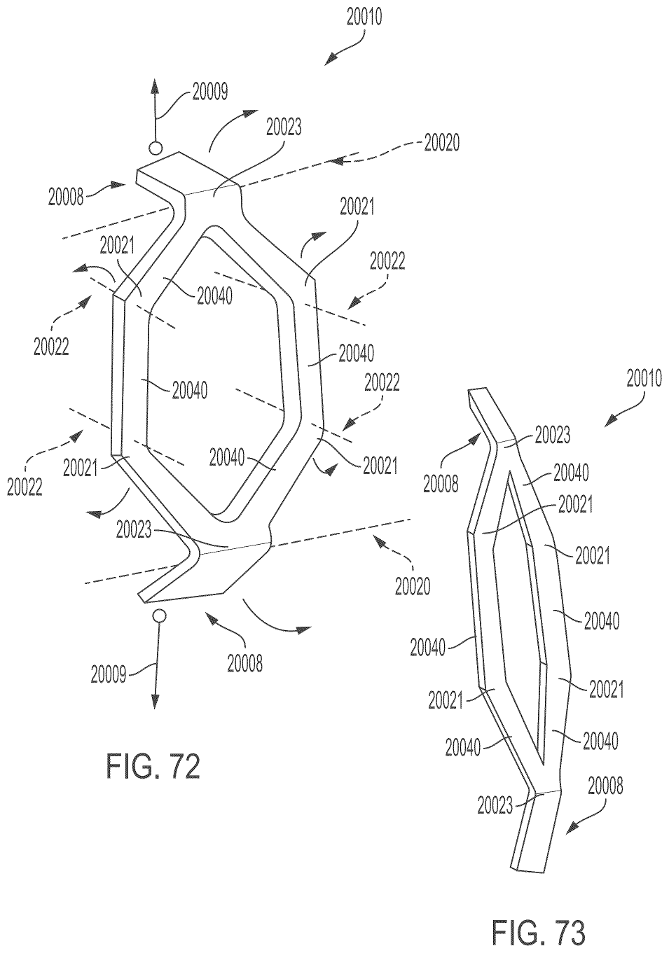

FIGS. 72-73 depict a control cell 20010 that forms part of the frame 20004 of the expandable coaptation element 20002. The control cell 20010 can expand by foreshortening, narrowing, and/or bending or flexing at certain points. The actuation portions or members 20008 are connected to the control cell 20010 via a plurality of tabs, connectors or connection portions 20006 that are coupled to the upstream and downstream ends of the cell at bending points 20023. In some implementations, the elastic properties of the material, variations in the width of the material, and/or variations the thickness of the material can be used to create the control cell 20010 with bending points 20021, 20023 that bend or flex before one another and/or before other portions of the control cell 20010 and/or bend or flex out of the plane of the control cell. In some implementations, the force applied to the control cell will initially overwhelm the bending points 20021 and/or the bending points 20023, causing the points to give and the shape of the expandable coaptation element 20002 to change.

For example, the bending points 20021, 20023 can be configured such that:
the points 20021 flex before the points 20023;
the points 20023 flex before the points 20021;
the points 20021 and the points 20023 simultaneously flex;
the points 20021 partially flex and then the points 20021 and the points 20023 flex; and/or
the points 20023 partially flex and then the points 20021 and the points 20023 flex.

FIG. 72 shows the control cell 20010 with bending points 20021 that bend about a plurality of bending axes 20022 and bending points 20023 that bend about axes 20020. A comparison of FIGS. 72 and 73 illustrates how the control cell 20010 can bend/be manipulated by adjusting the actuation portions or members 20008 in an upward or downward direction. In some implementations, by adjusting the actuation portions or members 20008 in an upward or downward direction, the bending point 20023 on the actuator will give first and elongate the control cell 20010. Then, the bending points 20021 will bend along the bending axes 20022, thus further elongating or shortening the control cell 20010. FIG. 73 depicts a control cell 20010 within a frame 20004 after the actuation portions or members 20008 have been manipulated in an upward and downward direction, thus elongating and narrowing the control cell 20010.

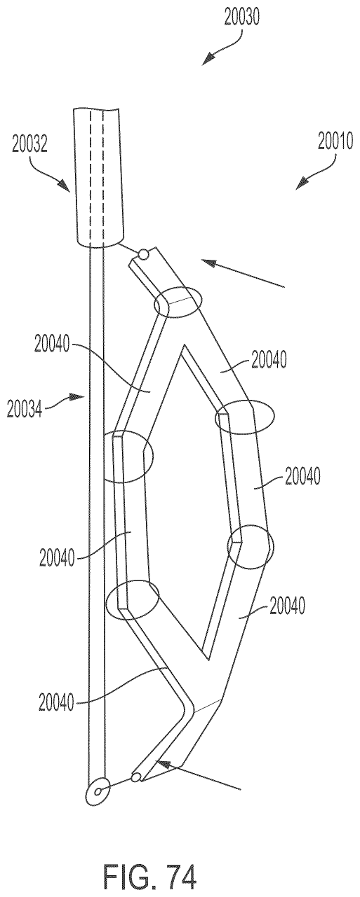

Control cells 20010 can be expanded and contracted in a variety of different ways. Any mechanism capable of moving the actuation members 20008 as illustrated by the arrows 20009 in FIG. 72 can be used. FIG. 74 shows a control cell 20010, as described in FIGS. 71-73 with an example actuation member 20030. The actuation member can comprise a catheter 20032 and rod 20034 to manipulate, in an upward and/or downward direction, and expand and/or contract the control cell 20010. When expanded and/or contracted, the catheter 20032 and rod 20034 causes a strut or a plurality of struts 20040 to elongate and/or contract, thus adjusting the size of the control cell 20010.

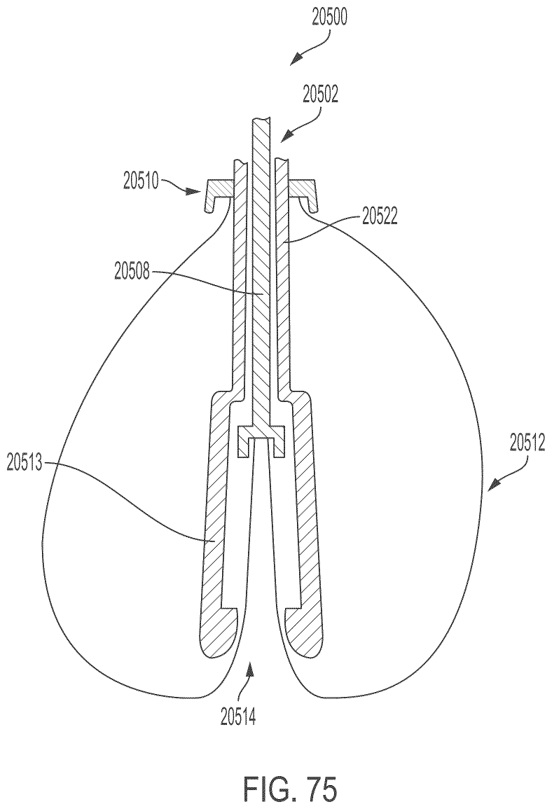

FIGS. 75-77 illustrate an example of an expandable coaptation element 20500. In the example illustrated by FIGS. 75-77, the expandable coaptation element 20500 can comprise one or more shape changing components 20512 and an adjustment mechanism 20502. The shape changing component 20512 is configured to change size and/or shape when a portion of the shape changing component 20512 is pulled into or pushed out of a receiver 20513 of the adjustment mechanism 20502.

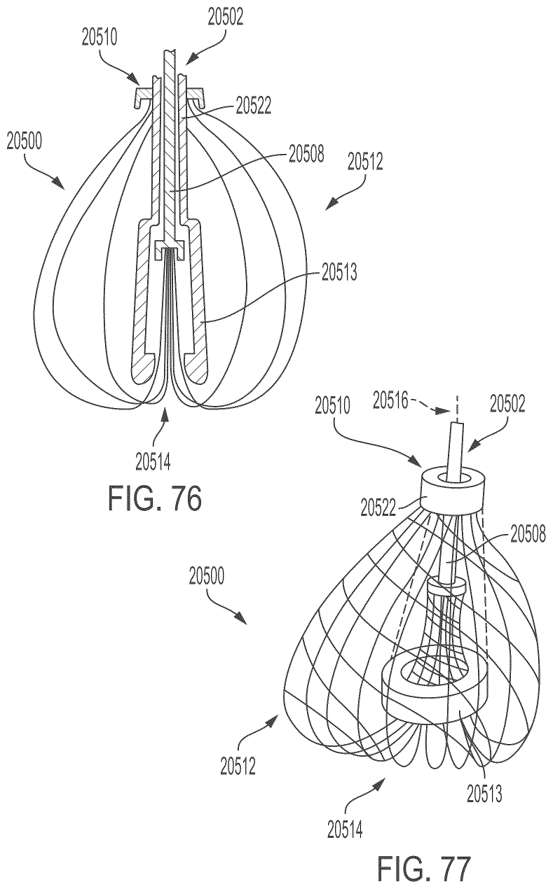

The shape changing components 20512 can take a wide variety of different forms having a variety of different expanded and contracted shapes. In the example illustrated by FIG. 76, the shape changing component 20512 can be made from individual wires having a whisk or whisk-like configuration. In the example illustrated by FIG. 77, the shape changing component 20512 can be made of a braided or mesh material. For example, the shape changing component 20512 can comprise a plurality of crossing wires. The shape changing components 20512 can have a variety of different shapes. For example, the shape changing components 20512 can be tear-drop shaped, spherical, cylindrical, conical, frustoconical, egg shaped, pyramid, a portion of a pyramid, diamond, combinations of these shapes, etc. in the expanded and/or retracted configurations. The shape changing components 20512 can be formed from a braided or woven tube of material—such as Nitinol wire—or any other flexible material suitable for implantation in the human body.

The shape changing components 20512 can be transformed from the expanded configuration to the compressed configuration in a wide variety of different ways. In the illustrated examples, the shape changing components are pulled into or pushed out of the receiver 20513 of the adjustment mechanism 20502. In the illustrated example, pushing the shape changing components 20512 out of the receiver 20513 causes the shape changing components 20512 to expand and drawing the shape changing components 20512 into the receiver causes the shape changing components to decrease in size. In some implementations, the shape changing components 20512 expand and contract laterally without increasing or substantially increasing the height of the expandable coaptation element 20500.

A variety of different mechanisms can be used to move the shape changing components 20512 from the expanded configuration to the contracted configuration. In some implementations, an inner shaft 20508 moves along an axis 20516 inside an outer shaft 20522 to move the shape changing components 20512 from the expanded configuration to the contracted configuration. In some implementations, a nut and a bolt can be used to move the shape changing components 20512 between expanded configuration and contracted configurations.

In the illustrated example, the shape changing component 20512 has a first end 20510 (e.g., proximal/upstream end) fixedly attached to the outer shaft 20522. The shape changing component 20512 has a distal end 20514 that is attached to the inner shaft 20508. The inner shaft 20508 and attached distal end 20514 are axially movably attached inside the receiver 20513. Axially adjusting the second or distal end 20514 within the receiver 20513 exposes more or less of the shape changing component 20512. The shape changing component 20512 can be biased to expand toward a generally teardrop or oval shape, such that exposure of more of the shape changing component 20512 makes the expandable coaptation element 20500 wider.

The expandable coaptation element 20500 can be used in a variety of different devices, including but not limited to, any of the treatment and/or repair devices disclosed in the present application. For example, any expandable coaptation element disclosed herein can be used in the device that is schematically illustrated in FIGS. 8-14. The expandable coaptation element 20500 can be positioned to engage leaflets 30, 32, 34 (see FIGS. 7 and 34) or leaflets 20, 22 (see FIGS. 6 and 36) as part of any suitable device (e.g., any treatment and/or repair device disclosed in the present application). The device can be deployed from a delivery sheath, a steerable catheter and/or an implant catheter. The device can include an expandable coaptation element 20500 and an anchor portion having two or more anchors, such as the anchor portion and anchors described herein. The device can be a prosthetic spacer device, valve repair device, valve treatment device, or another type of device that attaches to leaflets of a native valve.

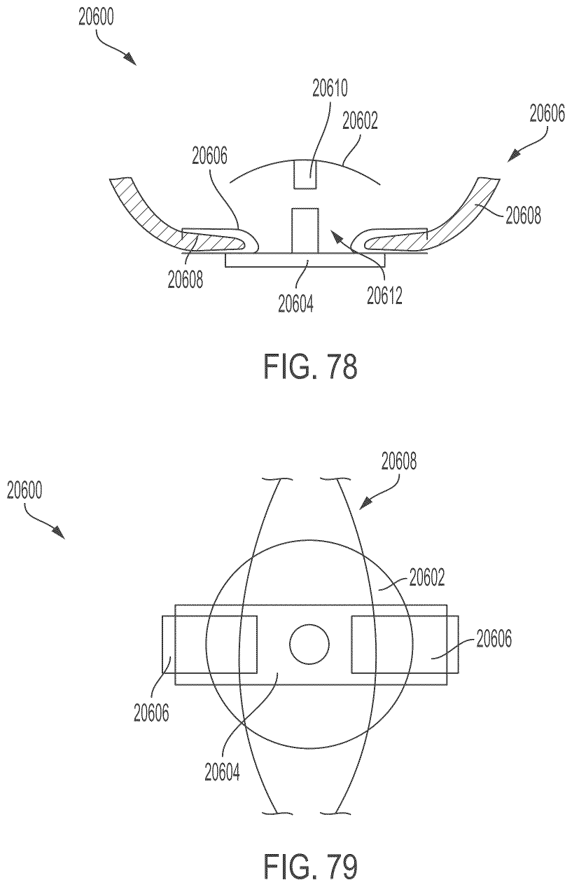

Referring to FIGS. 78-81, in some implementations, an extension or blocking member, etc. (e.g., a cap, umbrella, canopy, awning, cap-like extension, umbrella-like extension, canopy-like extension, etc.) can be attached to a device (e.g., a treatment device, a repair device, etc.) to block, divert or inhibit regurgitant blood flow through the native valve, such as a native mitral valve. The extension or blocking member can take a variety of different forms. The extension or blocking member can be sized and shaped to reduce regurgitation while maximizing flow from the atrium to the ventricle. For example, the extension or blocking member can be shaped to match or substantially match a profile of the regurgitant flow and/or have a shape that corresponds to an annulus of the native valve but is smaller than the annulus of the native valve.

Referring now to FIGS. 78-81, an example of a device 20600 (e.g., valve repair device, valve treatment device, implantable device, implant, etc.) is shown. The device 20600 (e.g., a treatment device, a repair device, etc.) can include extension or blocking member 20602 (e.g., cap, umbrella, canopy, awning, cap-like extension, umbrella-like extension, canopy-like extension, etc.), a frame or base 20604, and a clasp or a plurality of clasps 20606. The extension 20602 can be used to cover a gap between closed leaflets to block or partially block regurgitation through the native valve. The extension 20602 can be connected to the frame or base 20604 in a variety of different ways. In the illustrated example, the extension has a stem 20610 that is secured to a complimentary socket 20612 on the frame 20604. However, the extension 20602 can be attached to the frame or base 20604 in any manner or the extension 20602 can be connected directly to the one or more clasps 20606.

In some implementations, the extension 20602 is expandable. For example, the extension can take the form of or include features of an amplatzer, occluder or plug. In some implementations, the extension has a fixed size when deployed, but can be compressed to fit inside a delivery catheter. The extension 20602 can be composed of cloth, semi-rigid material, rigid material, or shape-memory alloy wire—such as Nitinol—to provide shape-setting capability, or any other flexible material suitable for implantation in the human body.

A clasp or a plurality of clasps 20606 can be attached to the frame 20604 to attach leaflets of the native valve 20608 to one another. In some implementations, the frame 20604 can be omitted. For example, the clasps 20606 can be connected to one another, be integrally formed, and/or connect directly to the extension 20602.

FIG. 79 shows a top plan view of the example device 20600 including the extension 20602. As can be seen in FIG. 79, the extension 20602 covers a gap between closed leaflets to block or partially block regurgitation through the native valve.

The extension 20602 can be used in a variety of different devices, including but not limited to, any of the treatment and/or repair devices disclosed in the present application. As one example, any extension, blocking member, etc. disclosed herein can be used in the device that is schematically illustrated in FIGS. 8-14. The device 20600 is one of the many different configurations that the device that is schematically illustrated in FIGS. 8-14 with an extension 20602 can take. The device 20600 can include any other features for a device (e.g., a treatment device, a repair device, etc.) discussed in the present application, and the extension 20602 can be positioned to block or impede regurgitant flow through leaflets 30, 32, 34 (see FIGS. 7 and 34) or leaflets 20, 22 (see FIGS. 6 and 36) as part of any suitable device (e.g., any treatment and/or repair device disclosed in the present application). The device 20600 can be deployed from a delivery sheath, a steerable catheter, and/or an implant catheter. The device 20600 can include an anchor portion having two or more anchors, such as the anchor portions and anchors described herein.

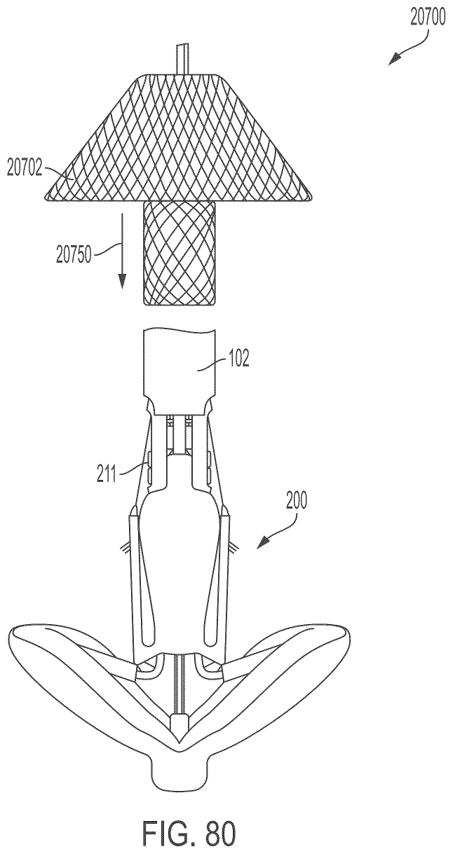

FIG. 80 shows an example valve system 20700 with an extension or blocking member 20702 (e.g., cap, umbrella, canopy, awning, cap-like extension, umbrella-like extension, canopy-like extension, etc.) being installed on a device 200 (e.g., a treatment device, a repair device, etc.). In the illustrated example, the device 200 can be the same as or similar to the device illustrated by FIG. 22. However, the extension 20702 can be installed on any device, such as any device (e.g., treatment and/or repair device, etc.) shown and described in the present application.

The extension 20702 can be installed on the device 200 in a variety of different ways. In the illustrated example, the extension 20702 is advanced as indicated by arrow 20750 over an implant catheter 102 that is used to deploy the device 200. Once in place, the extension 20702 is attached to the device 200. For example, the extension 20702 can be attached to the collar 211. However, the extension 20702 can be attached to the device 200 in any manner. The extension 20702 can be used to cover a gap that remains after the coaptation portion 204 of the device 200 is positioned. Covering the remaining gap can block or impede regurgitation through the native valve.

In some implementations, the extension 20702 is expandable. For example, the extension can take the form of or include features of and amplatzer occluder or plug. In some implementations, the extension has a fixed size when deployed, but can be compressed to fit inside a delivery catheter. The extension 20702 can be composed of cloth, semi-rigid material, rigid material, or shape-memory alloy wire—such as Nitinol—to provide shape-setting capability, or any other flexible material.

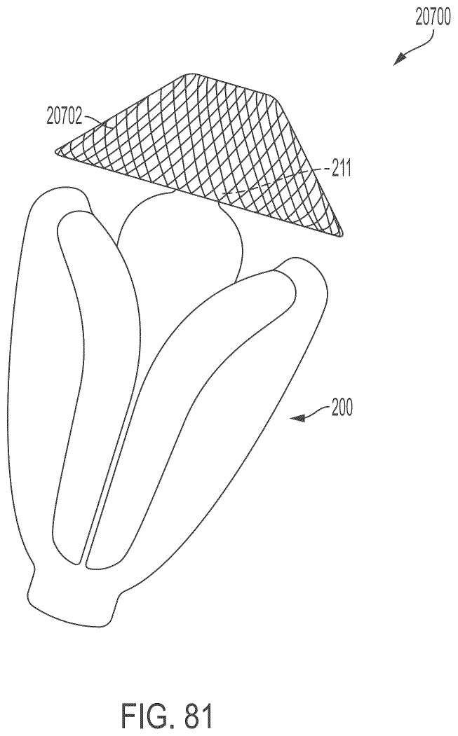

The extension 20702 can be inserted simultaneously with the device 200 or after the device 200 has been implanted. For example, the device can be positioned. Then, regurgitation can be assessed. Based on the remaining regurgitation, an extension 20702 can be selected and added to the device 200. FIG. 81 shows a device 200 with the extension 20702 secured.

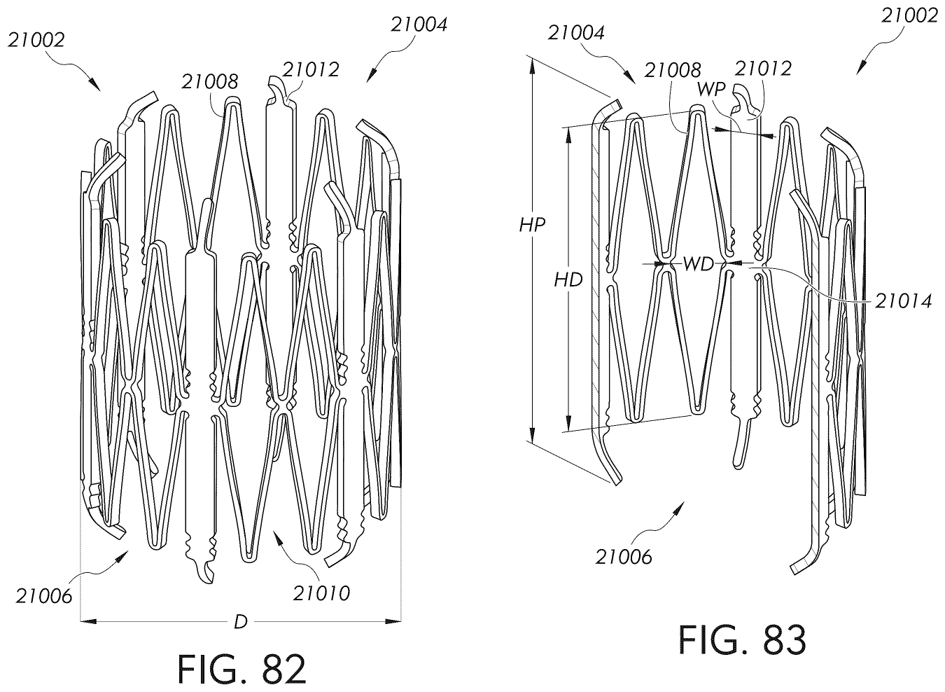

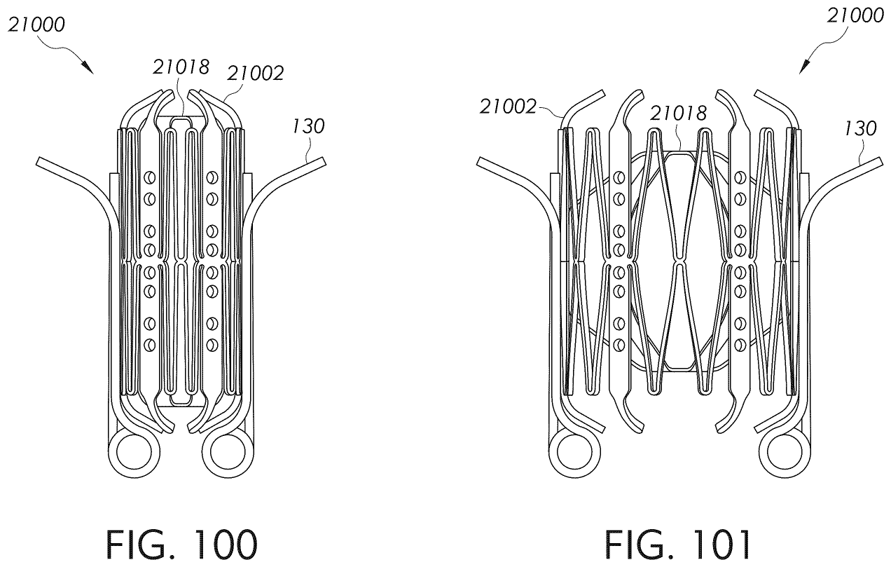

FIGS. 82-83 illustrate an example frame member 21002 (e.g., frame, body, cage, fixture, chassis, form, etc.) for an expandable coaptation element 21000 (e.g., FIG. 100-101). The expandable coaptation element 21000 (e.g., spacer, coaption element, gap filler, membrane, sheet, plug, wedge, balloon, etc.) can be part of a device (e.g., a treatment device, a repair device, etc.) for repairing a native heart valve and can be used as part of any device described herein.

In some implementations, the frame member 21002 is configured to move between a first radially contracted configuration and a second radially expanded configuration. The frame member 21002 can be configured in a variety of ways. In some implementations, the frame member 21002 has a generally cylindrical shape (i.e., a circular cross section) having a proximal end 21004, a distal end 21006 opposite the proximal end 21004, and a diameter D that can be collapsed (i.e., in the first configuration) and expanded (i.e., in the second configuration). In some implementations, however, the frame member 21002 can have a shape other than cylindrical (e.g., a cross section that is oval, rectangular, elliptical, or other suitable shape).

In some implementations, the frame member 21002 can include a plurality of interconnected struts 21008 that are configured to flex or bend to allow the frame member 21002 to move between the first and second configuration. The interconnected struts 21008 can be configured in a variety of ways, such as for example, the number and size of the struts, the shape of each strut, the arrangement of struts relative to other struts, the interconnection of the struts, etc. Any configuration that can facilitate the expansion and contraction of the frame member 21002 can be used. In some implementations, the struts 21008 are arranged in diamond patterns 21010 having a height HD and a width WD (FIG. 83).

In some implementations, the example frame member 21002 includes a plurality of posts 21012 interconnected by the plurality of struts 21008. The posts 21012 can be configured in a variety of ways, such as for example, the number and size of the posts, the shape of each post, the arrangement and connection of posts relative to the struts, etc.

In the illustrated example, each post 21012 extends linearly from the proximal end 21004 to the distal end 21006 and has a height HP and a width WP (FIG. 83). The width WP, in the illustrated example, is larger than a width WS of each strut. In some examples, the width WP is greater than twice, or greater than three times, the width WS of each strut 21008. In some implementations, the width WP of the post is less than or equal to the width WS of each strut.

In the illustrated example, each post 21012 is connected on either side, along a midpoint 21014, to one of the diamond patterns 21010 of struts 21008. In the illustrated example, the frame member 21002 includes six (6) posts 21012 evenly positioned around the periphery of the frame member 21002. Each of the six (6) posts 21012 is separated by two interconnected diamond patterns 21010 of the struts 21008. The posts 21012 and the struts 21008 can be interconnected in any suitable manner.

In some implementations, the frame member 21002 is formed as a unitary piece. For example, the frame member 21002 can be laser-cut from a tube. In other implementations, however, some of the struts 21008 and the posts 21012 can be formed separately and connected in any suitable manner (e.g., welded together).

In some implementations, the frame member 21002 can comprise a flexible material that can be a metal fabric, such as a mesh, woven, braided, or formed in any other suitable way or a laser cut or otherwise cut flexible material. The material can be cloth, shape-memory alloy wire—such as Nitinol—to provide shape-setting capability, or any other flexible material suitable for implantation in the human body.

In some implementations, when the diameter D of the frame member 21002 expands, the height HD of each of the diamond patterns 21010 decreases while the width WD increases. Conversely, the height HP of each post 21012 does not change. Thus, the frame member 21002 has a fixed height (i.e., the height HP of the posts) that does not change between the first and second configuration.

In some implementations, the frame member 21002 can be configured to change height between the first and the second configuration. For example, the posts can be replaced with diamond patterns, such that the entire frame member 21002 decreases in height as the frame member increases in width.

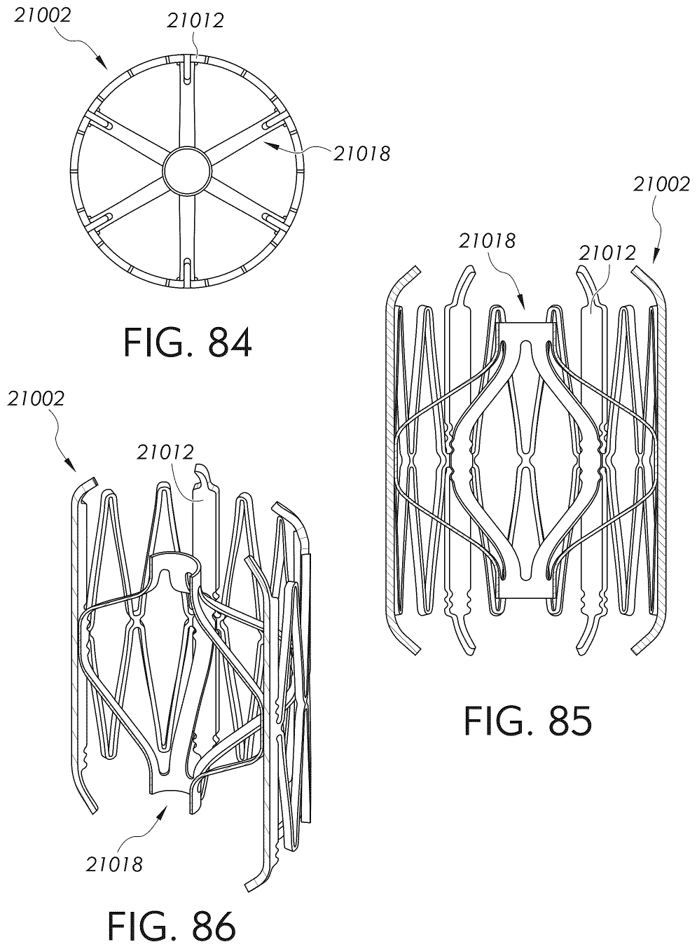

The frame member 21002 can be moved between the first and the second configurations by any suitable means. For example, the frame member 21002 can be mounted around an expandable mechanism 21016 (see FIGS. 84-86) configured to engage the frame member 21002 to move the frame member 21002 between the first and the second configurations. A variety of different mechanisms can be used to move the frame member 21002 between the first and second configurations. For example, any expandable mechanism disclosed herein can be used. The expandable mechanism can comprise one or more of a balloon, expandable container, expandable material, self-expanding material, self-expanding frame, stent, mechanically-expandable frame, pivoting and/or scissoring extensions and/or struts, expanding pully mechanism, Hoberman mechanism, cam, worm screw, rack and pinion, foam, etc.

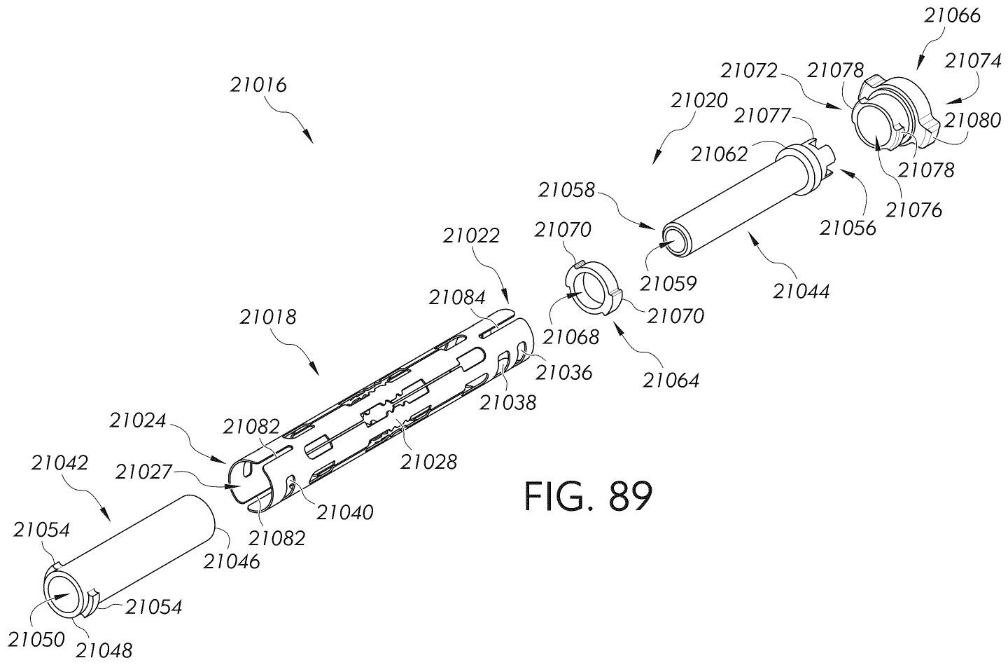

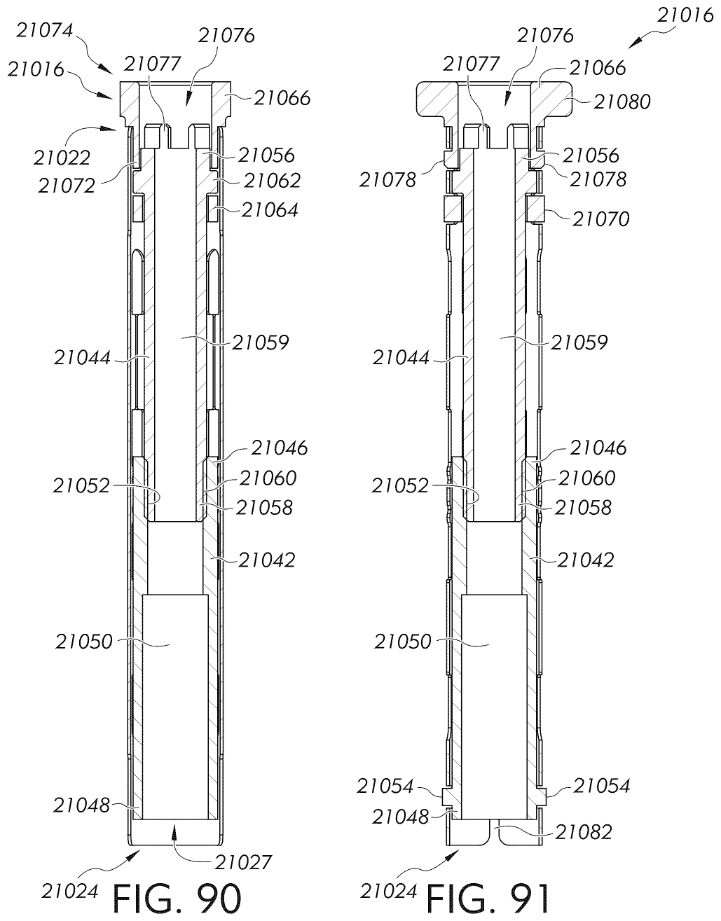

In some implementations, as shown in FIG. 89, the expandable mechanism 21016 includes an expandable member or expansion member 21018 (e.g., frame, body, strut assembly, tube, shaft, etc.) and an actuation mechanism. Referring to FIGS. 84-86, in some implementations, the frame member 21002 is mounted around the expandable/expansion member 21018. The expandable/expansion member 21018 can be configured in a variety of ways. Any configuration capable of moving the frame member 21002 between the first and the second configuration can be used.

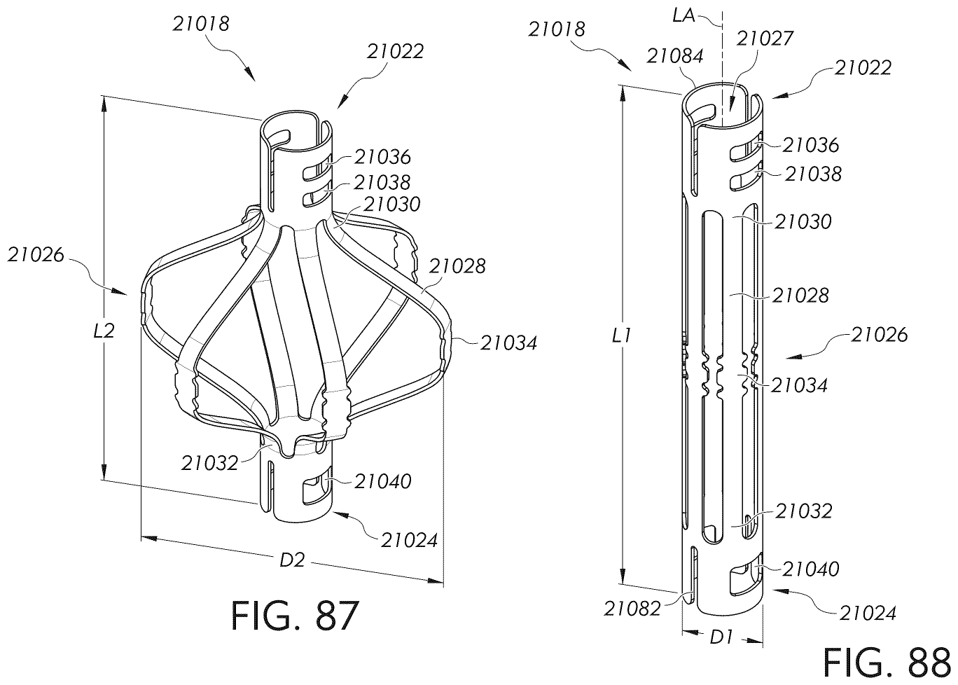

Referring to FIGS. 87-88, in some implementations, the expandable/expansion member 21018 is configured as an elongated cylindrical shape having a proximal end portion 21022, a distal end portion 21024 opposite the proximal end portion 21022, an intermediate portion 21026 connecting the proximal end portion 21022 and the distal end portion 21024, and a passage 21027 extending through the expandable/expansion member 21018 from the proximal end portion 21022 to the distal end portion 21024.

In some implementations, the intermediate portion 21026 is formed from a plurality of strips 21028 that are configured to bend or flex. In some implementations, each strip 21028 has a proximal end 21030 attached to the proximal end portion 21022 of the expandable/expansion member 21018, a distal end 21032 attached to the distal end portion 21024, and an intermediate portion 21034 between the proximal end 21030 and the distal end 21032.

In some implementations, the expandable/expansion member 21018 includes six (6) strips 21028 equally spaced apart around the periphery of the expandable/expansion member 21018. In some implementations, however, the strips 21028 may not be equally spaced and/or the intermediate portion 21026 can include more or less than six (6) strips.

In some implementations, the expandable/expansion member 21018 is movable between a first configuration (i.e., collapsed or narrow configuration) (FIG. 88) and a second configuration (i.e., an expanded or wide configuration) (FIG. 87). As shown in FIG. 88, in the first configuration, each of the plurality of strips 21028 extend parallel to a longitudinal axis LA of the expandable/expansion member 21018 and the expandable/expansion member 21018 has a first length L1. In the first configuration, the proximal end portion 21022, the distal end portion 21024, and the intermediate portion 21026 have the same first diameter D1.

In some implementations, the expandable/expansion member 21018 can be formed as a unitary piece. For example, the expandable/expansion member 21018 can be laser-cut from a tube. In some implementations, the expandable/expansion member 21018 can be formed from multiple pieces, such as for example, a pair of Nitinol sheets.

In some implementations, the expandable/expansion member 21018 can comprise a flexible material that can be a metal fabric, such as a mesh, woven, braided, or formed in any other suitable way or a laser cut or otherwise cut flexible material. The material can be cloth, shape-memory alloy wire—such as Nitinol—to provide shape-setting capability, or any other flexible material suitable for implantation in the human body.

In some implementations, as shown in FIG. 87, in the second configuration, the proximal end portion 21022 is moved toward the distal end portion 21024, or vice versa, causing the strips 21028 to bend or flex outward such that the intermediate portion 21026 expands to a second diameter D2 that is greater than the first diameter D1. Further, in second configuration, the expandable/expansion member 21018 has a second length L2, less than the first length L1 and the diameter of the proximal end portion 21022 and the distal end portion 21024 remain at the first diameter D1 while the intermediate portion 21026 expands to the second diameter D2.

As shown in FIGS. 84-86, in some implementations, the expandable/expansion member 21018 positioned within the periphery of the frame member 21002 to engage the frame member 21002. In some implementations, the intermediate portion 21034 of each strip 21028 is attached to the frame member 21002. In some implementations, the intermediate portion 21034 of each strip 21028 is attached to a corresponding post 21012 of the frame member 21002. The intermediate portion 21034 of each strip 21028 can attach to the frame member 21002 in any suitable manner, such as for example, welding.

In some implementations, as a result of the expandable/expansion member 21018 being positioned within the periphery of the frame member 21002 and attached to the frame member 21002, movement of the expandable/expansion member 21018 between the first configuration (i.e., collapsed or narrow configuration) (FIG. 88) and a second configuration (i.e., expanded or wide configuration) (FIG. 87) moves the frame member 21002 between the frame member's first configuration (i.e., collapsed) and second configuration (i.e., expanded).

In some implementations, the frame member 21002 and/or the expandable/expansion member 21018 can be configured to be normally in the first configuration (i.e., collapsed or narrow), in the second configuration (i.e., expanded or wide), or some intermediate position between the first and the second configuration. Thus, in some implementations, movement of the expandable/expansion member 21018 pulls the frame member 21002 inward toward the first position against a bias of the frame member 21002. In some implementations, movement of the expandable/expansion member 21018 pushes the frame member outward to the second position against a bias of the frame member 21002.

In some implementations, the actuation mechanism 21020 is configured to move the expandable/expansion member 21018 between the first and the second configurations. The actuation mechanism 21020 can be configured in a variety of ways. Any actuation mechanism capable of moving the expandable/expansion member 21018 between the first and the second configurations can be used. In some implementations, the actuation mechanism 21020 is mounted within the expandable/expansion member 21018 between the proximal end portion 21022 and the distal end portion 21024 of the expandable/expansion member 21018.

Referring to FIGS. 87-88, in some implementations, the expandable/expansion member 21018 can include structure configured to mount the actuation mechanism 21020 within the expandable/expansion member 21018. The structure can be configured in a variety of ways. In some implementations, the proximal end portion 21022 of the expandable/expansion member 21018 includes a first pair of radially opposed openings 21036, and a second pair of radially opposed openings 21038 distal to the first pair of radially opposed openings 21036 and the distal end portion 21024 of the expandable/expansion member 21018 includes a third pair of radially opposed openings 21040.

In some implementations, the actuation mechanism 21020 is configured as a threaded connection. Referring to FIG. 89, the example actuation mechanism 21020 includes a distal member 21042 (e.g., tube, body, shaft, etc.) configured to threadably couple with a proximal member 21044 (e.g., tube, body, shaft, etc.). The distal member 21042 and the proximal member 21044 can be configured in a variety of ways. In the illustrated example, the distal member 21042 is formed as a cylindrical tube having a circular cross section. In some implementations, however, the distal member 21042 can have a shape other than cylindrical (e.g., a cross section that is oval, rectangular, elliptical, or other suitable shape).

In some implementations, the distal member 21042 includes a proximal end 21046, a distal end 21048 opposite the proximal end 21046, and an inner passage 21050 extending through the distal member 21042 member from the proximal end 21046 to the distal end 21048. The inner passage 21050 includes female threads 21052 (FIGS. 90-91) for threadably coupling with the proximal member 21044.

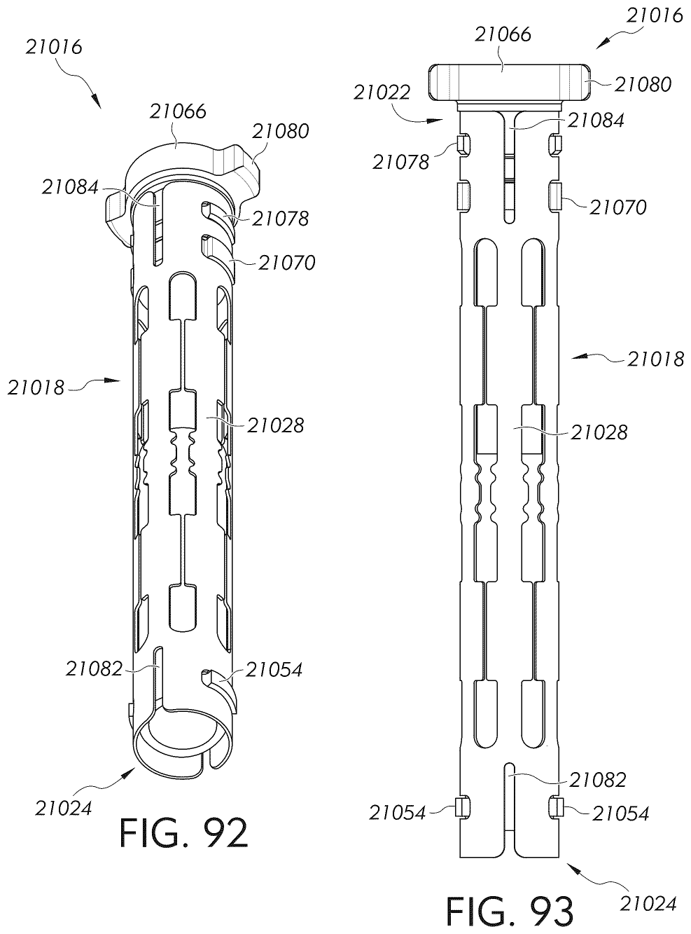

In some implementations, the distal member 21042 is sized to be received within the passage 21027 of the expandable/expansion member 21018. In some implementations, the distal member 21042 includes structure of fixing the distal member 21042 in position within the passage 21027 relative to the expandable/expansion member 21018. In the illustrated example, the distal end 21048 includes a pair of opposed projections 21054 configured to be received within the third pair of radially opposed openings 21040, as shown in FIGS. 90-93.

In some implementations, the proximal member 21044 is formed as a cylindrical tube having a circular cross section. In some implementations, the proximal member 21044 can have a shape other than cylindrical (e.g., a cross section that is oval, rectangular, elliptical, or other suitable shape).

In some implementations, the proximal member 21044 includes a proximal end 21056, a distal end 21058 opposite the proximal end 21056, and an inner passage 21059 extending through the proximal member 21044 from the proximal end 21056 to the distal end 21058.

In some implementations, the proximal member 21044 can include male threads 21060 (FIGS. 90-91) extending along at least a portion of the exterior of the proximal member 21044. The male threads 21060 are configured to mate with the female threads 21052 of the distal member 21042.

In some implementations, the proximal member 21044 is sized to be received within the passage 21027 of the expandable/expansion member 21018. In some implementations, the actuation mechanism 21020 can include structure for restricting axial movement of the proximal member 21044 within the passage 21027 relative to the expandable/expansion member 21018.

In some implementations, the structure restricts axial movement of the proximal member 21044 within the passage 21027 relative to the expandable/expansion member 21018 while allowing rotational movement of the proximal member 21044 relative to the expandable/expansion member 21018. The structure for restricting axial movement of the proximal member 21044 can be configured in a variety of ways.

In some implementations, the actuation mechanism 21020 includes a circumferential, radially extending ridge 21062 at the proximal end 21056. In some implementations, the ridge 21062 is configured to engage with a stop 21064 and an end cap 21066 to restrict axial movement of the proximal member 21044 within the passage 21027. The stop 21064 can be configured in a variety of ways. In some implementations, the stop 21064 is configured to inhibit movement of the proximal member 21044 distally within the passage 21027.

In some implementations, the stop 21064 is formed as a ring having a central passage 21068 sized to receive a portion of the proximal member 21044 therethrough. In some implementations, the stop 21064 is further sized to be received within the passage 21027.

In some implementations, the stop 21064 can include structure configured to fix the position of the stop 21064 relative to the expandable/expansion member 21018. In some implementations, the stop 21064 includes a pair of opposed projections 21070 configured to be received within the second pair of radially opposed openings 21038 of the expandable/expansion member 21018, as shown in FIGS. 90-93.

The end cap 21066 can be configured in a variety of ways. In some implementations, the end cap 21066 is configured to inhibit movement of the proximal member 21044 proximally within the passage 21027. In some implementations, the end cap 21066 is formed as a ring having a distal end 21072, a proximal end 21074 opposite the distal end 21072, and a central passage 21076 extending through the end cap 21066 from the distal end 21072 to the proximal end 21074.

In some implementations, the central passage 21076 is sized to receive at least a portion of the proximal end 21056 of the proximal member 21044. In some implementations, the proximal end 21056 of the proximal member 21044 can further include one or more engagement surfaces 21077 accessible through the central passage 21076 of the end cap 21066. In some implementations, the one or more engagement surfaces 21077 are configured to be engaged to rotate the proximal member 21044.

In some implementations, the end cap 21066 is further sized to be received within the passage 21027. In some implementations, the end cap 21066 can include structure configured to fix the position of the end cap 21066 relative to the expandable/expansion member 21018.

In some implementations, the end cap 21066 includes a pair of opposed projections 21078 at the distal end 21072 that are configured to be received within the first pair of radially opposed openings 21036 of the expandable/expansion member 21018, as shown in FIGS. 90-93.

In some implementations, the end cap 21066 can include a radial flange 21080 at the proximal end 21074 configured to inhibit over insertion of the end cap 21066 into the passage 21076.

Referring to FIGS. 90-93, in some implementations, when assembled, the actuation mechanism 21020 can move the expandable/expansion member 21018 between the first and the second configurations. In particular, the distal member 21042 is received within the passage 21027 of the expandable/expansion member 21018.

In some implementations, the pair of opposed projections 21054 on the distal member 21042 are received within the third pair of radially opposed openings 21040 thus both axially and rotationally fixing the distal member 21042 relative to the expandable/expansion member 21018.

In some implementations, the distal end portion 21024 of the expandable/expansion member 21018 and/or the distal member 21042 can be configured to move or flex to allow the pair of opposed projections 21054 to be positioned adjacent the third pair of radially opposed openings 21040.

In some implementations, the distal end portion 21024 of the expandable/expansion member 21018 includes one or more open-ended longitudinal slots 21082 that allow the distal end portion 21024 to flex outward as the pair of opposed projections 21054 are moved into position to be received within the third pair of radially opposed openings 21040. In some implementations, once the pair of opposed projections 21054 are received within the third pair of radially opposed openings 21040, the distal end portion 21024 returns back to its normal state.

In some implementations, the stop 21064 is received within the passage 21027 such that the pair of opposed projections 21070 are received within the second pair of radially opposed openings 21038 of the expandable/expansion member 21018. In some implementations, the proximal end portion 21022 of the expandable/expansion member 21018 and/or the stop 21064 can be configured to move or flex to allow the pair of opposed projections 21070 to be positioned adjacent the second pair of radially opposed openings 21038.

In some implementations, the proximal end portion 21022 of the expandable/expansion member 21018 includes one or more open-ended longitudinal slots 21084 that allow the proximal end portion 21022 to flex outward as the pair of opposed projections 21070 are moved into position to be received within the second pair of radially opposed openings 21038. In some implementations, once the pair of opposed projections 21070 are received within the second pair of radially opposed openings 21038, the proximal end portion 21022 returns back to its normal state.

In some implementations, the proximal member 21044 is received within the passage 21027 proximate to the distal member 21042. In some implementations, the distal end 21058 of the proximal member 21044 is received in the passage 21050 in the proximal end 21046 of the distal member 21042 such that the male threads 21060 of the proximal member 21044 threadably engage the female threads 21052 of the distal member 21042. In some implementations, the proximal member 21044 is threaded into the distal member 21042 until the ridge 21062 engages the stop 21064.

In some implementations, the distal end 21072 of the end cap 21066 is received within the passage 21027 such that the pair of opposed projections 21078 are received within the first pair of radially opposed openings 21036 of the expandable/expansion member 21018. The proximal end portion 21022 of the expandable/expansion member 21018 and/or the end cap 21066 can be configured to move or flex to allow the pair of opposed projections 21078 to be positioned adjacent the first pair of radially opposed openings 21036.

In some implementations, the proximal end portion 21022 of the expandable/expansion member 21018 includes the open-ended longitudinal slots 21084 that allow the proximal end portion 21022 to flex outward as the pair of opposed projections 21078 are moved into position to be received within the first pair of radially opposed openings 21036. In some implementations, once the pair of opposed projections 21078 are received within the first pair of radially opposed openings 21036, the proximal end portion 21022 returns back to its normal state.

As shown in FIGS. 90-91, in some implementations, when assembled, the ridge 21062 is sandwiched between the end cap 21066 and the stop 21064 such that axial movement of the proximal member 21044 relative to the proximal end portion 21022 of the expandable/expansion member 21018 is inhibited. Rotational movement of the proximal member 21044, however, is allowed.

In some implementations, a rotational device or tool (not shown) can engage the one or more engagement surfaces 21077 via the passage 21076 in the end cap 21066 to rotate the proximal member 21044 in a first rotational direction (i.e., to thread the proximal member 21044 into the distal member 21042). With the proximal member 21044 axially fixed to the proximal end portion 21022 of the expandable/expansion member 21018 and the distal member 21042 axially fixed to the distal end portion 21024 of the expandable/expansion member 21018, threading the proximal member 21044 into the distal member 21042 draws the proximal end portion 21022 of the expandable/expansion member 21018 toward the distal end portion 21024. As a result, the strips 21028 of the intermediate portion 21026 bend or flex outward. Since the intermediate portion 21034 of the strips 21028 are attached to the posts 21012 of the frame member 21002, the outward expansion of the strips 21028 causes the outward expansion of the frame member 21002.

Conversely, in some implementations, rotating the proximal member 21044 in a second rotational direction (i.e., to thread the proximal member 21044 out of the distal member 21042) pushes the proximal end portion 21022 of the expandable/expansion member 21018 away from the distal end portion 21024. As a result, the strips 21028 of the intermediate portion 21026 are pulled inward causing the inward contraction of the frame member 21002.

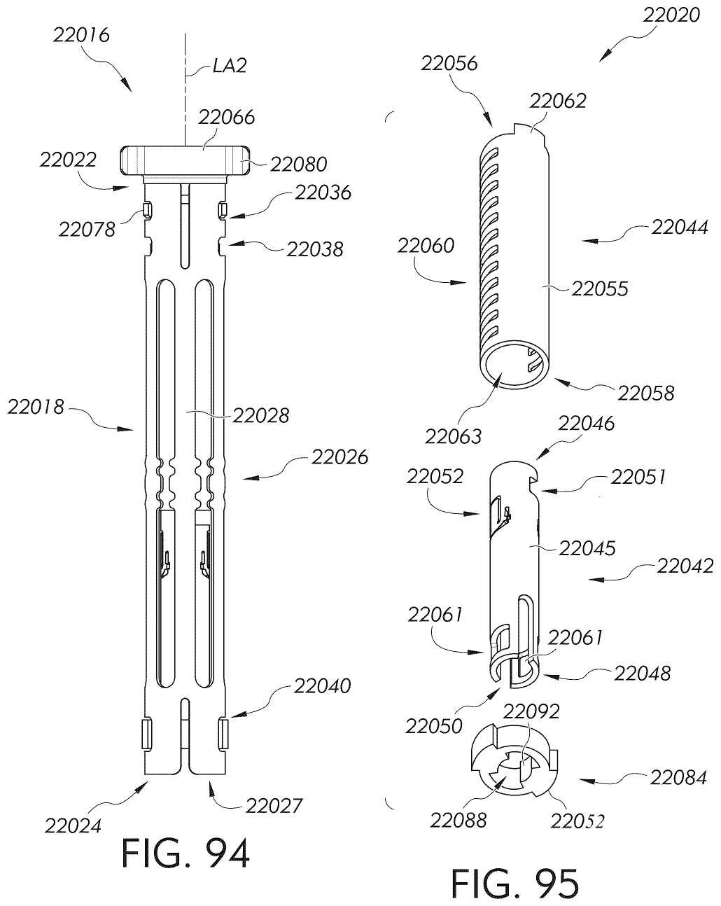

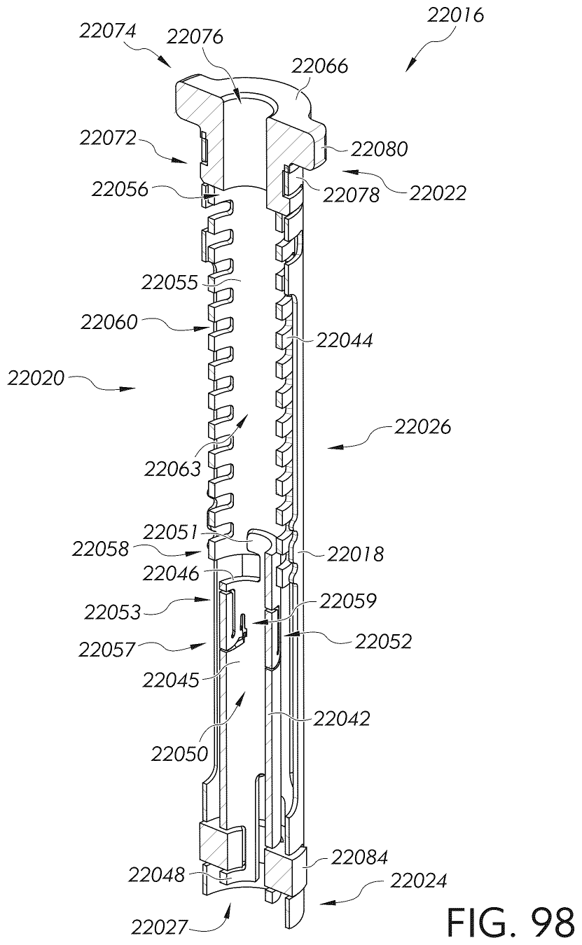

FIGS. 94-98 illustrate an example expandable mechanism 22016 usable in an expandable coaptation element. The expandable mechanism can be the same as or similar to any of the expandable mechanisms described anywhere in this disclosure. The expandable mechanism can comprise one or more of a balloon, expandable container, expandable material, self-expanding material, self-expanding frame, stent, mechanically-expandable frame, pivoting and/or scissoring extensions and/or struts, expanding pully mechanism, Hoberman mechanism, cam, worm screw, rack and pinion, foam, etc.

In some implementations, the expandable mechanism 22016 can be positioned and/or mounted inside an expandable frame member, such as the frame member 21002 (FIG. 82-83). The example expandable mechanism 22016 includes an expandable/expansion member 22018 and an actuation mechanism 22020 (FIGS. 94-95)

The expandable/expansion member 22018 can be configured in a variety of ways. In the illustrated example, the expandable/expansion member 22018 is substantially similar to the expandable/expansion member 21018 of FIGS. 87-88, thus the description of the expandable/expansion member 21018 applies equally to the expandable/expansion member 22018.

In some implementations, the expandable/expansion member 22018 is configured as an elongated cylindrical shape having a proximal end portion 22022, a distal end portion 22024, an intermediate portion 22026, and a passage 22027 extending through the expandable/expansion member 22018 from the proximal end portion 22022 to the distal end portion 22024.

In some implementations, the intermediate portion 22026 is formed from a plurality of strips 22028 (FIG. 94) that are configured to bend or flex.

In some implementations, the expandable/expansion member 22018 can include structure configured to mount the actuation mechanism 22020 within the expandable/expansion member 22018. In some implementations, the proximal end portion 22022 of the expandable/expansion member 22018 includes a first pair of radially opposed openings 22036, and a second pair of radially opposed openings 22038 distal to the first pair of radially opposed openings 22036 and the distal end portion 22024 of the expandable/expansion member 22018 includes a third pair of radially opposed openings 22040.

In some implementations, the expandable/expansion member 22018 is movable between a first configuration (i.e., collapsed or narrow configuration) and a second configuration (i.e., expanded or wide configuration). As shown in FIG. 94, in some implementations, in the first configuration, each of the plurality of strips 22028 extend parallel to a longitudinal axis LA2 of the expandable/expansion member 22018 and, in the second configuration (see FIG. 87), the strips 22028 bend or flex outward such that the intermediate portion 22026 expands radially outward.

In some implementations, the actuation mechanism 22020 is configured to move the expandable/expansion member 22018 between the first and the second configurations. The actuation mechanism 22020 can be configured in a variety of ways. Referring to FIGS. 95-98, in some implementations, the actuation mechanism 22020 includes a distal member 22042 and a proximal member 22044.

The distal member 22042 and the proximal member 22044 can be configured in a variety of ways. In the illustrated example, the distal member 22042 is formed as a cylindrical tube having a sidewall 22045 with a circular cross section. In some implementations, however, the distal member 22042 can have a shape other than cylindrical (e.g., a cross section that is oval, rectangular, elliptical, or other suitable shape).

In some implementations, the distal member 22042 includes a proximal end 22046, a distal end 22048 opposite the proximal end 22046, and an inner passage 22050 extending through the distal member 22042 member from the proximal end 22046 to the distal end 22048. In some implementations, the proximal end 22046 includes a coupling portion 22051 configured to couple to an actuation element (e.g., actuation shaft, actuation rod, actuation tube, actuation wire, etc.) (not shown).

In some implementations, the distal member 22042 includes one or more locking members 22052 configured to engage the proximal member 22044 to lock the position of the distal member 22042 relative to the proximal member 22044. The one or more locking members 22052 can be configured in a variety of ways.

In the illustrated example, each locking member 22052 is formed from the side wall 22045. For example, each locking member 22052 can be formed from laser cuts through the side wall 22045. Each locking member 22052 can include a first end 22053, a second end 22057 opposite the first end 22053, and an intermediate portion 22063 between the first end 22053 and the second end 22057.

In some implementations, each locking member 22052 is configured to pivot about the intermediate portion 22059 between a first position in which the locking member 22052 is parallel with the side wall 22045, as shown in FIGS. 95-98, and a second position (not shown) in which the locking member 22052 pivots such that the first end 22053 extends external to the side wall 22045 and the second end 22057 extends into the passage 22050.

In some implementations, each locking member 22052 is biased to the second position. For example, at least a portion of the distal member 22042 can include a shape memory alloy and the one or more locking members 22052 can be shape set to the second position.

In some implementations, the distal member 22042 is sized to be received within the passage 22027 of the expandable/expansion member 21018. In some implementations, the distal member 22042 can include structure for fixing the distal member 22042 in position within the passage 22027 relative to the expandable/expansion member 22018. In the illustrated example, the distal end 22048 includes a pair of apertures 22061.

In some implementations, the proximal member 22044 is formed as a cylindrical tube having a sidewall 22055 with a circular cross section. In some implementations, however, the proximal member 22044 can have a shape other than cylindrical (e.g., a cross section that is oval, rectangular, elliptical, or other suitable shape).

In some implementations, the proximal member 22044 includes a proximal end 22056, a distal end 22058 opposite the proximal end 22056, and an inner passage 22063 extending through the proximal member 22044 from the proximal end 22056 to the distal end 22058. In some implementations, the proximal member 22044 includes structure configured to interact with the one or more locking members 22052 of the distal member 22042. In some implementations, the structure is formed as two series of slots 22060 formed in the sidewall 22055 on opposite sides of each other. Each slot 22060 can be arranged transverse (e.g., perpendicular) to the longitudinal axis LA2 and each series of slots 22060 can be aligned with a locking member 22052.

In some implementations, the proximal member 22044 is sized to be received within the passage 22027 of the expandable/expansion member 22018. In some implementations, the proximal member 22044 includes structure for restricting rotational movement of the proximal member 22044 within the passage 22027 relative to the expandable/expansion member 22018. The structure for restricting axial movement of the proximal member 21044 can be configured in a variety of ways. In some implementations, the proximal member 22044 includes one or more tabs 22062 extending axially from the proximal end 22056.

In some implementations, the actuation mechanism 22020 includes an end cap 22066. The end cap 22066 can be configured in a variety of ways. In some implementations, the end cap 22066 is configured to inhibit movement of the proximal member 22044 proximally and rotationally within the passage 22027.

In some implementations, the end cap 22066 is formed as a ring having a distal end 22072, a proximal end 22074 opposite the distal end 22072, and a central passage 22076 extending through the end cap 22066 from the distal end 22072 to the proximal end 22074. In some implementations, the end cap 22066 is configured to receive each of the one or more tabs 22062 in a groove or recess 22077 to inhibit rotation of the proximal member 22044 within the passage 22027.

In some implementations, the distal end 22072 of the end cap 22066 is sized to be received within the passage 22027. In some implementations, the end cap 22066 can include structure configured to fix the position of the end cap 22066 relative to the expandable/expansion member 22018.

In some implementations, the end cap 22066 includes a pair of opposed projections 22078 at the distal end 22072 that are configured to be received within the first pair of radially opposed openings 22036 of the expandable/expansion member 22018, as shown in FIGS. 90-93. In some implementations, the end cap 22066 can include a radial flange 22080 at the proximal end 22074 configured to inhibit over insertion of the end cap 22066 into the passage 22076.

In some implementations, the actuation mechanism 22020 includes a stop 22084 (FIG. 95) to restrict movement of the distal member 22042 relative to the expandable/expansion member 22018. The stop 22084 can be configured in a variety of ways. In some implementations, the stop 22084 is formed as a ring having a central passage 22088 sized to receive the distal end 22048 of the distal member 22042 therein.

In some implementations, the stop 22084 is further sized to be received within the passage 22027 of the expandable/expansion member 22018. In some implementations, the stop 22084 can include structure configured to fix the position of the distal member 22042 relative to the expandable/expansion member 22018. In some implementations, the stop 22084 includes a pair of opposed first projections 22090 extending radially outward and configured to be received within the third pair of radially opposed openings 22040 of the expandable/expansion member 22018, as shown in FIGS. 94 and 97. In some implementations, the stop 22084 includes a pair of opposed second projections 22092 extending radially inward and configured to be received within the pair of apertures 22061 at the distal end 22048 of the distal member 22042.

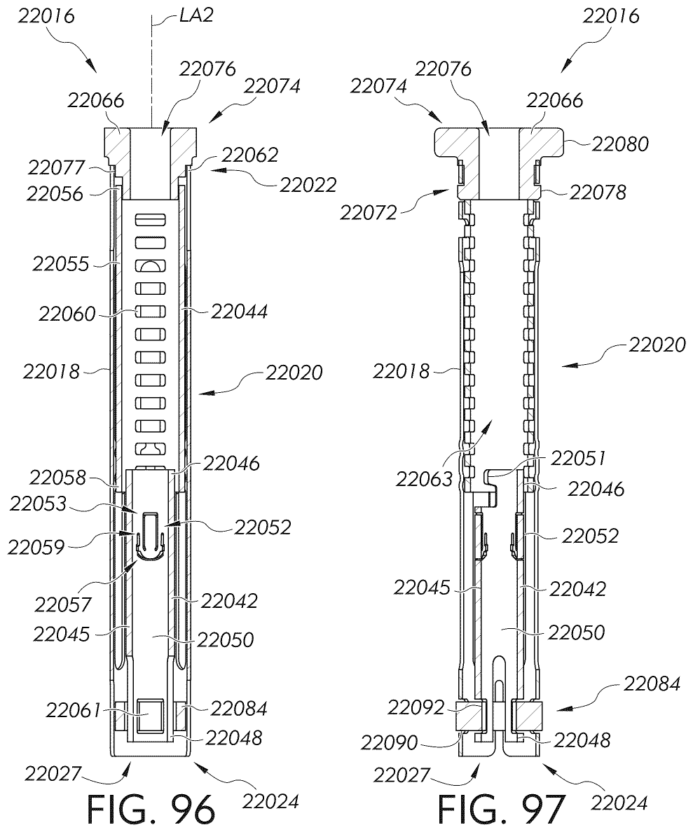

In some implementations, when assembled, the actuation mechanism 22020 can move the expandable/expansion member 22018 between the first and the second configurations. Referring to FIGS. 96-98, the distal member 22042 is received within the passage 22027 of the expandable/expansion member 21018. In some implementations, the stop 22084 is positioned within the 22027 such that the pair of opposed first projections 22090 are received within the third pair of radially opposed openings 22040 of the expandable/expansion member 22018 and the pair of opposed second projections are received within the pair of apertures 22061 at the distal end 22048 of the distal member 22042. As a result, the distal member 22042 is held in position relative to the expandable/expansion member 22018.

Further, in some implementations, the proximal member 22044 is received within the passage 22027 of the expandable/expansion member 21018 and the proximal end 22046 of the distal member 22042 is received within the passage 22063 of the proximal member 22044. In some implementations, the distal end 22072 of the end cap 22066 is secured to the proximal member.

In some implementations, the distal end 22072 of the end cap 22066 can be received within the passage 22027 such that the projections 22078 of the end cap 22066 are received in the first pair of radially opposed openings 22036 of the expandable/expansion member 22018. As a result, the end cap 22066 fixes the proximal member 22044 at the proximal end portion 22022 of the expandable/expansion member 22018.

In some implementations, the proximal member 22044 is separate from the end cap 22066 and the proximal member 22044 is separately attached to the expandable/expansion member 22014. For example, the proximal member 22044 can be secured to the expandable/expansion member 22014 in the same or similar manner that the proximal member 21044 is attached to the expandable/expansion member 21014 in the implementation illustrated by FIGS. 89-93.

In some implementations, the device (e.g., device 100 or another device herein) can be delivered by a delivery system (e.g., delivery system 102). In some implementations, the delivery system can include a first actuation element (e.g., actuation shaft, actuation rod, actuation tube, actuation wire, etc.) (not shown) that extends through the passage 22076 in the end cap 22066, through the passage 22063 in the proximal member 22044, and couples to the coupling portion 22051 at the proximal end 22046 of the distal member 22042.

In some implementations, the delivery system can include an optional second actuation element (e.g., actuation shaft, actuation rod, actuation tube, actuation wire, etc.) (not shown) that extends coaxially through the first actuation element (not shown) and into the passage 22050 of the distal member 22042.

In some implementations, with the first and/or second actuation member (not shown) in position within the passage 22050 adjacent the one or more locking members 22052, the locking members 22052 are held in the first configuration by the first actuation member and/or the second actuation member. Conversely, when the first actuation member and/or the second actuation member (not shown) is moved within the passage 22050 such that the first and/or second actuation member (not shown) is not adjacent the one or more locking members 22052, the locking members 22052 move to their second configuration (e.g., the outwardly extending, locking configuration).

In some implementations, to move the expandable/expansion member 22018 between the first configuration and the second configuration, the distal member 22042 can be moved axially within the passage 22063 of the proximal member 22044.

In some implementations, the distal member 22042 can be moved proximally within the passage 22063 by pulling on the first actuation element (not shown) which is coupled to the proximal end 22046 of the distal member 22042. Since the distal end 22048 of the distal member 22042 is fixed to the distal end portion 22024 of the expandable/expansion member 22018 by the stop 22084 and the end cap 22066 fixes the proximal member 22044 in place relative to the expandable/expansion member 22018, pulling on the first actuation element (not shown) with sufficient force moves the distal end portion 22024 of the expandable/expansion member 22018 closer to the proximal end portion 22022 of the expandable/expansion member 22018. As a result, the strips 22028 of the intermediate portion 22026 bend or flex outward. Since the strips 22028 are attached to the frame member (e.g., frame member 21002), the outward expansion of the strips 22028 causes the outward expansion of the frame member.

In some implementations, once the expandable/expansion member 22018, and thus the expandable coaptation element, has been expanded to the desired width or diameter, the distal member 22042 can be locked in position relative to the proximal member 22044. In particular, the first actuation member and/or second actuation member (not shown) can be moved axially within the passage 22050 such that the first actuation member and/or the optional second actuation member (not shown) is not adjacent the one or more locking members 22052. As a result, one or more locking members 22052 pivot to their second configuration resulting in the first end 22053 of each of the locking members 22052 engaging with one of the slots 22060 in the proximal member 22044. In some implementations, the engagement of the first end 22053 of each of the locking members 22052 with a corresponding slot 22060 locks the distal member 22042 from moving axially within the passage 22063 in the proximal direction.

In some implementations, the frame member (e.g., frame member 21002) and/or the expandable/expansion member 22018 can be configured to be normally in the first configuration (i.e., collapsed or narrow), in the second configuration (i.e., expanded or wide), or some intermediate position between the first and the second configuration. In some implementations, movement of the distal member 22042 relative to the proximal member 22044 pulls the frame member and/or the expandable/expansion member 22018 inward toward the first position against a bias of the frame member and/or the expandable/expansion member 22018 or, in some implementations, pushes the frame member and/or the expandable/expansion member 22018 outward to the second position against a bias of the frame member and/or the expandable/expansion member 22018.

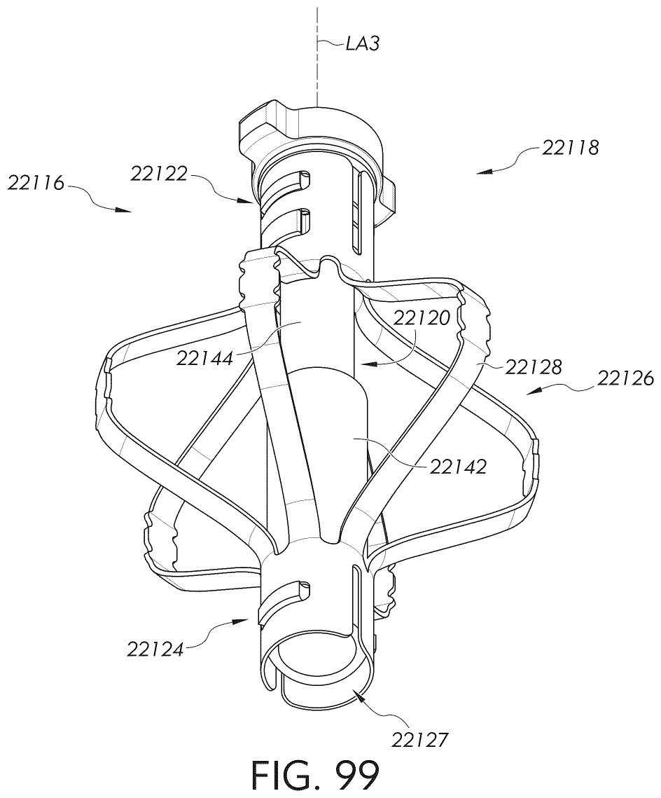

FIG. 99 illustrates an example expandable mechanism 22116 for use in an expandable coaptation element. The expandable mechanism 22116 can be the same as or similar to any of the expandable mechanisms described anywhere in this disclosure. The expandable mechanism can comprise one or more of a balloon, expandable container, expandable material, self-expanding material, self-expanding frame, stent, mechanically-expandable frame, pivoting and/or scissoring extensions and/or struts, expanding pully mechanism, Hoberman mechanism, cam, worm screw, rack and pinion, foam, etc.

In some implementations, the expandable mechanism 22116 can be positioned and/or mounted inside an expandable frame member, such as the frame member 21002 (FIG. 82-83). The example expandable mechanism 22116 includes an expandable/expansion member 22118 and an actuation mechanism 22120. The expandable/expansion member 22118 can be configured in a variety of ways, e.g., the same as or similar to any other expandable/expansion members herein. The actuation mechanism 22120 can be configured in a variety of ways, e.g., the same as or similar to any other actuation mechanisms herein.

In the illustrated example, the expandable/expansion member 22118 is substantially similar to the expandable/expansion member 21018 of FIGS. 87-88, thus the description of the expandable/expansion member 21018 applies equally to the expandable/expansion member 22118. In particular, the expandable/expansion member 22118 is configured as an elongated cylindrical shape having a proximal end portion 22122, a distal end portion 22124, an intermediate portion 22126, and a passage 22127 extending through the expandable/expansion member 22118 from the proximal end portion 22122 to the distal end portion 22124. In some implementations, the intermediate portion 22126 is formed from a plurality of strips 22128 that are configured to bend or flex.

In some implementations, the expandable/expansion member 22118 is movable between a first configuration (i.e., collapsed or narrow configuration) (e.g., FIG. 88 for expandable/expansion member 21018) and a second configuration (i.e., expanded or wide configuration), as shown in FIG. 99. In some implementations, in the first configuration, each of the plurality of strips 22128 extend parallel to a longitudinal axis LA3 of the expandable/expansion member 22118 and, in the second configuration, the strips 22128 bend or flex outward such that the intermediate portion 22126 expands radially outward.

The actuation mechanism 22120 can be configured in a variety of ways, e.g., in any of the ways other actuation mechanisms are configured herein. In the illustrated example, the actuation mechanism 22120 includes a distal member 22142 and a proximal member 22144. In some implementations, the distal member 22142 and proximal member 22144 are arranged coaxially in a telescoping fashion such that a portion of the proximal member 22144 is received within the distal member 22142 and can move axially relative to the distal member 22142 or vice versa. In some implementations, the movement of the distal member 22142 and the proximal member 22144 axially relative to each other moves the expandable/expansion member 22118 between a first configuration and a second configuration. The distal member 22142 and the proximal member 22144 are movable relative to each other by any suitable means, such as for example, any of the actuation mechanisms disclosed herein.

FIGS. 100-101 illustrate an example expandable coaptation element 21000 as part of a device (e.g., valve repair device, valve treatment device, implantable device, implant, etc.) that is configured to be positioned within a native heart valve to allow the native heart valve to form a more effective seal. The expandable coaptation element 21000 is shown attached to gripping members (e.g., gripping arms, clasp arms, etc.), such as for example, gripping members 130.

In some implementations, the gripping members 130 can be used to attach the expandable coaptation element 21000 to native leaflets of a native heart valve. For example, the gripping members 130 can be opened to accept leaflets of a native mitral valve or a native tricuspid valve and then closed to capture the leaflets and thereby attach the expandable coaptation element 21000 to the native heart valve.

In some implementations, the expandable coaptation element 21000 includes the frame member 21002 and the expandable/expansion member 21018. FIG. 100 illustrates the expandable coaptation element 21000 in a first configuration (i.e., collapsed or narrow) and FIG. 101 illustrates the expandable coaptation element 21000 in a second configuration (i.e., expanded or wide). The expandable coaptation element 21000 can be moved between the first configuration and the second configuration by any suitable means, such as for example, any of the actuation mechanisms disclosed herein.

In some implementations, the expandable coaptation element 21000 can include an optional cover over the frame member 21002. The cover can cover a portion or all of the frame member 21002.

Referring to FIGS. 102-110, an example of a cover 102951 for the expandable coaptation element 21000 is shown. The cover 102951 can be used with any suitable expandable coaptation element 21000, such as, for example, any expandable coaptation element 21000 described in the present application. In some implementations, the cover 102951 is configured to be attached to frame member 21002. However, it should be understood that the cover 102951 can be configured for connecting to any component of the device.

The cover 102951 can be configured in a variety of ways. In some implementations, the cover 102951 can include a sheet, material, fabric, layer or membrane that is attached to the frame member 21002 by a plurality of connectors (e.g., stitches, adhesive, mechanical fasteners, ultrasonic welds, etc.). In some implementations, the sheet, material, fabric, layer or membrane can be made of a flexible material, a porous material, and/or a material that is impermeable to blood flow. In some implementations, the sheet, material, fabric, layer, or membrane is made from a biocompatible material, such as a woven biocompatible fabric that is configured to promote tissue ingrowth. The cover 102951 can be configured to cover or not cover any component or portion of the device.

In some implementations, the cover 102951 can have one or more stretchable portions 102953 that allow the cover 102951 to maintain a substantially taut state when in a normal position, while also allowing the cover 102951 to stretch to an expanded state. This is advantageous for situations in which the cover 102951 is attached to components of the device, such as the expandable coaptation element 21000, that are movable between narrowed and expanded positions. That is, the cover 102951 maintaining a taut state when in a normal position, and when expandable coaptation element 21000 are in a narrowed position, reduces any excess material on the device that can contact vasculature.

In some implementations, the stretchability of the cover 102951 then allows the expandable coaptation element 21000 to move to an expanded state with the cover 102951 maintaining a substantially taut state and maintaining a covering for the device. The stretchable portions 102953 can take a wide variety of different forms. Any material that can stretch and return to its original size or substantially to its original size can be used.

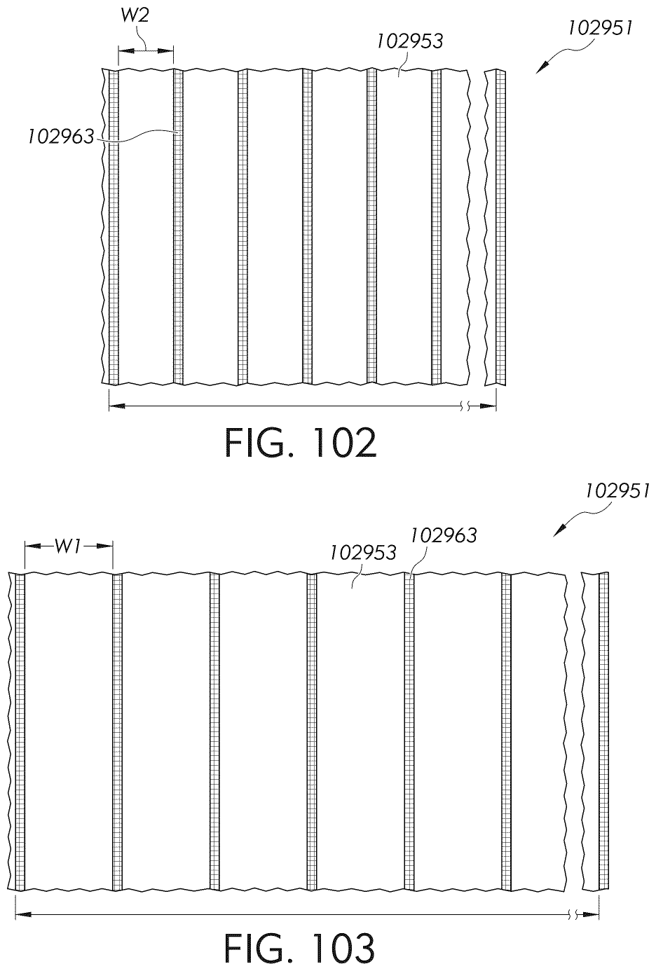

Referring to FIGS. 102-103, a portion of an example cover 102951 is shown as a flat sheet of material or formed as a flat sheet. In some implementations, the cover 102951 includes different shaped segments or portions to attach to different portions of the frame member 21002. In some implementations, the cover 102951 can be shaped to smooth transitions between various portions of the device to reduce catch points and provide a smoother exterior to the device.

In the illustrated example, the cover 102951 includes a plurality of plain weave portions 102963 spaced apart and connected by stretchable portions 102953. In some implementations, the plain weave portions 102963 are configured to attach to the frame member 21002. For example, each of the plain weave portions 102963 can attach to a corresponding post 21012 of the frame member 21002.

In some implementations, when the frame member 21002 expands from the first configuration (i.e., collapsed or narrow), as shown in FIG. 102, to the second configuration (i.e., expanded or wide), as shown in FIG. 103, the stretchable portions 102953 stretch laterally from a width W2 to a width W1, which is larger than the width W2 to accommodate the expansion of the frame member 21002.

Referring to FIG. 105, the plain weave portions 102963 can include weft yarns 102965 and warp yarns 102967 that are woven in a perpendicular weaving pattern. However, the plain weave portions 102963 can take any other suitable form, or the cover 102951 can include any other type of main weaving pattern with the stretchable portions 102953 positioned therein. In some implementations, the plain weave portions 102963 is made of a woven biocompatible fabric that is configured to promote tissue ingrowth. The plain weave portions 102963 can also be configured to reduce blood regurgitation.

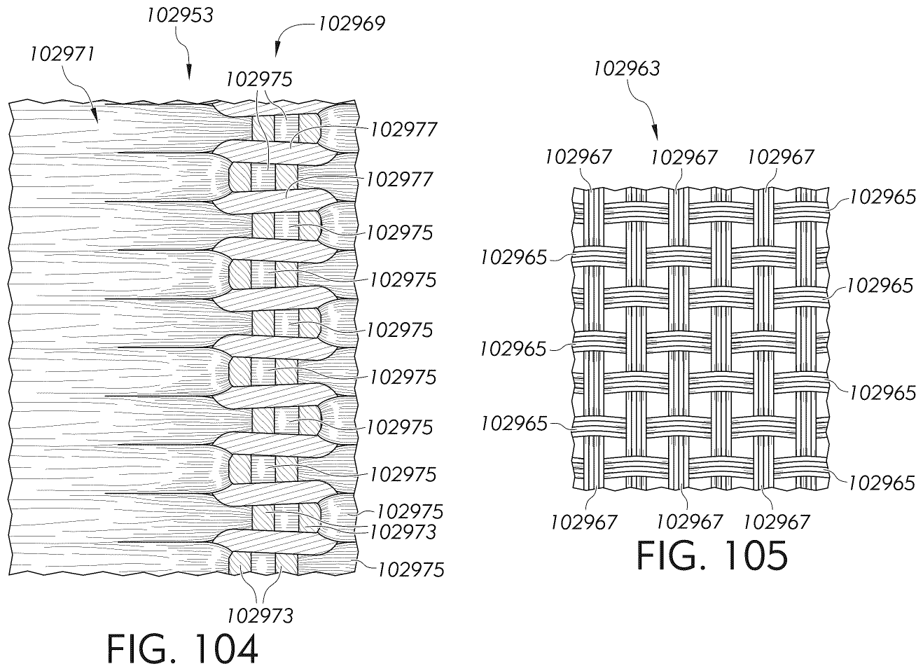

Referring to FIGS. 104 and 106-107, in some implementations, the stretchable portions 102953 can include a pair of transition portions 102969, such as Leno weaves, with a stretchable material or float 102971 connected therebetween. In some implementations, the transition portion, such as a Leno weave, provides a transition, connection or interface between the plain weave 102963 and the stretchable material or float.

Referring to FIG. 104, the float 102971 can include a portion of threads that are not interlaced with any other threads. In some implementations, the floats 102971 include textured yarn. However, other configurations are contemplated.

In some implementations, the Leno weaves 102969 can include warp yarns 102973 and weft yarns 102975 that are woven in a perpendicular weaving pattern with at least one Leno yarn 102977 wrapping the warp yarns 102973.

In the illustrated example, a single Leno yarn 102977 wraps around four warp yarns 102973, and the weft yarns 102975 are extensions of the yarns from the float 102971. In some implementations, the weft yarns 102975 from the Leno weave 102969 extends to the weft yarns 102965 of the plain weave 102963 (FIG. 105). In some implementations, the plain weave 102963 is separate from the Leno weave 102969, and the weft yarns 102975 of the Leno weave 102969 are folded back over or otherwise connected to the warp yarns 102973 and/or Leno yarn 102977 of the Leno weave 102969. However, other configurations for the stretchable portions 102953 are contemplated. The Leno weave 102969 produces an open fabric that prevents or inhibits slippage or misplacement of threads. The Leno weave 102969 can provide further resistance to blood regurgitation.

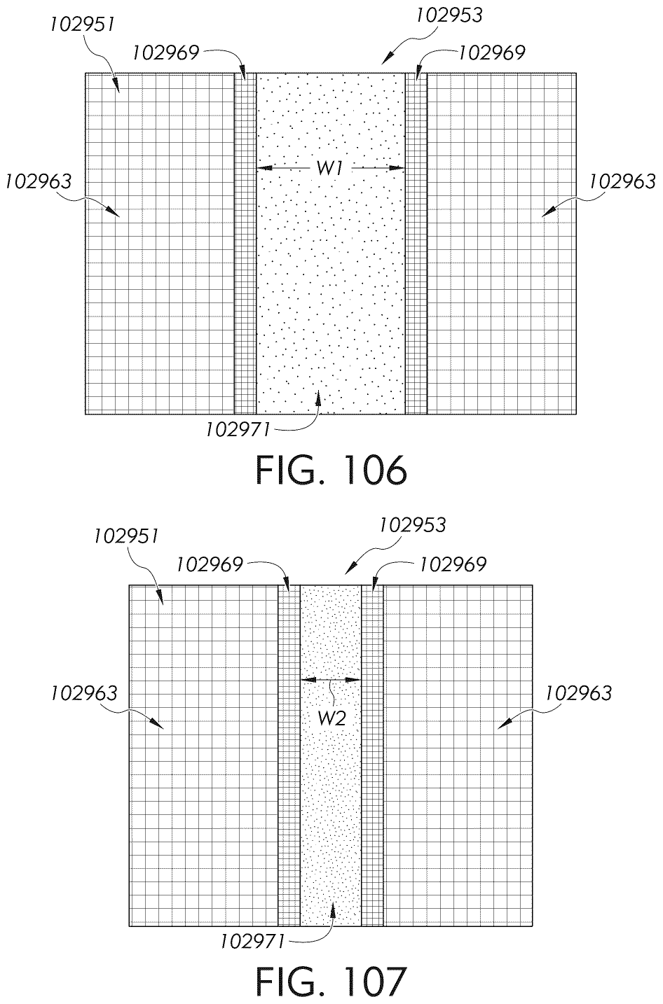

Referring to FIGS. 106-107, in some implementations, the stretchable portions 102953 can be created by taking a cover 102951 that includes the plain weave 102963, two or more Leno weaves 102969, and one or more floats 102971 described above in an initial state (as shown in FIG. 106) and heating the cover 102951 such that the floats 102971 shrink to a narrowed state (as shown in FIG. 107). For example, the yarn of the floats 102971 can crimp and/or curl during heating and cause the floats 102971 to move to the narrowed state. Heat setting or heat pressing the cover 102951 can cause the floats 102971 to shrink to the narrowed state.

In some implementations, once the floats 102971 shrink to the narrowed state, the cover 102951 normally has a width that is based on the floats being in the narrowed state, but the floats can be pulled to expand to a stretched state such that allows the width of the cover 102951 to expand when tension is applied to the cover. For example, pulling the stretchable material or float can temporarily straighten out the curled and/or kinked strands of the stretchable material or float.

In some implementations, removal of the tension from the cover 102951 causes the floats to move back to the narrowed state such that the cover 102951 moves back to the normal position. That is, the strands pull back to the curled and/or kinked heat set configuration.

Heating the cover 102951 can also provide advantages to the plain weave 102963. For example, heat setting or heat pressing the plain weave 102963 can reduce the pore size and/or increase the density of the plain weave 102963, which can be advantageous in preventing or inhibiting blood regurgitation.

In some implementations, the floats 102971 can have a width W1 (FIG. 106) when in the pre-heated state and a width W2 (FIG. 107) after being heated. In some implementations, the width W1 can be between about 3 mm and about 7 mm or any subrange, and the width W2 can be between about 1 mm and about 3 mm or any subrange. The ratio of the width W1 to the width W2 can be between about 1.1 to 1 and about 7 to 1 or any subrange. However, the width W1 and the width W2 can take any other suitable sizes based on the portion of the device that the cover 102951 is connected to or the desired amount of stretch for the cover 102951.

In some implementations, the cover 102951 is configured to maintain a substantially taut state when in a normal position, while also allowing the cover 102951 to stretch to an expanded state, but the cover 102951 does not include the discrete stretchable portions 102953.

The cover 102951 can be configured to be stretchable and/or resilient in a variety of different ways. In some implementations, the cover 102951 is made to be stretchable by rotating the material of the cover such that the horizontal and vertical yarns of the weave are no longer horizontal and vertical before the cover is cut from the material. For example, the material that forms the cover can be rotated between 30 degrees and 60 degrees, such as between 40 and 50 degrees, such as about 45 degrees or 45 degrees. The rotation of the fabric that forms the material of the cover allows the cover to stretch as the frame member is moved between the narrow and wide configurations. It should be understood, however, that the cover 102951 can be configured to stretch in a wide variety of different ways.

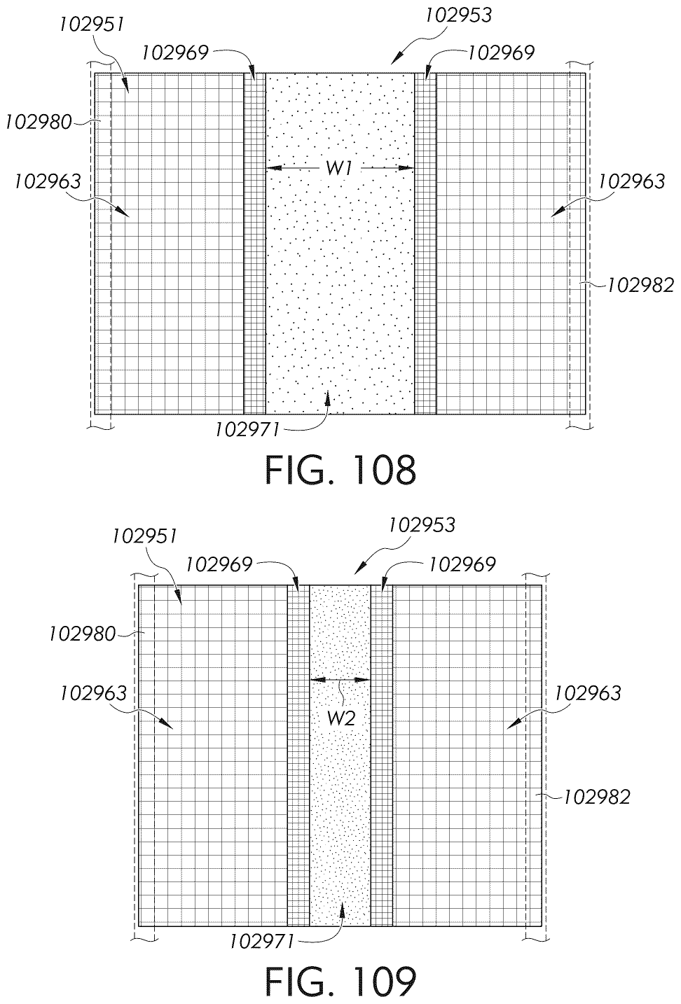

Referring to FIGS. 108-109, in some implementations, the cover 102951 can be attached to a first post 102980 and a second post 102982 of a frame member for an expandable coaptation device (e.g., frame member 21002 of FIG. 82). In the illustrated example, the cover 102951 includes a single stretchable portion 102953 that takes the form of the stretchable portion shown in FIGS. 106-107. However, the cover 102951 can have any suitable number of stretchable portions 102953 between the first and second posts 102980, 102982. Referring to FIG. 108, when the posts 102980, 102982 are in an expanded state, the stretchable portion 102953 can stretch such that the float 102971 has a width W1, which can be substantially identical to or less than the width W1 of the pre-heated float 102971 shown in FIG. 106. Referring to FIG. 109, when the posts 102980, 102982 are in a narrowed state, the stretchable portion 102953 move back to the normal position such that the float 102971 has a width W2, which can be substantially identical to or greater than the width W2 of the heat set float 102971 shown in FIG. 107.

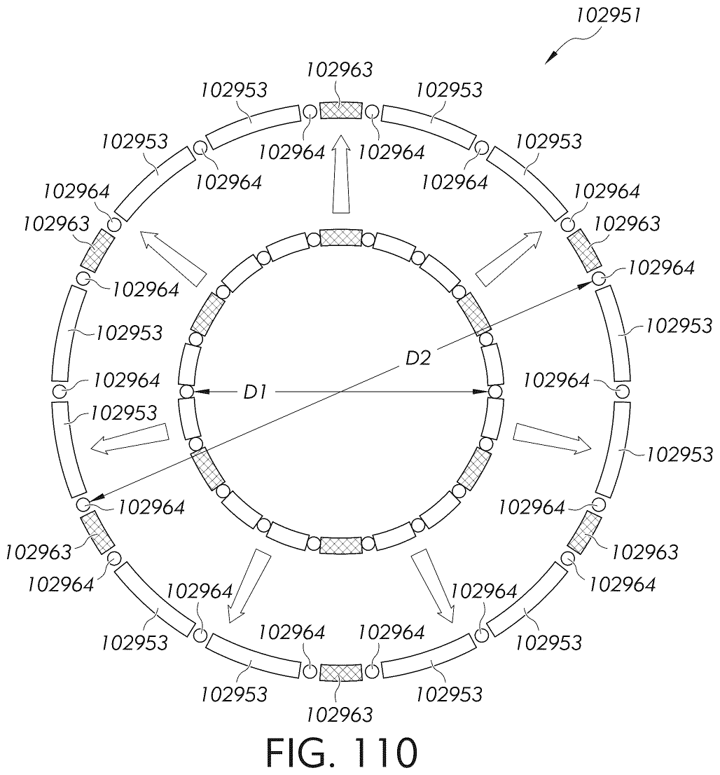

FIG. 110 shows a schematic top view of an example cover 102951 for an expandable coaptation element in both the narrowed state and the expanded state. In some implementations, the cover 102951 includes a plurality of plain weave portions 102963 spaced apart and connected to one or more stretchable portions 102953. In the illustrated example, the cover 102951 includes six plain weave portions 102963 evenly spaced apart. In some implementations, however, the cover 102951 can include more or less than six plain weave portions 102963 and/or the plain weave portions 102963 can be unevenly spaced apart. In some implementations, two or more plain weave portions 102963 can be connected side-by-side.

In the illustrated example, each plain weave portions 102963 is connected to another plain weave portion 102963 by a pair of stretchable portions 102953. In some implementations, however, the cover 102951 can include more or less than two stretchable portions 102953 connecting two plain weave portions 102963. Plain weave portions 102963 can be connected to stretchable portions 102953 or other plain weave portions 102963 and stretchable portions 102953 can be connected to other stretchable portions 102953 in any suitable manner. In the illustrated example, Leno stitches 102964 are used to connect two stretchable portions 102953 together and plain weave portions 102963 to stretchable portions 102953.

As shown in FIG. 110, the cover 102951 can stretch from a first diameter D1 in the first state to a second diameter D2 in the second state. In some implementations, the ratio of D2:D1 is in the range to 1.5:1 to 3:1. As shown in FIG. 110, the stretchable portions 102953 account for the majority of the increase in diameter of the cover 102951 when expanded. For example, in some implementations, the plain weave portion 102963 expand in length less than 5%, less than 4%, less than 3% or less than 2% when the cover expands from the first state to the second state.

Any of the covers disclosed herein, such as the cover 102951 illustrated by FIG. 110 can be formed in a variety of different ways. In some implementations the cover is woven as a tube. For example, the material of the cover 102951 can be woven as a tube around a mandrel and/or by a circular weaving machine.

In some implementations, the cover 102951 can be woven as a flat sheet. Cover material woven as a flat sheet can be formed into a tube or another shape, for example, by connecting two opposed ends of the flat sheet together (e.g., with stitches).

Any of the covers disclosed herein can be woven as a tube, woven as a flat sheet, woven in some other shape/configured, and/or some portion(s) of the cover can be woven as a tube, while other portion(s) of the cover can be woven as a sheet, etc.

In some implementations, a cover, such as any of the covers described herein can be configured and/or treated to reduce blood flow through the cover. In some implementations, covers configured and/or treated to reduce blood flow through the cover can be expandable/stretchable or non-expandable/non-stretchable.

The cover can be configured and/or treated to reduce blood flow through the cover in a variety of different ways. For example, the cover can be configured and/or treated to reduce blood flow through the cover by reducing pore size. The pore size can be reduced in a variety of different ways. For example, the pore size can be reduced by increasing the density of the fabric, by laminating the fabric, by coating the fabric, by layering the fabric, and/or by other means.

The density of the fabric can be increased in a variety of different ways. In some implementations, the ends per inch (EPI) and/or the picks per inch (PPI) of one or more portions of the fabric can be increased to increase the density of the fabric and reduce the permeability of the fabric. For example, the EPI X PPI can be between 160×152 and 400×500 or any sub range. The EPI×PPI for a polyethylene terephthalate (PET) yarn can be between 160×152 and 400×500 or any sub range. The EPI×PPI for a 50% polyethylene terephthalate (PET) and 50% polyolefin (PO) yarn can be between 160×152 and 400×500 or any sub range. The EPI X PPI can be 160×196. The EPI×PPI can be 160×256. The EPI×PPI can be 160×304. The EPI×PPI can be 160×152. The EPI×PPI can be 160×196 for a PET yarn. The EPI X PPI can be 160×256 for a PET yarn. The EPI×PPI can be 160×304 for a PET yarn. The EPI X PPI can be 160×152 for a 50/50 PET and PO yarn.

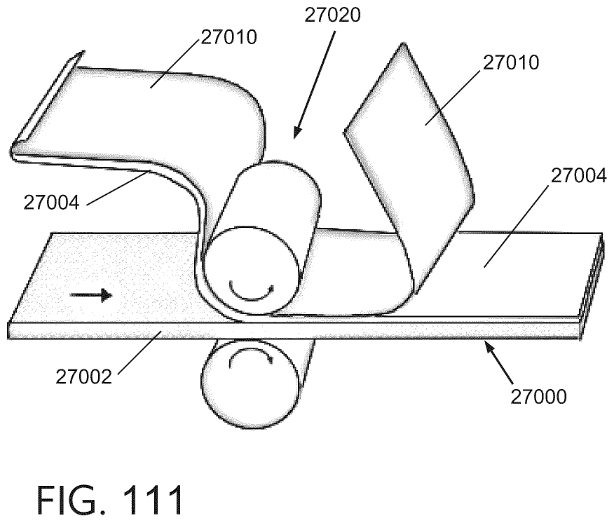

Referring to FIG. 111, in some implementations a cover material 27000 comprises a fabric 27002 that is laminated with a coating 27004 to decrease the permeability of the cover material. The coating 27004 can be a variety of different materials that are configured to reduce the permeability of the cover material 27000. For example, the coating 27004 can be a silicone, TPU, polyolefin and/or other polymer-based coating.

The fabric 27002 can take a variety of different forms. For example, the fabric 27002 can be any of the expandable and/or non-expandable fabrics disclosed herein.

In the example illustrated by FIG. 111 a release paper 27010 is coated with the coating 27004. In some implementations, the coated release paper 27010 and the fabric 27002 are passed through a pair of nip rollers 27020. The nip rollers 27020 press and transfer the coating from the release paper 27010 to the fabric 27002. In some implementations, the release paper 27010 is separated from the fabric 27002, leaving the coated cover material 27000 with a reduced permeability.

In some implementations, a yarn that reduces permeability can be woven into a fabric of the cover material. For example, a TPU, silicone, polyolefin and/or elastic yarn can be woven into any of the cover materials disclosed by the present application.

In some implementations a cover material comprises a fabric that is dip coated to decrease the permeability of the cover material. The coating can be a variety of different materials that are configured to reduce the permeability of the cover material. For example, the dip coating can be a silicone, TPU, polyolefin and/or other polymer-based coating. The fabric can take a variety of different forms. For example, the fabric can be any of the expandable and/or non-expandable fabrics disclosed herein.

In some implementations, the fabric of the cover material is optionally dip coated by first dipping the fabric in acetone. Then, the fabric is optionally dipped in water or distilled water. Then the fabric is dipped in a coating, such as polyurethane or TPU or another polymer coating material. Then, excess coating is optionally removed from the fabric. For example, the fabric can optionally be passed through a nip roller to press out the excess coating. Then, the coated fabric can optionally be dried.

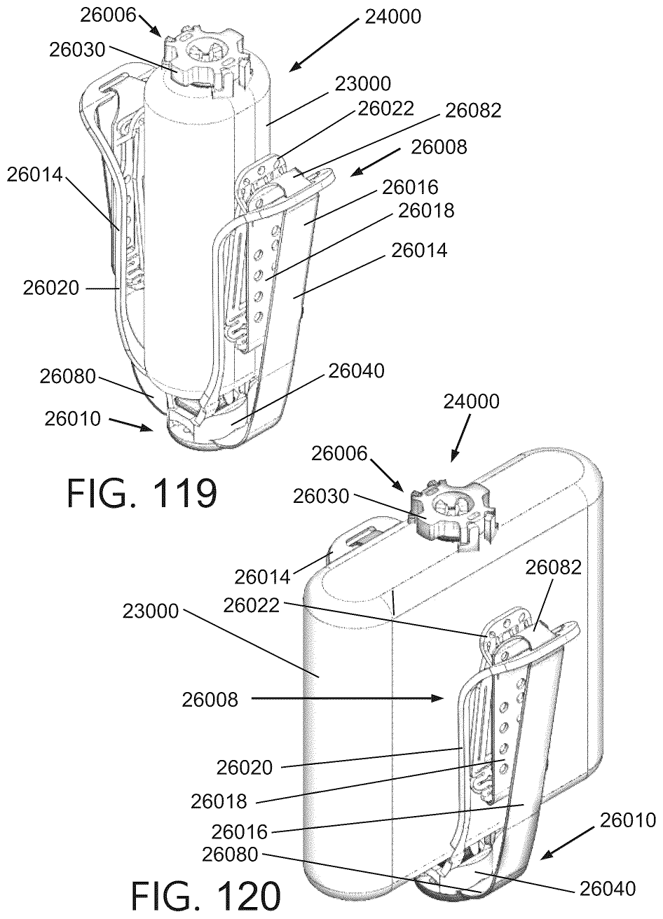

FIG. 113 illustrates an example frame member 23002 (e.g., frame, body, cage, fixture, chassis, form, etc.) for an expandable coaptation element 23000 (e.g., FIG. 119-121). The expandable coaptation element 23000 (e.g., spacer, coaption element, gap filler, membrane, sheet, plug, wedge, balloon, etc.) can be part of a device 24000 (e.g., a treatment device, a repair device, etc.) for repairing a native heart valve and can be used as part of any device described herein.

In some implementations, the frame member 23002 is configured to move between a first contracted configuration (see FIG. 119) and a second expanded configuration (see FIG. 120). The frame member 23002 can be configured in a variety of ways. In some implementations, the frame member 23002 has a generally cylindrical shape (i.e., a circular cross section) in the contracted condition and an oval, elliptical, oblong, etc. shape (i.e., oval, elliptical, oblong, etc. cross section) in the expanded configuration. In some implementations, however, the frame member 23002 can have other shapes in the contracted and/or expanded configurations.

In some implementations, the frame member 23002 can include a plurality of interconnected struts 23008 that are configured to flex or bend to allow the frame member 23002 to move between the first and second configurations. The interconnected struts 23008 can be configured in a variety of ways, such as for example, the number and size of the struts, the shape of each strut, the arrangement of struts relative to other struts, the interconnection of the struts, etc. Any configuration that can facilitate the expansion and contraction of the frame member 23002 can be used. In some implementations, the struts 23008 are arranged in diamond patterns.

In some implementations, the example frame member 23002 includes a plurality of posts 23012 interconnected by the plurality of struts 23008. The posts 23012 can be configured in a variety of ways, such as for example, the number and size of the posts, the shape of each post, the arrangement and connection of posts relative to the struts, etc. In the illustrated example, each post 23012 extends linearly from a proximal end 23004 to a distal end 23006. In the illustrated example, each post 23012 is connected on either side, along a midpoint 23014, to one of the diamond patterns 23010 of struts 23008.

In some implementations, the frame member 23002 is formed as a unitary piece. For example, the frame member 23002 can be laser-cut from a tube. In some implementations, however, some of the struts 23008 and the posts 23012 can be formed separately and can be connected in any suitable manner (e.g., welded together).

In some implementations, the frame member 23002 can comprise a flexible material that can be a metal fabric, such as a mesh, woven, braided, or formed in any other suitable way or a laser cut or otherwise cut flexible material. In some implementations, the material can comprise cloth, shape-memory alloy wire—such as Nitinol—to provide shape-setting capability, or any other flexible material suitable for implantation in the human body.

In some implementations, when the frame member 23002 expands, the height of each of the diamond patterns 23010 decreases while the width increases. Conversely, the height of each post 23012 does not change. Thus, the frame member 23002 has a fixed height (i.e., the height of the posts) that does not change between the first and second configurations.

In some implementations, the frame member 23002 can be configured to change height between the first and the second configuration. For example, the posts can be replaced with diamond patterns, such that the entire frame member 23002 decreases in height as the frame member increases in width.

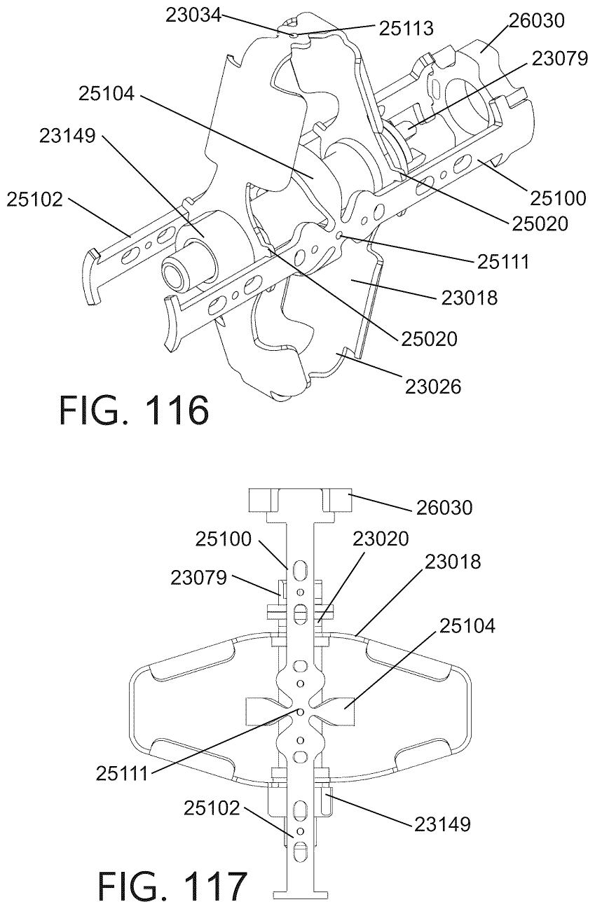

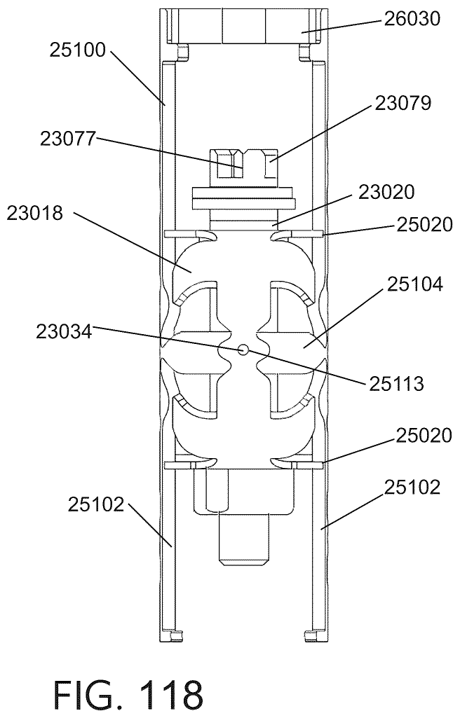

The frame member 23002 can be moved between the first and the second configurations by any suitable means. For example, in some implementations, the frame member 23002 can be mounted around an expandable mechanism 23016 (see FIGS. 116-118) configured to engage the frame member 23002 to move the frame member 23002 between the first and the second configurations. A variety of different mechanisms can be used to move the frame member 23002 between the first and second configurations. For example, any expandable mechanism disclosed herein can be used.

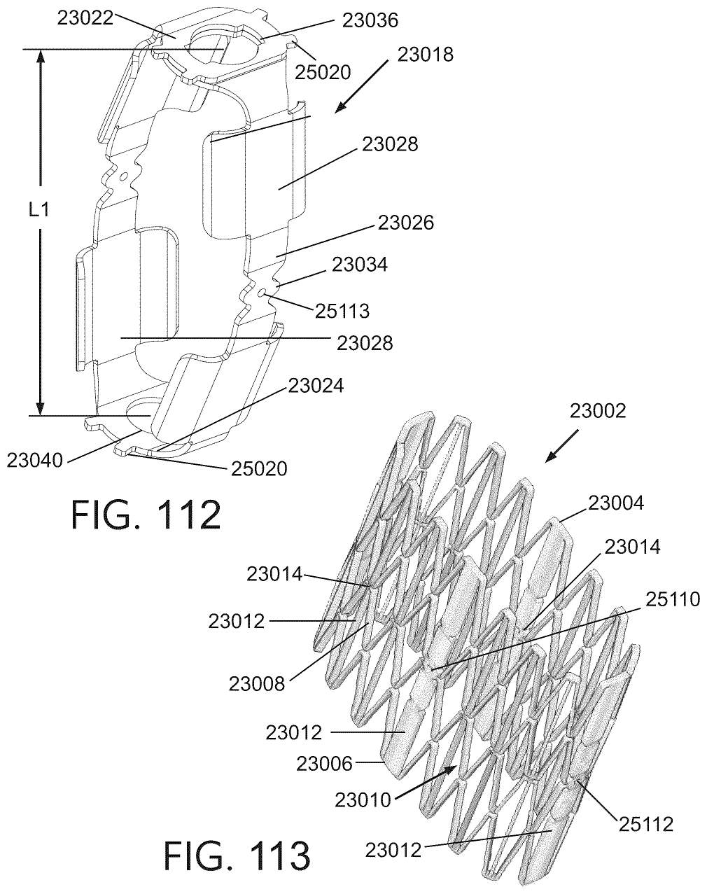

In some implementations, as shown in FIGS. 112 and 114-118, the expandable mechanism 23016 includes an expandable or expansion member 23018 (e.g., frame, body, strut assembly, tube, shaft, articulating member, foldable component, balloon, cam, etc.) and an actuation mechanism 23020. In some implementations, the frame member 23002 is mounted around the expandable/expansion member 23018.

The expandable or expansion member 23018 can be configured in a variety of ways. Any configuration capable of moving the frame member 23002 between the first configuration and the second configuration can be used.

Referring to FIG. 112, in some implementations, the expandable or expansion member 23018 is configured as an elongated shape having a proximal end portion 23022, a distal end portion 23024 opposite the proximal end portion 23022, and an intermediate portion 23026 connecting the proximal end portion 23022 and the distal end portion 23024. In some implementations, the intermediate portion 23026 is formed from a pair of strips 23028 that are configured to bend or flex.

In some implementations, the expandable/expansion member 23018 includes two opposed strips 23028. In other implementations, however, the strips 23028 may not be equally spaced and/or the intermediate portion 23026 can include more or less than two (2) strips.

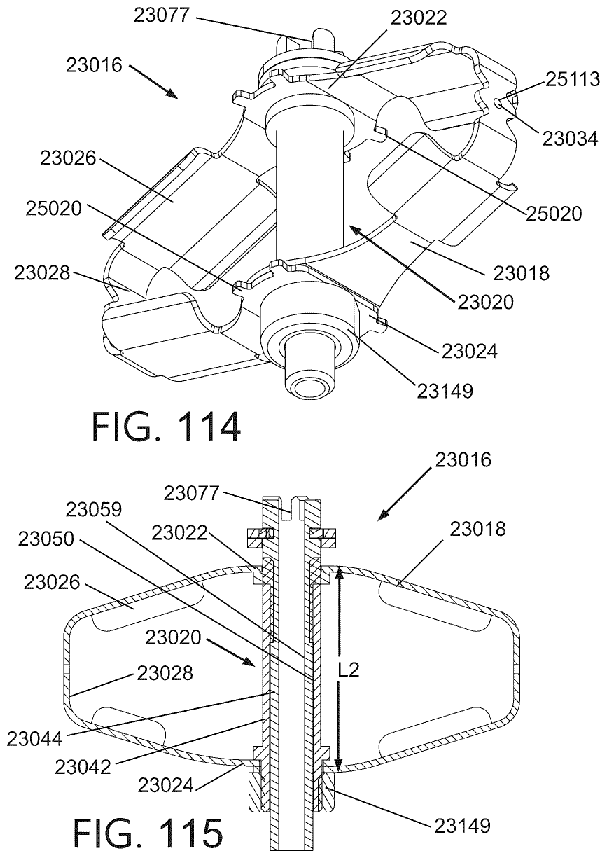

In some implementations, the expandable/expansion member 23018 is movable between a first configuration (e.g., unexpanded, collapsed, and/or narrow configuration) (FIG. 112) and a second configuration (e.g., an expanded or wide configuration) (FIG. 114). As shown in FIG. 112, in the first configuration, each of the plurality of strips 23028 extend parallel or generally parallel to a longitudinal axis of the expandable/expansion member 23018 and the expandable/expansion member 23018 has a first length L1.

In some implementations, in the first configuration, the proximal end portion 23022, the distal end portion 23024, and the intermediate portion 23026 have the same or substantially the same width.

In some implementations, the expandable/expansion member 23018 can be formed as a unitary piece. For example, the expandable/expansion member 23018 can be laser-cut from a single piece of sheet material. In some implementations, the expandable/expansion member 23018 can be formed from multiple pieces, such as for example, a pair of Nitinol sheets.

In some implementations, the expandable/expansion member 23018 can comprise a flexible material that can be a metal fabric, such as a mesh, a woven material, a braided material, or a material formed in any other suitable way or a laser cut or otherwise cut flexible material. The material can be cloth, shape-memory alloy wire—such as Nitinol—to provide shape-setting capability, or any other flexible material suitable for implantation in the human body.

In some implementations, as shown in FIG. 114, in the second configuration, the proximal end portion 23022 is moved toward the distal end portion 23024, or vice versa, or the proximal end portion and the distal end portion simultaneously move toward one another, causing the strips 23028 to bend or flex outward such that the intermediate portion 23026 expands to a second width that is greater than the first width.

In some implementations, in the second configuration, the expandable/expansion member 23018 has a second length L2, that is less than the first length L1 and the width of the proximal end portion 23022 and the distal end portion 23024 remain at the first width while the intermediate portion 23026 expands to the second width.

In some implementations, the expandable/expansion member 23018 positioned within the periphery of the frame member 23002 to engage the frame member 23002. In some implementations, a midpoint 23034 of each of the two strips 23028 is attached to the frame member 23002. In some implementations, the midpoint 23034 of each strip 23028 is attached to one of the posts 23012 of the frame member 23002.

In some implementations, the midpoint 23034 of each strip 23028 can be attached to the frame member 23002 in any suitable manner, such as for example, welding, sutures, fasteners, rivets, etc.

In some implementations, as a result of the expandable/expansion member 23018 being positioned within the periphery of the frame member 23002 and attached to the frame member 23002, movement of the expandable/expansion member 23018 between the first configuration (e.g., unexpanded, collapsed, and/or narrow configuration) (FIG. 112) and a second configuration (e.g., expanded and/or wide configuration) (FIG. 114) moves the frame member 23002 between the frame member's first configuration (e.g., unexpanded or collapsed-FIG. 119) and second configuration (e.g., expanded-FIG. 120).

In some implementations, the frame member 23002 and/or the expandable/expansion member 23018 can be configured to be normally in the first configuration (e.g., unexpanded, collapsed, and/or narrow), in the second configuration (e.g., expanded, wide, etc.), or some intermediate position between the first configuration and the second configuration.

In some implementations, movement of the expandable/expansion member 23018 pulls the frame member 23002 inward toward the first position against a bias of the frame member 23002. In some implementations, movement of the expandable/expansion member 23018 pushes the frame member outward to the second position against a bias of the frame member 23002.

Referring to FIGS. 114-115, in some implementations, an actuation mechanism 23020 is configured to move the expandable/expansion member 23018 between the first and the second configurations. The actuation mechanism 23020 can be configured in a variety of ways. Any actuation mechanism capable of moving the expandable/expansion member 23018 between the first and the second configurations can be used, e.g., the actuation mechanism can comprise one or more of an actuation wire, actuation element, pivoting link, cam, rack and pinion, worm screw, lever, pulley, articulating arm, etc. In some implementations, the actuation mechanism 23020 is mounted within the expandable/expansion member 23018.

Referring to FIGS. 114-118, in some implementations, the expandable/expansion member 23018 can include structure configured to mount the actuation mechanism 23020 within the expandable/expansion member 23018. The structure can be configured in a variety of ways. In some implementations, the proximal end portion 23022 of the expandable/expansion member 23018 includes a first opening 23036 and the distal end portion 23024 of the expandable/expansion member 23018 includes a second opening 23040.

In some implementations, the actuation mechanism 23020 is configured as a threaded connection. Referring to FIGS. 114-115, the example actuation mechanism 23020 includes a distal member or outer member 23042 (e.g., tube, body, shaft, etc.) configured to threadably couple with a proximal member or inner member 23044 (e.g., tube, body, shaft, etc.).

The proximal or inner member 23044 and the distal or outer member 23042 can be configured in a variety of ways. In the illustrated example, the distal member 23042 is formed as a cylindrical tube having a circular cross section. In some implementations, however, the distal member 23042 can have a shape other than cylindrical (e.g., a cross section that is oval, rectangular, elliptical, or other suitable shape).

In some implementations, the distal member 23042 includes a proximal end, a distal end opposite the proximal end, and an inner passage 23050 extending through the distal member 23042 member from the proximal end to the distal end. In some implementations, the inner passage 23050 can include female threads for threadably coupling with the proximal member.

In some implementations, the distal member 23042 is sized to be received within the expandable/expansion member 23018. In some implementations, the assembly includes structure for fixing the distal member 23042 in position within the expandable/expansion member 23018. In the illustrated example, a nut or collar 23149 fixes the distal end of the distal member 23042 to the distal end portion 23024 of the expandable/expansion member 23018, as shown in FIGS. 114-115.

In some implementations, the proximal member 23044 is formed as a cylindrical tube having a circular cross section. In some implementations, the proximal member 23044 can have a shape other than cylindrical (e.g., a cross section that is oval, rectangular, elliptical, conical, or other suitable shape). In some implementations, the proximal member 23044 includes a proximal end, a distal end opposite the proximal end, and an inner passage 23059 extending through the proximal member 23044 from the proximal end to the distal end. In some implementations, the inner passage is sized to accommodate an actuation element (e.g., actuation element 112 for opening and closing paddles of the valve repair device).

In some implementations, the proximal member 23044 can include male threads extending along at least a portion of the exterior of the proximal member 23044. In some implementations, the male threads are configured to mate with the female threads of the distal member 23042. In some implementations, the proximal member 23044 is sized to be received within the expandable/expansion member 23018. In some implementations, the actuation mechanism 23020 can include structure for restricting axial movement of the proximal member 23044 relative to the expandable/expansion member 23018.

In some implementations, the structure restricts axial movement of the proximal member 23044 within the expandable/expansion member 23018 while allowing rotational movement of the proximal member 23044 relative to the expandable/expansion member 23018.

The structure restricting axial movement of the proximal member 23044 can be configured in a variety of ways. For example, a fastener, such as a clip, pin, rivet, etc. can be used to restrict axial movement of the proximal member 23044 within the expandable/expansion member 23018 while allowing rotational movement of the proximal member 23044 relative to the expandable/expansion member 23018.

Referring to FIGS. 112, 114, and 115, in some implementations, when assembled, the actuation mechanism 23020 can move the expandable/expansion member 23018 between the first and the second configurations. In particular, the distal member 23042 is received within the expandable/expansion member 23018. In some implementations, the distal member 23042 can be both axially and rotationally fixed to the expandable/expansion member 23018.

In the example illustrated by FIGS. 114 and 115, the expandable mechanism 23016 is configured to move in one plane (i.e., the two strips 23028 move apart). In some implementations, the expandable mechanism can move in two planes. For example, a second pair of strips can be disposed orthogonally to the first pair of strips.

In some implementations, the second pair of strips can move in a second plane that is orthogonal to the first plane. However, the second plane can be at any angle to the first plane. As such, the frame or spacer 23002 can be expanded in more than one direction.

In some implementations, the expandable mechanism can be configured such that expansion in the first plane can be controlled independently of the expansion in the second plane (e.g., the width of the two pairs of strips can be independently controllable).

In some implementations, the proximal member 23044 is received in the passage 23050 of the distal member 23042 such that the male threads of the proximal member 23044 threadably engage the female threads of the distal member 23042. In some implementations, a rotational device or tool (not shown) can engage the one or more engagement surfaces 23077 to rotate the proximal member 23044 in a first rotational direction (i.e., to thread the proximal member 23044 into the distal member 23042).

In some implementations, with the proximal member 23044 axially fixed to the proximal end portion 23022 of the expandable/expansion member 23018 and the distal member 23042 axially fixed to the distal end portion 23024 of the expandable/expansion member 23018, threading the proximal member 23044 into the distal member 23042 draws the proximal end portion 23022 of the expandable/expansion member 23018 toward the distal end portion 23024. As a result, the strips 23028 bend or flex outward.

In some implementations, when the midpoint 23034 of the strips 23028 is attached to the posts 23012 of the frame member 23002, the outward expansion of the strips 23028 causes the outward expansion of the frame member 23002.

Conversely, in some implementations, rotating the proximal member 23044 in a second rotational direction (i.e., to thread the proximal member 23044 out of the distal member 23042) pushes the proximal end portion 23022 of the expandable/expansion member 23018 away from the distal end portion 23024. As a result, the strips 23028 are pulled inward causing the inward contraction of the frame member 23002. In some implementations, the actuation mechanism 22016 illustrated by FIGS. 94-98 can be used in place of the actuation mechanism 23020.

Referring to FIGS. 116-118, the expandable mechanism 23016 can be assembled with a frame 25100 of the device 24000. The frame 25100 can take a wide variety of different forms. In some implementations, the frame 25100 includes a pair of rails 25102 that are connected together by a central ring 25104.

In some implementations, the proximal end 23022 and/or the distal end 23024 of the expandable/expansion member 23018 are slidably coupled to the rails 25102. The slidable coupling(s) allow proximal end 23022 and/or the distal end 23024 to slide relative to the frame.

The proximal end 23022 and/or the distal end 23024 can be slidably coupled to the rails 25102 in a variety of different ways. Any slidable coupling can be used. In some implementations, the proximal end 23022 includes a pair of tabs 25020 that fit around the rails 25102 to slidably couple the proximal end 23022 to the frame 25100. In some implementations, the distal end 23024 includes a pair of tabs 25020 that fit around the rails 25102 to slidably couple the distal end 23024 to the frame 25100.

In some implementations, the expandable frame member 23002 or spacer is attached to the central ring 25104 and the expandable/expansion member 23018. Since the central ring 25104 is fixed in size, the length (when viewed from an end) of the expandable frame member 23002 or spacer is fixed by the connection to the ring. Since the expandable/expansion member 23018 is adjustable in size, the width of the expandable frame member 23002 or spacer is adjustable by adjusting the width of the expandable/expansion member 23018.

The expandable frame member 23002 or spacer can be attached to the central ring 25104 and the expandable/expansion member 23018 in a variety of different ways. In some implementations, the expandable frame member 23002 or spacer can include two opposed attachment points 25110, such as holes, in two of the posts 23012 for connection to attachment points 25111 of the ring 25104. In some implementations, the expandable frame member 23002 or spacer can include two opposed attachment points 25112, such as holes, in two of the posts 23012 for connection of attachment points 25113 of the expandable/expansion member 23018.

In some implementations, the attachment of the expandable frame member 23002 or spacer to both the ring 25104 and the expandable/expansion member 23018 keeps the expandable mechanism 23016 centered relative to the frame 25100, regardless of the amount of expansion of the expandable/expansion member 23018. In some implementations, when the expandable mechanism 23016 is expanded, the proximal end 23022 slides along the rails 25102 toward the distal end 23024 and the distal end 23024 slides along the rails 25102 toward the proximal end 23022.

In some implementations, the connections between the attachment points 25110 and the attachment points 25111 (i.e., the connections between the expandable frame member 23002 and the ring 25104) and the connections between the attachment points 25112 and the attachment points 25113 (i.e., the connections between expandable frame member 23002 and the expandable/expansion member 23018) keep the midpoint 23034 of the expandable/expansion member 23018 aligned with the ring 25104 as the expandable mechanism is moved between the contracted and expanded positions.

FIGS. 119-122 illustrate an example of a device 24000 (e.g., valve repair device, valve treatment device, implantable device, implant, etc.) having an expandable coaptation element 23000. The expandable coaptation element 23000 can be used in a variety of different devices, including but not limited to, any of the treatment and/or repair devices disclosed in the present application. For example, any expandable coaptation element disclosed herein can be used in the device that is schematically illustrated in FIGS. 8-14.

The device 24000 is one of the many different configurations that the device that is schematically illustrated in FIGS. 8-14 with an expandable coaptation element 23000 can take. The device 24000 can include any other features for any device discussed in the present application, and the expandable coaptation element 23000 can be positioned to engage leaflets 30, 32, 34 (see FIGS. 7 and 34) or leaflets 20, 22 (see FIGS. 6 and 36) as part of any suitable device (e.g., any treatment and/or repair device disclosed in the present application).

In some implementations, the device 24000 can be deployed from a delivery sheath, and/or an implant catheter. In some implementations, the device 24000 can include an expandable mechanism 23016, an expandable frame or spacer 23002, and/or an anchor portion having two or more anchors, such as the anchor portion and anchors described herein.

In some implementations, the device 24000 can be a prosthetic spacer device, valve repair device, valve treatment device, implant, a treatment device, and/or another type of device that attaches to leaflets of a native valve.

Referring now to FIGS. 119-122, a device 24000 (e.g., a valve repair device, an implantable device, implant, valve treatment device, etc.) with an expandable mechanism 23016 and an expandable frame or spacer 23002 is shown. The device 24000 is one of many different configurations that the device 100 that is schematically illustrated in FIGS. 8-14 with an expandable mechanism 23016 and an expandable frame or spacer 23002 added can take.

In some implementations, the device 24000 includes an expandable mechanism 23016, an expandable frame or spacer 23002, a proximal or attachment portion 26006, an anchor portion 26008, and/or a distal portion 26010.

In some implementations, the expandable mechanism 23016 and/or expandable frame or spacer 23002 are configured for adjustable implantation between leaflets of a native valve.

In some implementations, the anchor portion 26008 includes a plurality of anchors 26014. The anchors can be configured in a variety of ways. In some implementations, the anchors 26014 include outer paddles 26016, inner paddles 26018, paddle extension members or paddle frames 26020, and clasps 26022.

In some implementations, the attachment portion 26006 includes a first or proximal collar 26030 (or other attachment element) for engaging with a capture mechanism or coupler of a delivery system.

A delivery system for the implant 24000 can be the same as or similar to the delivery system 102 described above and can comprise one or more of a catheter, a sheath, a guide catheter/sheath, a delivery catheter/sheath, a steerable catheter, an implant catheter, a tube, a channel, a pathway, combinations of these, etc.

In some implementations, portions or components of the expandable mechanism 23016, the expandable frame or spacer 23002 and/or the outer and inner paddles 26016, 26018 are formed from a flexible material that can be a metal fabric, such as mesh, woven, braided, or formed in any other suitable way or a laser cut or otherwise cut flexible material. The material can be cloth, shape-memory alloy wire—such as Nitinol—to provide shape-setting capability, or any other flexible material suitable for implantation in the human body.

An actuation element (e.g., actuation shaft, actuation rod, actuation tube, actuation wire, actuation line, etc.) can extend from a delivery system (not shown) to engage and enable actuation of the device 24000 (see actuation element 112 in FIGS. 8-14). In some implementations, the actuation element extends through the proximal collar 26030, the expandable mechanism 23016 and the expandable frame or spacer 23002 to engage a cap 26040 of the distal portion 26010. The actuation element can be configured to removably engage the cap 26040 with a threaded connection, or the like, so that the actuation element can be disengaged and removed from the implant 24000 after implantation.

The frame 25100, that the expandable mechanism 23016 is mounted to, extends from the proximal collar 26030 (or other attachment) to the cap 26040. The size and/or shape of the expandable frame or spacer 23002 can be selected and/or be adjusted to minimize the number of implants that a single patient will require (preferably one), while at the same time maintaining low transvalvular gradients.

In some implementations, the outer paddles 26016 are jointably attached to the cap 26040 of the distal portion 26010 by connection portions 26080 and to the inner paddles 26018 by transition portions 26082. In some implementations, the paddle frames 26020 are attached to the cap 26040 at the distal portion 26010 and extend to the transition portions 26082 between the inner and outer paddles 26018, 26016. In some implementations, the paddle frames 26020 are formed of a material that is more rigid and stiff than the material forming the paddles 26018, 26016 so that the paddle frames 26020 provide support for the paddles 26018, 26016.

The paddle frames 26020 can provide additional pinching force between the inner paddles 26018 and the expandable frame or spacer 23002. In some implementations, the connections between the paddle frames 26020, the outer and inner paddles 26016, 26018, the cap 26040, and the expandable frame or spacer 23002 can constrain each of these parts to the movements and positions of the treatment and/or repair devices described herein.

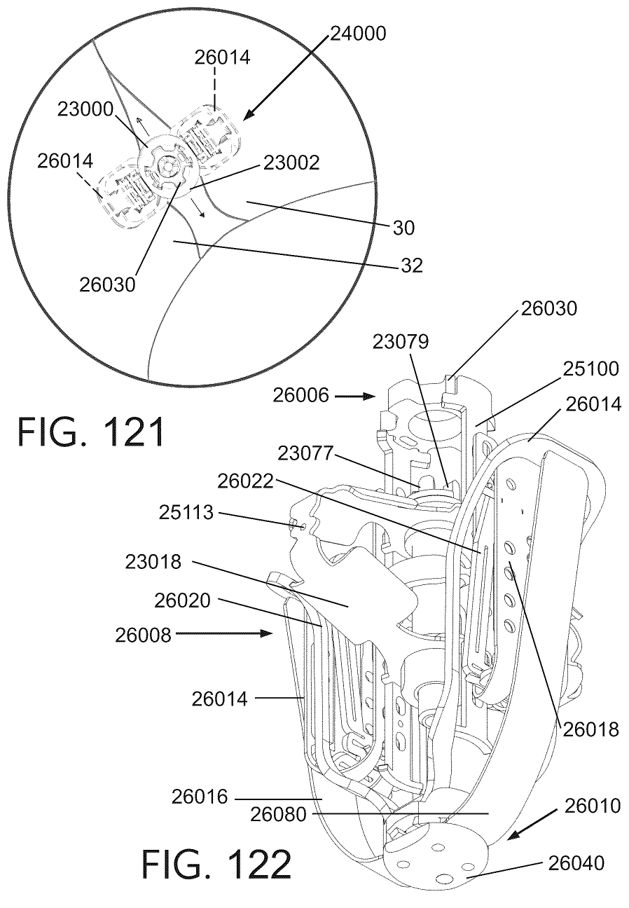

Referring now to FIG. 121, the expandable frame or spacer 23002 is positioned between the leaflets 30, 32 as shown in the top plan view. The anchors 26014 are shown in dashed lines in FIG. 121, since, with the exception of the clasps, the installed anchors 26014 are disposed on the ventricular side of the native leaflets. FIG. 120 shows the device 24000 with the expandable frame or spacer 23002 in an expanded configuration.

FIGS. 119 and 121 show the device 24000 with the expandable frame or spacer 23002 in an unexpanded, collapsed, or contracted configuration. In some implementations, adjusting the expandable mechanism 23016, for example by rotating the engagement surfaces 23077 of a head 23079, expands or contracts the expandable frame or spacer 23002 to adjust flow through the native valve (reduce regurgitation through the native valve by increasing the size of the shell or increasing the flow through the native valve by reducing the size of the shell).

Referring to FIG. 122, the strips 23028 of the expandable mechanism 23016 are spread apart to move the expandable frame or spacer 23002. As such, the expandable frame or spacer 23002 has a wide configuration and takes up a larger space. Referring to FIG. 119, the strips 23028 of the mechanism 23016 are close together to move the expandable frame or spacer 23002 to a contracted configuration. As such, the expandable frame or spacer 23002 has a narrow configuration (or unexpanded configuration) and takes up a smaller space.

Below are some non-limiting examples of some of the concepts covered herein:

Example 1. A coaptation element (e.g., an expandable coaptation element, etc.) for inhibiting regurgitation between native heart valve leaflets comprising: (i) an expandable mechanism configured to move between an expanded configuration and a collapsed configuration; and/or (ii) two or more shell components attached to the expandable mechanism.

Example 2. The coaptation element of example 1 wherein a pair of the two or more shell components nest together when the expandable mechanism is in the collapsed configuration.

Example 3. The coaptation element of any one of examples 1-2 wherein the expandable mechanism comprises a plurality of struts.

Example 4. The coaptation element of any one of examples 1-3 wherein the expandable mechanism is configured to expand in a single direction.

Example 5. The coaptation element of any one of examples 1˜4 wherein the expandable mechanism is configured to expand in two opposed directions.

Example 6. A device comprising: (A) an anchor portion configured to attach to leaflets of a native heart valve; and/or (B) a coaptation element (e.g., an expandable coaptation element, etc.) attached to the anchor portion, wherein the coaptation element comprises: (i) an expandable mechanism configured to move between an expanded configuration and a collapsed configuration; and/or (ii) two or more shell components attached to the expandable mechanism.

Example 7. The device of example 6 wherein a pair of the two or more shell components shell components nest together when the expandable mechanism is in the collapsed configuration.

Example 8. The device of any one of examples 6-7 wherein the expandable mechanism comprises a plurality of struts.

Example 9. The device of any one of examples 6-8 wherein the expandable mechanism is configured to expand in a single direction.

Example 10. The device of any one of examples 6-9 wherein the expandable mechanism is configured to expand in two opposed directions.

Example 11. A system comprising: (A) a delivery system comprising a catheter and a control handle; and/or (B) a device coupled to the delivery system, the device comprising: (i) an anchor portion configured to attach to leaflets of a native heart valve; (ii) a coaptation element (e.g., an expandable coaptation element, etc.) attached to the anchor portion, wherein the coaptation element comprises: (1) an expandable mechanism configured to move between an expanded configuration and a collapsed configuration; and/or (2) two or more shell components attached to the expandable mechanism.

Example 12. The system of example 11 wherein a pair of the two or more shell components nest together when the expandable mechanism is in the collapsed configuration.

Example 13. The system of any one of examples 11-12 wherein the expandable mechanism comprises a plurality of struts.

Example 14. The system of any one of examples 11-13 wherein the expandable mechanism is configured to expand in a single direction.

Example 15. The system of any one of examples 11-14 wherein the expandable mechanism is configured to expand in two opposed directions.

Example 16. A coaptation element (e.g., an expandable coaptation element, etc.) for inhibiting regurgitation between native heart valve leaflets comprising: (i) an expandable mechanism configured to move between an expanded configuration and a collapsed configuration; and/or (ii) an expandable sleeve disposed around the expandable mechanism.

Example 17. The coaptation element of example 16 wherein the expandable sleeve includes overlapping end portions.

Example 18. The coaptation element of any one of examples 16-17 wherein the expandable mechanism comprises a plurality of struts.

Example 19. The coaptation element of any one of examples 16-18 wherein the expandable mechanism is configured to expand in a single direction.

20. The coaptation element of any one of examples 16-19 wherein the expandable mechanism is configured to expand in two opposed directions.

Example 21. A device comprising: (A) an anchor portion configured to attach to leaflets of a native heart valve; and/or (B) a coaptation element (e.g., an expandable coaptation element, etc.) attached to the anchor portion, wherein the coaptation element comprises: (i) an expandable mechanism configured to move between an expanded configuration and a collapsed configuration; and/or an expandable sleeve disposed around the expandable mechanism.

Example 22. The device of example 21 wherein the expandable sleeve includes overlapping end portions.

Example 23. The device of any one of examples 21-22 wherein the expandable mechanism comprises a plurality of struts.

Example 24. The device of any one of examples 21-23 wherein the expandable mechanism is configured to expand in a single direction.

Example 25. The device of any one of examples 21-24 wherein the expandable mechanism is configured to expand in two opposed directions.

Example 26. A treatment and/or repair system comprising: (A) a delivery system comprising a catheter and a control handle; and (B) a valve repair device coupled to the delivery system, the valve repair device comprising: (i) an anchor portion configured to attach to leaflets of a native heart valve; and/or (ii) a coaptation element (e.g., an expandable coaptation element, etc.) attached to the anchor portion, wherein the coaptation element comprises: (1) an expandable mechanism configured to move between an expanded configuration and a collapsed configuration; and/or (2) an expandable sleeve disposed around the expandable mechanism.

Example 27. The treatment and/or repair system of example 26 wherein the expandable sleeve includes overlapping end portions.

Example 28. The treatment and/or repair system of any one of examples 26-27 wherein the expandable mechanism comprises a plurality of struts.

Example 29. The treatment and/or repair system of any one of examples 26-28 wherein the expandable mechanism is configured to expand in a single direction.

Example 30. The treatment and/or repair system of any one of examples 26-29 wherein the expandable mechanism is configured to expand in two opposed directions.

Example 31. A coaptation element (e.g., an expandable coaptation element, etc.) for inhibiting regurgitation between native heart valve leaflets comprising: (i) one or more shape changing components; and/or wherein application of a tensile force to the one or more shape changing components changes the one or more shape changing components from a flat configuration to a curved configuration.

Example 32. The coaptation element of example 31 further comprising a compressible fill material disposed between a pair of the one or more shape changing components.

Example 33. The coaptation element of any one of examples 31-32 wherein the one or more shape changing components have a kirigami configuration.

Example 34. The coaptation element of any one of examples 31-33 wherein a pair of the one or more shape changing components are parallel and spaced apart in the flat configuration.

Example 35. The coaptation element of example 34 wherein the pair of the one or more shape changing components curve toward one another in the curved configuration.

Example 36. A device comprising: (A) an anchor portion configured to attach to leaflets of a native heart valve; and/or (B) a coaptation element (e.g., an expandable coaptation element, etc.) attached to the anchor portion, wherein the coaptation element comprises: (i) one or more shape changing components; and/or (ii) wherein application of a tensile force to the one or more shape changing components changes the one or more shape changing components from a flat configuration to a curved configuration.

Example 37. The device of example 36 further comprising a compressible fill material disposed between a pair of the one or more shape changing components.

Example 38. The device of any one of examples 36-37 wherein the one or more shape changing components have a kirigami configuration.

Example 39. The device of any one of examples 36-38 wherein a pair of the one or more shape changing components are parallel and spaced apart in the flat configuration.

Example 40. The device of example 39 wherein the pair of the one or more shape changing components curve toward one another in the curved configuration.

Example 41. A system comprising: (A) a delivery system comprising a catheter and a control handle; (B) a device coupled to the delivery system, the device comprising: (i) an anchor portion configured to attach to leaflets of a native heart valve; and/or (ii) a coaptation element (e.g., an expandable coaptation element, etc.) attached to the anchor portion, wherein the coaptation element comprises: (1) one or more shape changing components; and/or (2) wherein application of a tensile force to the one or more shape changing components changes the one or more shape changing components from a flat configuration to a curved configuration.

Example 42. The system of example 41 further comprising a compressible fill material disposed between a pair of the one or more shape changing components.

Example 43. The system of any one of examples 41-42 wherein the one or more shape changing components have a kirigami configuration.

Example 44. The system of any one of examples 41-43 wherein a pair of the one or more shape changing components are parallel and spaced apart in the flat configuration.

Example 45. The system of example 44 wherein the pair of the one or more shape changing components curve toward one another in the curved configuration.

Example 46. A coaptation element (e.g., an expandable coaptation element, etc.) for inhibiting regurgitation between native heart valve leaflets comprising: (A) a lattice of cells comprising: (i) a first controllable cell configured such that a size of the first controllable cell can be increased and decreased; (ii) a second controllable cell configured such that a size of the second controllable cell can be increased and decreased; and/or (ii) wherein the size of the first controllable cell is configured to be controlled independently of the size of the second controllable cell.

Example 47. A device comprising: (A) an anchor portion configured to attach to leaflets of a native heart valve; and/or (B) a lattice of cells connected to the anchor portion, the lattice of cells comprising: (i) a first controllable cell configured such that a size of the first controllable cell can be increased and decreased; (ii) a second controllable cell configured such that a size of the second controllable cell can be increased and decreased; and/or (iii) wherein the size of the first controllable cell is configured to be controlled independently of the size of the second controllable cell.

Example 48. A system comprising: (i) a first control element; (ii) a second control element; (iii) a lattice of cells comprising: (1) a first controllable cell coupled to the first control element, wherein movement of the first control element changes a size of the first controllable cell; and/or (2) a second controllable cell coupled to the second control element, wherein movement of the second control element changes a size of the second controllable cell.

Example 49. The system of example 48 wherein the first control element is configured to move in a direction of a height of the first controllable cell.

Example 50. The system of example 49 wherein the second control element is configured to apply force in a direction of a width of the second controllable cell.

Example 51. A coaptation element (e.g., an expandable coaptation element, etc.) for inhibiting regurgitation between native heart valve leaflets comprising: (i) a receiver; a shape changing element having a first end disposed in the receiver and a second end disposed outside the receiver; (ii) a shaft disposed in the receiver and connected to the first end of the shape changing element; and/or (iii) wherein pushing the shaft in the receiver pushes a portion of the shape changing element out of the receiver to increase a size of the shape changing element.

Example 52. The coaptation element of example 51 wherein the shape changing element comprises a plurality of wires.

Example 53. The coaptation element of any one of examples 51-52 wherein the shape changing element comprises a braided or mesh material.

Example 54. The coaptation element of any one of examples 51-53 wherein the shape changing element has a teardrop shape in an expanded condition.

Example 55. The coaptation element of example 54 wherein the shape changing element has a substantially cylindrical configuration in a retracted condition.

Example 56. A device comprising: (A) an anchor portion configured to attach to leaflets of a native heart valve; and/or (B) a coaptation element (e.g., an expandable coaptation element, etc.) attached to the anchor portion, wherein the coaptation element comprises: (i) a receiver; (ii) a shape changing element having a first end disposed in the receiver and a second end disposed outside the receiver; (iii) a shaft disposed in the receiver and connected to the first end of the shape changing element; and/or (iv) wherein pushing the shaft in the receiver pushes a portion of the shape changing element out of the receiver to increase a size of the shape changing element.

Example 57. The device of example 56 wherein the shape changing element comprises a plurality of wires.

Example 58. The device of any one of examples 56-57 wherein the shape changing element comprises a braided or mesh material.

Example 59. The device of any one of examples 56-58 wherein the shape changing element has a teardrop shape in an expanded condition.

Example 60. The device of example 59 wherein the shape changing element has a substantially cylindrical configuration in a retracted condition.

Example 61. A treatment and/or repair system comprising: (A) a delivery system comprising a catheter and a control handle; and/or (B) a valve repair device coupled to the delivery system, the valve repair device comprising: (i) an anchor portion configured to attach to leaflets of a native heart valve; and/or (ii) a coaptation element (e.g., an expandable coaptation element, etc.) attached to the anchor portion, wherein the coaptation element comprises: (1) a receiver; (2) a shape changing element having a first end disposed in the receiver and a second end disposed outside the receiver; and/or (3) a shaft disposed in the receiver and connected to the first end of the shape changing element; and/or wherein pushing the shaft in the receiver with the delivery system pushes a portion of the shape changing element out of the receiver to increase a size of the shape changing element.

Example 62. The treatment and/or repair system of example 61 wherein the shape changing element comprises a plurality of wires.

Example 63. The treatment and/or repair system of any one of examples 61-62 wherein the shape changing element comprises a braided or mesh material.

Example 64. The treatment and/or repair system of any one of examples 61-63 wherein the shape changing element has a teardrop shape in an expanded condition.

Example 65. The treatment and/or repair system of example 64 wherein the shape changing element has a substantially cylindrical configuration in a retracted condition.

Example 66. A valve repair device comprising: (i) an anchor portion configured to attach to leaflets of a native heart valve; (ii) an extension attached to the anchor portion; and/or (iii) wherein the extension is configured to impede regurgitant flow through the native heart valve.

Example 67. The valve repair device of example 66 wherein the extension is expandable.

Example 68. A system comprising: (i) a catheter; (ii) a device coupled to the catheter; and/or (iii) an extension configured to slide over the catheter and attach to the device.

Example 69. The system of example 68 wherein the extension is expandable.

Example 70. The system of any one of examples 68-69 wherein the device comprises a coaptation element configured to engage leaflets of a native heart valve.

Example 71. A coaptation element (e.g., an expandable coaptation element, etc.) for inhibiting regurgitation between native heart valve leaflets comprising: (A) an expandable frame member; and/or (B) an expandable mechanism mounted within the expandable frame member, the expandable mechanism comprising: (i) an expandable/expansion member connected to the expandable frame member; and/or (ii) an actuation mechanism mounted inside the expandable/expansion member, the actuation mechanism configured to move the expandable/expansion member and the expandable frame member between an expanded configuration and a collapsed configuration.

Example 72. The coaptation element of example 71, wherein the expandable/expansion member includes a distal end portion, a proximal end portion opposite the distal end portion, and an intermediate portion between the distal end portion and the proximal end portion, wherein the intermediate portion expands when the expandable frame member moves from the collapsed configuration to the expanded configuration.

Example 73. The coaptation element of example 72, wherein the intermediate portion comprises a plurality of strips the extend longitudinally and are spaced apart, the plurality of strips being configured to bend when the intermediate portion expands.

Example 74. The coaptation element of example 73, wherein the intermediate portion comprises between 4 and 8 equally spaced apart strips.

Example 75. The coaptation element of example 73 or 74, wherein each of the plurality of strips is attached to the expandable frame member.

Example 76. The coaptation element of any of examples 72-75, wherein the actuation mechanism comprises a distal member axially fixed relative to the distal end portion of the expandable/expansion member and a proximal member is axially fixed relative to the proximal end portion of the expandable/expansion member.

Example 77. The coaptation element of example 76, wherein the distal member includes a pair of projections received within a pair of openings in the distal end portion of the expandable/expansion member.

Example 78. The coaptation element of example 76, wherein the distal member is axially fixed to the distal end portion of the expandable/expansion member by a stop positioned within a passage of the expandable/expansion member.

Example 79. The coaptation element of example 76, wherein a portion of the proximal member is axially fixed relative to the distal end portion between a stop positioned within a passage of the expandable/expansion member and an end cap received within the passage at the proximal end portion of the expandable/expansion member.

Example 80. The coaptation element of any one of examples 76-79, wherein relative movement of the distal member and the proximal member toward each other causes expansion of the intermediate portion.

Example 81. The coaptation element of example 80, wherein the distal member and the proximal member are threadably coupled and relative rotation between the distal member and the proximal member causes relative movement of the distal member and the proximal member toward each other.

Example 82. The coaptation element of example 80, wherein a distal end of the proximal member is received within a passage of the distal member, and wherein the distal end of the proximal member includes male threads that are threadably coupled to female threads in the passage of the distal member.

Example 83. The coaptation element of example 80, wherein a proximal end of the distal member is received within a passage of the proximal member, and wherein the distal member is axially moveable within the passage of the proximal member.

Example 84. The coaptation element of example 83, wherein the distal member includes one or more locks configured to lock an axial position of the distal member relative to the proximal member.

Example 85. The coaptation element of example 84, wherein each of the one or more locks engage a corresponding slot in the proximal member to lock the axial position of the distal member relative to the proximal member.

Example 86. The coaptation element of example 83, wherein the proximal end of the distal member includes a coupling portion configured to be engaged by an actuation element extending through the passage in the proximal member to move the distal member is axially moveable within the passage.

Example 87. The coaptation element of any of examples 71-86, wherein the expandable frame member has a height that remains the same between the collapsed configuration and the expanded configuration.

Example 88. The coaptation element of any of examples 71-86, wherein the expandable frame member comprises a plurality of fixed height posts interconnected by a plurality of struts.

Example 89. The coaptation element of any of example 88, wherein the plurality of struts are arranged in a plurality of diamond shape patterns.

Example 90. The coaptation element of any of example 88, wherein the expandable/expansion member is connected to one or more fixed height posts of the expandable frame member.

Example 91. The coaptation element of any of examples 71-90, further comprising a stretchable cover that covers at least a portion of the expandable frame member.

Example 92. The coaptation element of example 91, wherein the stretchable cover is connected to the expandable frame member.

Example 93. The coaptation element of example 91 or 92, wherein the stretchable cover comprises a plurality of weave portions spaced apart and connected by a plurality of stretchable portions.

Example 94. The coaptation element of example 93, wherein the expandable frame member comprises a plurality of fixed height posts interconnected by a plurality of struts and wherein one or more of the plurality of weave portions are connected to a corresponding one or more of the plurality of fixed height posts.

Example 95. The coaptation element of example 94, wherein the expandable frame member comprises six posts and the stretchable cover comprises six weave portions, each of the plurality of weave portions being connected to a corresponding one of the plurality of fixed height posts.

Example 96. The coaptation element of example 93, wherein each weave portion of the plurality of weave portions is connected to a corresponding stretchable portion by a Leno stitch.

Example 97. A system, comprising: (A) a delivery system comprising a catheter and a control handle; and/or (B) a device coupled to the delivery system, the device comprising: (i) an anchor portion configured to attach to leaflets of a native heart valve; and/or (ii) a coaptation element (e.g., an expandable coaptation element, etc.) attached to the anchor portion, wherein the coaptation element comprises: (1) an expandable frame member; and/or (2) an expandable mechanism mounted within the expandable frame member, the expandable mechanism comprising: (a) an expandable/expansion member connected to the expandable frame member; and/or (b) an actuation mechanism mounted inside the expandable/expansion member, the actuation mechanism configured to move the expandable/expansion member and the expandable frame member between an expanded configuration and a collapsed configuration.

Example 98. The system of example 97, wherein the expandable/expansion member includes a distal end portion, a proximal end portion opposite the distal end portion, and an intermediate portion between the distal end portion and the proximal end portion, wherein the intermediate portion expands when the expandable frame member moves from the collapsed configuration to the expanded configuration.

Example 99. The system of example 98, wherein the intermediate portion comprises a plurality of strips the extend longitudinally and are spaced apart, the plurality of strips being configured to bend when the intermediate portion expands.

Example 100. The system of example 99, wherein the intermediate portion comprises between 4 and 8 equally spaced apart strips.

Example 101. The system of example 99 or 100, wherein each of the plurality of strips is attached to the expandable frame member.

Example 102. The system of any of examples 98-101, wherein the actuation mechanism comprises a distal member axially fixed relative to the distal end portion of the expandable/expansion member and a proximal member is axially fixed relative to the proximal end portion of the expandable/expansion member.

Example 103. The system of example 102, wherein the distal member includes a pair of projections received within a pair of openings in the distal end portion of the expandable/expansion member.

Example 104. The system of example 102, wherein the distal member is axially fixed to the distal end portion of the expandable/expansion member by a stop positioned within a passage of the expandable/expansion member.

Example 105. The system of example 102, wherein a portion of the proximal member is axially fixed relative to the distal end portion between a stop positioned within a passage of the expandable/expansion member and an end cap received within the passage at the proximal end portion of the expandable/expansion member.

Example 106. The system of any one of examples 102-105, wherein relative movement of the distal member and the proximal member toward each other causes expansion of the intermediate portion.

Example 107. The system of example 106, wherein the distal member and the proximal member are threadably coupled and relative rotation between the distal member and the proximal member causes relative movement of the distal member and the proximal member toward each other.

Example 108. The system of example 106, wherein a distal end of the proximal member is received within a passage of the distal member, and wherein the distal end of the proximal member includes male threads that are threadably coupled to female threads in the passage.

Example 109. The system of example 106, wherein a proximal end of the distal member is received within a passage of the proximal member, and wherein the distal member is axially moveable within the passage of the proximal member.

Example 110. The system of example 109, wherein the distal member includes one or more locks configured to lock an axial position of the distal member relative to the proximal member.

Example 111. The system of example 110, wherein each of the one or more locks engage a corresponding slot in the proximal member to lock the axial position of the distal member relative to the proximal member.

Example 112. The system of example 109, wherein the proximal end of the distal member includes a coupling portion configured to be engaged by an actuation element extending through the passage in the proximal member to move the distal member is axially moveable within the passage.

Example 113. The system of any of examples 97-112, wherein the expandable frame member has a height that remains the same between the collapsed configuration and the expanded configuration.

Example 114. The system of any of examples 97-112, wherein the expandable frame member comprises a plurality of fixed height posts interconnected by a plurality of struts.

Example 115. The system of any of example 114, wherein the plurality of struts are arranged in a plurality of diamond shape patterns.

Example 116. The system of any of example 114, wherein the expandable/expansion member is connected to one or more fixed height posts of the expandable frame member.

Example 117. The system of any of examples 97-116, further comprising a stretchable cover that covers at least a portion of the expandable frame member.

Example 118. The system of example 117, wherein the cover is connected to the expandable frame member.

Example 119. The system of example 117 or 118, wherein the cover comprises a plurality of spaced apart weave portions connected by a plurality of stretchable portions.

Example 120. The system of any one of examples 117-119, wherein the stretchable cover is treated to decrease the permeability of the cover.

Example 121. The system of example 120, wherein the stretchable cover is coated with a polymer to decrease the permeability of the cover.

Example 122. The system of example 120, wherein one or more polymer strands made from one or more of TPU, silicone, polyolefin and elastic yarn are woven into the stretchable cover to decrease the permeability of the cover.

Example 123. A method of inhibiting regurgitation between native heart valve leaflets, comprising: (i) positioning a device between the valve leaflets; (ii) attaching an anchor portion of the device to the valve leaflets; (iii) expanding a coaptation element attached to the anchor portion; and/or (iv) wherein expanding the coaptation element further comprises expanding an expandable/expansion member positioned within a frame member to move the frame member from a collapsed position to an expanded position.

Example 124. The method of example 123, wherein expanding the expandable/expansion member further comprises bending a plurality of strips on the expandable/expansion member.

Example 125. The method of any one of examples 123 or 124, wherein the frame member has a height that remains the same in both the collapsed position and the expanded position.

Example 126. The method of any one of examples 123-125, wherein the expanding the expandable/expansion member further comprises axially moving one of a distal member and a proximal member relative to the other of the distal member and a proximal member within a passage of the expandable/expansion member.

Example 127. The method of example 126, wherein axially moving one of a distal member and a proximal member relative to the other of the distal member and a proximal member further comprises rotating the proximal member relative to the distal member.

Example 128. The method of example 126, wherein axially moving one of a distal member and a proximal member relative to the other of the distal member and a proximal member further comprises threading the proximal member into a passage of the distal member.

Example 129. The method of example 126, wherein axially moving one of a distal member and a proximal member relative to the other of the distal member and a proximal member further comprises axially moving the distal member within a passage of the proximal member.

Example 130. The method of example 129, wherein further comprising locking the position of the distal member within the passage of the proximal member.

Example 131. The method of example 125, further comprising inhibiting axial movement of the proximal member while allowing rotation movement.

Example 132. A coaptation element (e.g., an expandable coaptation element, etc.) for inhibiting regurgitation between native heart valve leaflets comprising: (i) an expandable mechanism configured to move between an expanded configuration and a collapsed configuration; and/or (ii) an expandable frame attached around the expandable mechanism; wherein the expandable mechanism is configured to expand in only two opposite directions.

Example 133. The coaptation element of example 132 wherein the expandable frame has a circular cross-section when the expandable mechanism is in the collapsed configuration and the expandable frame has an oval cross-section when the expandable mechanism is in the expanded configuration.

Example 134. The coaptation element of any one of examples 132-133 wherein the expandable mechanism comprises a plurality of struts.

The techniques, methods, operations, steps, etc. described or suggested herein (including in the examples above) or in the references incorporated herein can be performed on a living subject (e.g., human, other animal, etc.) or on a simulation, such as a cadaver, cadaver heart, simulator, imaginary person, etc.). When performed on a simulation, the body parts, e.g., heart, tissue, valve, etc., can be assumed to be simulated or can optionally be referred to as “simulated” (e.g., simulated heart, simulated tissue, simulated valve, etc.) and can optionally comprise computerized and/or physical representations of body parts, tissue, etc. The term “simulation” covers use on a cadaver, computer simulator, imaginary person (e.g., if they are just demonstrating in the air on an imaginary heart), etc.

Any of the various systems, assemblies, devices, components, apparatuses, etc. in this disclosure (including those in the examples above) can be sterilized (e.g., with heat, radiation, ethylene oxide, hydrogen peroxide, etc.) to ensure they are safe for use with patients, and the methods herein can comprise (or additional methods comprise or consist of) sterilization of the associated system, device, component, apparatus, etc. (e.g., with heat, radiation, ethylene oxide, hydrogen peroxide, etc.

While various inventive aspects, concepts and features of the disclosures can be described and illustrated herein as embodied in combination in the examples herein, these various aspects, concepts, and features can be used in many alternative examples, either individually or in various combinations and sub-combinations thereof. Unless expressly excluded herein all such combinations and sub-combinations are intended to be within the scope of the present application. Still further, while various alternative examples as to the various aspects, concepts, and features of the disclosures—such as alternative materials, structures, configurations, methods, devices, and components, alternatives as to form, fit, and function, and so on—may be described herein, such descriptions are not intended to be a complete or exhaustive list of available alternative examples, whether presently known or later developed. Those skilled in the art can readily adopt one or more of the inventive aspects, concepts, or features into additional examples and uses within the scope of the present application even if such examples are not expressly disclosed herein.

Additionally, even though some features, concepts, or aspects of the disclosures may be described herein as being a preferred arrangement or method, such description is not intended to suggest that such feature is required or necessary unless expressly so stated. Still further, example or representative values and ranges may be included to assist in understanding the present application, however, such values and ranges are not to be construed in a limiting sense and are intended to be critical values or ranges only if so expressly stated.

Moreover, while various aspects, features and concepts may be expressly identified herein as being inventive or forming part of a disclosure, such identification is not intended to be exclusive, but rather there may be inventive aspects, concepts, and features that are fully described herein without being expressly identified as such or as part of a specific disclosure, the disclosures instead being set forth in the appended claims. Descriptions of example methods or processes are not limited to inclusion of all steps as being required in all cases, nor is the order that the steps are presented to be construed as required or necessary unless expressly so stated. The words used in the claims have their full ordinary meanings and are not limited in any way by the description of the examples in the specification.

What is claimed is:

1. An expandable coaptation element for inhibiting regurgitation between native heart valve leaflets comprising:
an expandable mechanism configured to move between an expanded configuration and a collapsed configuration;
an expandable frame attached around the expandable mechanism; and
wherein the expandable mechanism is configured to expand in only two opposite directions.

2. The expandable coaptation element of claim 1 wherein the expandable frame has a circular cross-section when the expandable mechanism is in the collapsed configuration and the expandable frame has an oval cross-section when the expandable mechanism is in the expanded configuration.

3. The expandable coaptation element of claim 1 wherein the expandable frame comprises two or more shell components attached to the expandable mechanism.

4. The expandable coaptation element of claim 3 wherein a pair of the two or more shell components nest together when the expandable mechanism is in the collapsed configuration.

5. The expandable coaptation element of claim 1 wherein the expandable frame comprises an expandable sleeve disposed around the expandable mechanism.

6. The expandable coaptation element of claim 5 wherein the expandable sleeve includes overlapping end portions.

7. The expandable coaptation element of claim 1 wherein the expandable mechanism comprises a plurality of struts.

8. The expandable coaptation element of claim 1 further comprising an actuation mechanism mounted inside the expandable mechanism, the actuation mechanism configured to move the expandable mechanism and the expandable frame between the expanded configuration and the collapsed configuration.

9. The expandable coaptation element of claim 8, wherein the expandable mechanism includes a distal end portion, a proximal end portion opposite the distal end portion, and an intermediate portion between the distal end portion and the proximal end portion, wherein the intermediate portion expands when the expandable frame moves from the collapsed configuration to the expanded configuration.

10. The expandable coaptation element of claim 9, wherein the intermediate portion comprises a plurality of strips the extend longitudinally and are spaced apart, the plurality of strips being configured to bend when the intermediate portion expands.

11. The expandable coaptation element of claim 1, further comprising a stretchable cover that covers at least a portion of the expandable frame.

12. The expandable coaptation element of claim 11, wherein the stretchable cover comprises a plurality of weave portions spaced apart and connected by a plurality of stretchable portions.

13. The expandable coaptation element of claim 12, wherein the expandable frame comprises a plurality of fixed height posts interconnected by a plurality of struts and wherein one or more of the plurality of weave portions are connected to a corresponding one or more of the plurality of fixed height posts.

14. A coaptation element for inhibiting regurgitation between native heart valve leaflets comprising:
one or more shape changing components; and
wherein application of a tensile force to the one or more shape changing components changes the one or more shape changing components from a flat configuration to a curved configuration.

15. The coaptation element of claim 14 further comprising a compressible fill material disposed between a pair of the one or more shape changing components.

16. The coaptation element of claim 14 wherein the one or more shape changing components have a kirigami configuration.

17. The coaptation element of claim 14 wherein the one or more shape changing components comprise:
a lattice of cells comprising:
a first controllable cell configured such that a size of the first controllable cell can be increased and decreased;
a second controllable cell configured such that a size of the second controllable cell can be increased and decreased; and
wherein the size of the first controllable cell is configured to be controlled independently of the size of the second controllable cell.

18. The coaptation element of claim 14 wherein the shape changing component has a first end disposed in a receiver and a second end disposed outside the receiver, wherein a shaft is disposed in the receiver and connected to the first end of the shape changing component, and wherein pushing the shaft in the receiver pushes a portion of the shape changing component out of the receiver to increase a size of the shape changing component.

19. The coaptation element of claim 14 wherein the shape changing component comprises a plurality of wires.

20. A treatment and/or repair device comprising:
an anchor portion configured to attach to leaflets of a native heart valve;
an extension attached to the anchor portion; and
wherein the extension is configured to impede regurgitant flow through the native heart valve.
